SEQUENCE LISTING (211 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 3 - DNA, 108 bp, Artificial Sequence
  Oligo DNA encoding randomized amino acid sequence
  misc_feature (34)..(35): n is a, c, g, or t
  misc_feature (37)..(38): n is a, c, g, or t
  misc_feature (40)..(41): n is a, c, g, or t
  misc_feature (43)..(44): n is a, c, g, or t
  misc_feature (46)..(47): n is a, c, g, or t
  misc_feature (49)..(50): n is a, c, g, or t
  misc_feature (52)..(53): n is a, c, g, or t
  misc_feature (55)..(56): n is a, c, g, or t
  misc_feature (58)..(59): n is a, c, g, or t
  misc_feature (61)..(62): n is a, c, g, or t
  misc_feature (64)..(65): n is a, c, g, or t
  misc_feature (67)..(68): n is a, c, g, or t
gactataaagatgacgatgacaaaggcgggtgcnnsnnsnnsnnsnnsnnsnnsnnsnnsnnsnnsnnstgtggaggcgagcagaagctgatctctgaggaggatctg

SEQ ID NO: 4 - protein, 36 aa, Artificial Sequence
  cyclic peptide with randomized amino acid sequence
  misc_feature (12)..(23): Xaa can be any naturally occurring amino acid
DYKDDDDKGGCXXXXXXXXXXXXCGGEQKLISEEDL

SEQ ID NO: 5 - DNA, 112 bp, Artificial Sequence
  5′UTR Oligo DNA
gaaattaatacgactcactatagggagaccacaacggtttccctctagaaataattttgtttaactttaagaaggagatataccaatggactataaagatgacgatgacaaa

SEQ ID NO: 6 - DNA, 60 bp, Artificial Sequence
  Primer Myc-g3p
gagcagaagctgatctctgaggaggatctgaagcttgaatatcaaggccaatcgtctgac

SEQ ID NO: 7 - DNA, 108 bp, Artificial Sequence
  Primer g3p-SecMstop
ctcgagttattcattaggtgaggcgttgagggccagcacggatgccttgcgcctggcttatccagacgggcgtgctgaattttgcgccggaaacgtcaccaatgaaac

SEQ ID NO: 8 - DNA, 48 bp, Artificial Sequence
  5′ primer
gaaattaatacgactcactatagggagaccacaacggtttccctctag

SEQ ID NO: 9 - DNA, 32 bp, Artificial Sequence
  Primer SecMstop
ggattagttattcattaggtgaggcgttgagg

SEQ ID NO: 10 - DNA, 22 bp, Artificial Sequence
  RT reverse primer Myc R
cagatcctcctcagagatcagc

SEQ ID NO: 11 - DNA, 29 bp, Artificial Sequence
  Primer FLAG F
atggactataaagatgacgatgacaaagg

SEQ ID NO: 12 - DNA, 35 bp, Artificial Sequence
  Primer Eco1-(-M)-FLAG_F
ccgaattcgactataaagatgacgatgacaaaggc

SEQ ID NO: 13 - DNA, 33 bp, Artificial Sequence
  Primer Myc-Hind3_R
aaaaaaaaaagcttcagatcctcctcagagatc

SEQ ID NO: 14 - DNA, 28 bp, Artificial Sequence
  Primer pET-MALseqF
ccagaaaggtgaaatcatgccgaacatc

SEQ ID NO: 15 - protein, 14 aa, Artificial Sequence
  H12SSI-Ctla4-3-02
CMHPFLLVVSHHFC

SEQ ID NO: 16 - protein, 14 aa, Artificial Sequence
  H12SSI-Ctla4-3-51
CGLGQGYWFKVWFC

SEQ ID NO: 17 - protein, 14 aa, Artificial Sequence
  H12SSI-Ctla4-3-33
CAKNFGYWYHQWFC

SEQ ID NO: 18 - protein, 14 aa, Artificial Sequence
  H12SSI-Ctla4-3-80
CGAGKGFWFKVWFC

SEQ ID NO: 19 - protein, 14 aa, Artificial Sequence
  H12SSI-Ctla4-2-23
CGFHDGFWYNVWFC

SEQ ID NO: 20 - protein, 14 aa, Artificial Sequence
  H12SSI-Ctla4-3-90
CGVNLGFWFNVWFC

SEQ ID NO: 21 - protein, 14 aa, Artificial Sequence
  H12SSI-Ctla4-3-38
CIIQGQYWWNIWHC

SEQ ID NO: 22 - protein, 16 aa, Artificial Sequence
  Clone #19
CMHPFLPIVSHHFCER

SEQ ID NO: 23 - protein, 15 aa, Artificial Sequence
  Original clone
KCMHPFLLVVSHHFC

SEQ ID NO: 24 - protein, 17 aa, Artificial Sequence
  Clone #19
KCMHPFLPIVSHHFCER

SEQ ID NO: 25 - protein, 15 aa, Artificial Sequence
  Original clone
GCMHPFLLVVSHHFC

SEQ ID NO: 26 - protein, 17 aa, Artificial Sequence
  Clone #19
GCMHPFLPIVSHHFCER

SEQ ID NO: 27 - protein, 16 aa, Artificial Sequence
  Clone #10
CLHPFLPIVSHHFCGR

SEQ ID NO: 28 - DNA, 114 bp, Artificial Sequence
  Randomized oligo DNA
  misc_feature (34)..(35): n is a, c, g, or t
  misc_feature (43)..(44): n is a, c, g, or t
  misc_feature (55)..(56): n is a, c, g, or t
  misc_feature (61)..(62): n is a, c, g, or t
  misc_feature (73)..(74): n is a, c, g, or t
  misc_feature (76)..(77): n is a, c, g, or t
gactataaagatgacgatgacaaaggcggttgcnnscatccannsctgccgatannstcannscatttctgtnnsnnsggatccgagcagaagctgatctctgaggaggatctg

SEQ ID NO: 29 - protein, 18 aa, Artificial Sequence
  Original clone
GGCMHPFLLVVSHHFCGG

SEQ ID NO: 30 - protein, 18 aa, Artificial Sequence
  C4-302mat-1-1-24
GGCMHPFLPVVSHHFCGG

SEQ ID NO: 31 - protein, 18 aa, Artificial Sequence
  C4-302mat-2-100-18
GGCMHPFLPVVSHHFCRG

SEQ ID NO: 32 - protein, 18 aa, Artificial Sequence
  C4-302mat-3-1-48
GGCMHPFLPVVSHHFCGA

SEQ ID NO: 33 - protein, 18 aa, Artificial Sequence
  C4-302mat-3-100-37
GGCMHPFLPVVSHHFCGR

SEQ ID NO: 34 - protein, 18 aa, Artificial Sequence
  C4-302mat-1-1-35
GGCMHPFLPVASHHFCGG

SEQ ID NO: 35 - protein, 18 aa, Artificial Sequence
  C4-302mat-3-1-22
GGCMHPFLPVASHHFCGR

SEQ ID NO: 36 - protein, 18 aa, Artificial Sequence
  C4-302mat-3-100-27
GGCMHPFLPVASHHFCAG

SEQ ID NO: 37 - protein, 18 aa, Artificial Sequence
  GGCLHPFLPVVSHHFCGD
GGCLHPFLPVVSHHFCGD

SEQ ID NO: 38 - protein, 18 aa, Artificial Sequence
  C4-302mat-2-1-38
GGCLHPFLPLVSHHFCGG

SEQ ID NO: 39 - protein, 18 aa, Artificial Sequence
  C4-302mat-3-100-44
GGCLHPFLPIVSHHFCGR

SEQ ID NO: 40 - protein, 18 aa, Artificial Sequence
  C4-302mat-3-1-45
GGCRHPFLPIVSRHFCRG

SEQ ID NO: 41 - protein, 18 aa, Artificial Sequence
  C4-302mat-3-100-20
GGCVHPFLPVVSHHFCRG

SEQ ID NO: 42 - protein, 18 aa, Artificial Sequence
  C4-302mat-3-100-9
GGCKHPFLPVVSHHFCGG

SEQ ID NO: 43 - protein, 18 aa, Artificial Sequence
  C4-302mat-3-1-10
GGCKHPYLPVASHHFCRG

SEQ ID NO: 44 - protein, 18 aa, Artificial Sequence
  C4-302mat-2-1-27
GGCMHPYLPVISHHFCGG

SEQ ID NO: 45 - protein, 18 aa, Artificial Sequence
  C4-302mat-3-100-12
GGCMHPYLPVISHHFCGR

SEQ ID NO: 46 - protein, 18 aa, Artificial Sequence
  C4-302mat-2-1-3
GGCMHPFLPVTSHHFCGG

SEQ ID NO: 47 - protein, 18 aa, Artificial Sequence
  C4-302mat-2-100-27
GGCMHPFLPIVSHHFCGS

SEQ ID NO: 48 - protein, 18 aa, Artificial Sequence
  C4-302mat-3-1-27
GGCMHPFLPIVSHHFCER

SEQ ID NO: 49 - protein, 18 aa, Artificial Sequence
  C4-302mat-3-1-23
GGCMHPFLPIASLHFCGR

SEQ ID NO: 50 - protein, 18 aa, Artificial Sequence
  C4-302mat-3-1-32
GGCMHPFLPILSHHFCAG

SEQ ID NO: 51 - protein, 18 aa, Artificial Sequence
  C4-302mat-3-1-31
GSCMHPFLPVASRHFCGR

SEQ ID NO: 52 - protein, 18 aa, Artificial Sequence
  C4-302mat-3-1-5
GGCMHPFLPVVSRHFCGG

SEQ ID NO: 53 - protein, 18 aa, Artificial Sequence
  C4-302mat-3-1-12
GGCMHPFLPVVSRHFCGR

SEQ ID NO: 54 - protein, 18 aa, Artificial Sequence
  C4-302mat-3-1-35
GGCMHPFLQTVSHHFCGG

SEQ ID NO: 55 - protein, 18 aa, Artificial Sequence
  C4-302mat-2-1-35
GGCMHPFLQVVSHHFCGS

SEQ ID NO: 56 - protein, 18 aa, Artificial Sequence
  C4-302mat-2-100-4
GGCMHPFLQVVSHHFCGG

SEQ ID NO: 57 - protein, 18 aa, Artificial Sequence
  C4-302mat-1-1-21
GGCIHPFLQVVSHHFCGG

SEQ ID NO: 58 - protein, 18 aa, Artificial Sequence
  C4-302mat-1-100-23
GGCVHPFLQVVSHHFCGG

SEQ ID NO: 59 - protein, 18 aa, Artificial Sequence
  C4-302mat-2-1-40
GGCMHPFLQVASHHFCGG

SEQ ID NO: 60 - protein, 18 aa, Artificial Sequence
  C4-302mat-3-1-2
GGCMHPYLQVVSHHFCGR

SEQ ID NO: 61 - protein, 18 aa, Artificial Sequence
  C4-302mat-1-1-37
GGCMHPFLRVVSHHFCGG

SEQ ID NO: 62 - protein, 18 aa, Artificial Sequence
  C4-302mat-3-1-14
GGCMHPFLRVVSHHFCGR

SEQ ID NO: 63 - protein, 18 aa, Artificial Sequence
  C4-302mat-2-1-12
GGCKHPFLRVVSHHFCRR

SEQ ID NO: 64 - protein, 18 aa, Artificial Sequence
  C4-302mat-2-1-24
GGCMHPFLRIVSQHFCRR

SEQ ID NO: 65 - protein, 18 aa, Artificial Sequence
  C4-302mat-3-1-8
GGCMHPFLKIASHHFCGG

SEQ ID NO: 66 - protein, 18 aa, Artificial Sequence
  C4-302mat-2-1-37
GGCMHPFLLVASHHFCGV

SEQ ID NO: 67 - protein, 18 aa, Artificial Sequence
  C4-302mat-2-100-33
GSCMHPFLLVASHHFCRG

SEQ ID NO: 68 - protein, 18 aa, Artificial Sequence
  C4-302mat-3-1-9
GGCMHPYLLVASHHFCER

SEQ ID NO: 69 - protein, 18 aa, Artificial Sequence
  C4-302mat-2-100-24
GGCMHPYLLVVSHHFCGR

SEQ ID NO: 70 - protein, 18 aa, Artificial Sequence
  C4-302mat-3-1-15
GGCMHPYLLVVSRHFCGR

SEQ ID NO: 71 - protein, 18 aa, Artificial Sequence
  C4-302mat-3-1-25
GDCKHPYLLTVSHHFCGR

SEQ ID NO: 72 - protein, 18 aa, Artificial Sequence
  C4-302mat-2-100-40
GGCMHPFLLVVSRHFCGR

SEQ ID NO: 73 - protein, 18 aa, Artificial Sequence
  C4-302mat-3-1-11
GGCMHPFLLTVSRHFCGG

SEQ ID NO: 74 - protein, 18 aa, Artificial Sequence
  C4-302mat-3-100-39
GGCKHPFLLIVSHHFCGR

SEQ ID NO: 75 - protein, 18 aa, Artificial Sequence
  C4m#19-5th4w-NN6-67
GGCLHPFLPIRSVHFCPR

SEQ ID NO: 76 - protein, 18 aa, Artificial Sequence
  GGCLHPFLPIRSVHFCPF
GGCLHPFLPIRSVHFCPF

SEQ ID NO: 77 - protein, 18 aa, Artificial Sequence
  C4m#19-5th4w-NN6-25
GGCLHPVLPIRSRHFCPV

SEQ ID NO: 78 - protein, 18 aa, Artificial Sequence
  C4m#19-5th4w-NN6-21
GGCWHPWLPILSPHFCPR

SEQ ID NO: 79 - protein, 18 aa, Artificial Sequence
  C4m#19-5th4w-NN6-39
GGCWHPFLPIRSLHFCPR

SEQ ID NO: 80 - protein, 18 aa, Artificial Sequence
  C4m#19-5th4w-NN6-70
GGCMHPFLPIRSMHFCPL

SEQ ID NO: 81 - protein, 18 aa, Artificial Sequence
  C4m#19-5th2w-NNS6-14
GGCWHPFLPIRSYHFCPW

SEQ ID NO: 82 - protein, 18 aa, Artificial Sequence
  C4m#19-5th4w-NN6-47
GGCPHPWLPIFSRHFCPV

SEQ ID NO: 83 - protein, 18 aa, Artificial Sequence
  C4m#19-5th4w-NN6-54
GGCLHPGLPIRSLHFCPR

SEQ ID NO: 84 - protein, 18 aa, Artificial Sequence
  C4m#19-5th4w-NN6-81
GGCFHPYLPIRSWHFCSR

SEQ ID NO: 85 - protein, 18 aa, Artificial Sequence
  C4m#19-5th4w-NN6-53
GGCRHPFLPIYSVHFCAC

SEQ ID NO: 86 - protein, 18 aa, Artificial Sequence
  C4m#19-5th4w-NN6-34
GGCFHPFLPIRSDHFCSR

SEQ ID NO: 87 - protein, 18 aa, Artificial Sequence
  C4m#19-5th4w-NN6-4
GGCRHPYLPIVSRHFCPF

SEQ ID NO: 88 - protein, 18 aa, Artificial Sequence
  C4m#19-5th2w-NNS6-21
GGCWHPFLPIRSSHFCPS

SEQ ID NO: 89 - protein, 18 aa, Artificial Sequence
  C4m#19-5th4w-NN6-20
GGCSHPQLPILSDHFCSA

SEQ ID NO: 90 - protein, 18 aa, Artificial Sequence
  C4m#19-5th4w-NN6-77
GGCRHPFLPIVSTHFCSR

SEQ ID NO: 91 - protein, 18 aa, Artificial Sequence
  C4m#19-5th4w-NN6-23
GGCRHPHLPIWSWHFCGR

SEQ ID NO: 92 - protein, 18 aa, Artificial Sequence
  C4m#19-5th4w-NN6-44
GGCRHPFLPIVSWHFCGR

SEQ ID NO: 93 - protein, 18 aa, Artificial Sequence
  C4m#19-5th4w-NN6-92-1
GGCVHPFLPIRSVHFCAL

SEQ ID NO: 94 - protein, 18 aa, Artificial Sequence
  C4m#19-5th4w-NN6-91
GGCMHPYLRIISPHFCRG

SEQ ID NO: 95 - protein, 18 aa, Artificial Sequence
  C4m#19-5th4w-NN6-19
GGCRHPYLPIWSIHFCQK

SEQ ID NO: 96 - protein, 18 aa, Artificial Sequence
  C4m#19-5th4w-NN6-55-1
GGCWHPFLPISSPHFCGR

SEQ ID NO: 97 - protein, 18 aa, Artificial Sequence
  C4m#19-5th4w-NN6-55-4
GGCVHPSLPILSVHFCPR

SEQ ID NO: 98 - protein, 18 aa, Artificial Sequence
  C4m#19-5th2w-NNS6-22
GGCMHPFLPIQSVHFCSR

SEQ ID NO: 99 - protein, 18 aa, Artificial Sequence
  C4m#19-5th4w-NN6-93
GGCCHPWLPIVSTHFCGL

SEQ ID NO: 100 - protein, 18 aa, Artificial Sequence
  C4m#19-5th4w-NN6-9
GGCFHPYLPIRSWHFCGR

SEQ ID NO: 101 - protein, 18 aa, Artificial Sequence
  C4m#19-5th4w-NN6-52
GGCLHPFLPIRSVHFCGC

SEQ ID NO: 102 - protein, 18 aa, Artificial Sequence
  C4m#19-5th2w-NNS6-55
GGCMHPYLPIKSVHFCPL

SEQ ID NO: 103 - protein, 18 aa, Artificial Sequence
  C4m#19-5th4w-NN6-82
GGCFHPFLPIRSLHFCCR

SEQ ID NO: 104 - protein, 18 aa, Artificial Sequence
  C4m#19-5th4w-NN6-50
GGCKHPVLPIWSWHFCRI

SEQ ID NO: 105 - protein, 18 aa, Artificial Sequence
  C4m#19-5th4w-NN6-78
GGCWHPALPIRSWHFCPR

SEQ ID NO: 106 - protein, 18 aa, Artificial Sequence
  C4m#19-5th4w-NN6-57
GGCMHPYLPIVSVHFCPC

SEQ ID NO: 107 - protein, 18 aa, Artificial Sequence
  C4m#19-5th4w-NN6-59
GGCLHPHLPIWSLHFCPL

SEQ ID NO: 108 - protein, 18 aa, Artificial Sequence
  C4m#19-5th4w-NN6-24
GGCWHPWLPIYSHHFCPI

SEQ ID NO: 109 - protein, 18 aa, Artificial Sequence
  C4m#19-5th4w-NN6-32
GGCPHPFLPIWSVHFCPF

SEQ ID NO: 110 - protein, 18 aa, Artificial Sequence
  C4m#19-5th4w-NN6-56
GGCWHPFLPIQSLHFCGV

SEQ ID NO: 111 - protein, 18 aa, Artificial Sequence
  C4m#19-5th2w-NNS6-67
GGCQHPWLPISSWHFCPR

SEQ ID NO: 112 - protein, 18 aa, Artificial Sequence
  C4m#19-5th4w-NN6-69-1
GGCSHPFLPILSVHFCPF

SEQ ID NO: 113 - protein, 18 aa, Artificial Sequence
  C4m#19-5th4w-NN6-69-3
GGCLHPWLPIVSVHFCAR

SEQ ID NO: 114 - protein, 18 aa, Artificial Sequence
  C4m#19-5th4w-NN6-79
GGCWHPVLPIRSCHFCAV

SEQ ID NO: 115 - protein, 18 aa, Artificial Sequence
  C4m#19-5th4w-NN6-68
GGCLHPWLPIRSRHFCSR

SEQ ID NO: 116 - protein, 18 aa, Artificial Sequence
  C4m3127-4th-NNS6-28
GGCLHPFLPIFSSHFCAL

SEQ ID NO: 117 - protein, 18 aa, Artificial Sequence
  C4m#19-5th4w-NN6-7
GGCRHPFLPIWSWHFCPF

SEQ ID NO: 118 - protein, 18 aa, Artificial Sequence
  C4m#19-5th4w-NN6-85
GGCWHPFLPIFSVHFCGC

SEQ ID NO: 119 - protein, 18 aa, Artificial Sequence
  C4m#19-5th2w-NNS6-71
GGCRHPYLPIASPHFCPG

SEQ ID NO: 120 - protein, 18 aa, Artificial Sequence
  C4m3127-4th-NNS6-90
GGCKHPVLPIVSLHFCPW

SEQ ID NO: 121 - protein, 18 aa, Artificial Sequence
  C4m#19-5th4w-NN6-37
GGCLHPFLPISSWHFCGR

SEQ ID NO: 122 - protein, 18 aa, Artificial Sequence
  C4m#19-5th2w-NNS6-94
GGCFHPYLPISSVHFCGR

SEQ ID NO: 123 - protein, 18 aa, Artificial Sequence
  C4m#19-5th4w-NN6-51
GGCWHPYLPIVSWHFCSR

SEQ ID NO: 124 - protein, 18 aa, Artificial Sequence
  C4m#19-5th4w-NN6-11
GGCRHPFLPIWSHHFCVC

SEQ ID NO: 125 - protein, 18 aa, Artificial Sequence
  C4m#19-5th4w-NN6-87
GGCLHPVLPIRSFHFCWR

SEQ ID NO: 126 - protein, 18 aa, Artificial Sequence
  C4m3127-4th-NNS6-87
GGCRHPWLPIVSWHFCSR

SEQ ID NO: 127 - protein, 18 aa, Artificial Sequence
  C4m#19-5th4w-NN6-90
GGCRHPWLPIVSYHFCSS

SEQ ID NO: 128 - protein, 18 aa, Artificial Sequence
  C4m#19-5th2w-NNS6-25
GGCKHPFLPISSHHFCPY

SEQ ID NO: 129 - protein, 18 aa, Artificial Sequence
  C4m3127-4th-NNS6-83
GGCRHPFLPIFSKHFCEL

SEQ ID NO: 130 - protein, 18 aa, Artificial Sequence
  C4m3127-4th-NNS6-17
GGCRHPVLPIVSLHFCPG

SEQ ID NO: 131 - protein, 18 aa, Artificial Sequence
  C4m#19-5th2w-NNS6-12
GGCWHPYLPIRSVHFCGR

SEQ ID NO: 132 - protein, 18 aa, Artificial Sequence
  C4m3127-4th-NNS6-72
GGCVHPYLPISSIHFCSM

SEQ ID NO: 133 - protein, 18 aa, Artificial Sequence
  C4m#19-5th2w-NNS6-90
GGCRHPFLPIVSFHFCPF

SEQ ID NO: 134 - protein, 18 aa, Artificial Sequence
  C4m#19-5th4w-NN6-86
GGCAHPVLPIRSLHFCPH

SEQ ID NO: 135 - protein, 18 aa, Artificial Sequence
  C4m#19-5th4w-NN6-58
GGCRHPFLPISSIHFCCI

SEQ ID NO: 136 - protein, 18 aa, Artificial Sequence
  C4m#19-5th2w-NNS6-3
GGCAHPFLPIFSPHFCPF

SEQ ID NO: 137 - protein, 18 aa, Artificial Sequence
  C4m#19-5th4w-NN6-2
GGCWHPHLPIVSSHFCLF

SEQ ID NO: 138 - protein, 18 aa, Artificial Sequence
  C4m#19-5th4w-NN6-71
GGCPHPWLPIASVHFCPF

SEQ ID NO: 139 - protein, 18 aa, Artificial Sequence
  C4m#19-5th4w-NN6-62
GGCQHPYLPIWSFHFCPF

SEQ ID NO: 140 - protein, 18 aa, Artificial Sequence
  C4m#19-5th4w-NN6-30
GGCWHPFLPIMSFHFCPY

SEQ ID NO: 141 - protein, 18 aa, Artificial Sequence
  C4m#19-5th4w-NN6-38
GGCSHPFLPIFSLHFCPV

SEQ ID NO: 142 - protein, 18 aa, Artificial Sequence
  C4m#19-5th2w-NNS6-8
GGCRHPFLPIFSLHFCAR

SEQ ID NO: 143 - protein, 18 aa, Artificial Sequence
  C4m3127-4th-NNS6-16
GGCWHPYLPIFSVHFCPR

SEQ ID NO: 144 - protein, 18 aa, Artificial Sequence
  C4m#19-5th4w-NN6-63
GGCWHPYLPILSRHFCQA

SEQ ID NO: 145 - protein, 18 aa, Artificial Sequence
  C4m#19-5th4w-NN6-14
GGCWHPFLPIRSVHFCCR

SEQ ID NO: 146 - protein, 18 aa, Artificial Sequence
  C4m#19-5th4w-NN6-1
GGCRHPYLPIFSAHFCRS

SEQ ID NO: 147 - protein, 18 aa, Artificial Sequence
  C4m#19-5th4w-NN6-6
GGCMHPFLPIKSSHFCRK

SEQ ID NO: 148 - protein, 18 aa, Artificial Sequence
  C4m#19-5th2w-NNS6-51
GGCRHPFLPIVSLHFCPF

SEQ ID NO: 149 - protein, 18 aa, Artificial Sequence
  C4m#19-5th4w-NN6-45
GGCRHPWLPIWSVHFCGV

SEQ ID NO: 150 - protein, 18 aa, Artificial Sequence
  C4m3127-4th-NNS6-12
GGCSHPFLPIRSRHFCPF

SEQ ID NO: 151 - protein, 18 aa, Artificial Sequence
  C4m#19-5th2w-NNS6-75
GGCWHPWLPIKSPHFCAL

SEQ ID NO: 152 - protein, 18 aa, Artificial Sequence
  C4m#19-5th4w-NN6-74
GGCFHPFLPIYSWHFCAR

SEQ ID NO: 153 - protein, 18 aa, Artificial Sequence
  C4m#19-5th4w-NN6-5-1
GGCWHPYLPIQSLHFCPF

SEQ ID NO: 154 - protein, 18 aa, Artificial Sequence
  C4m#19-5th4w-NN6-5-3
GGCWHPFLPILSLHFCGW

SEQ ID NO: 155 - protein, 18 aa, Artificial Sequence
  C4m#19-5th4w-NN6-46-1
GGCVHPALPIWSFHFCPS

SEQ ID NO: 156 - protein, 18 aa, Artificial Sequence
  C4m#19-5th4w-NN6-64
GGCWHPVLPIRSVHFCCR

SEQ ID NO: 157 - protein, 18 aa, Artificial Sequence
  C4m3127-4th-NNS6-48
GGCFHPWLPIASWHFCGR

SEQ ID NO: 158 - protein, 18 aa, Artificial Sequence
  C4m3127-4th-NNS6-64
GGCLHPYLPIVSSHFCPF

SEQ ID NO: 159 - protein, 18 aa, Artificial Sequence
  C4m3127-4th-NNS6-80
GGCGHPFLPIFSLHFCPR

SEQ ID NO: 160 - protein, 18 aa, Artificial Sequence
  C4m3127-4th-NNS6-79
GGCLHPFLPIRSVHFCLR

SEQ ID NO: 161 - protein, 18 aa, Artificial Sequence
  C4m3127-4th-NNS6-2
GGCWHPSLPIYSPHFCPL

SEQ ID NO: 162 - protein, 18 aa, Artificial Sequence
  C4m#19-5th4w-NN6-65
GGCWHPFLPILSLHFCSR

SEQ ID NO: 163 - protein, 18 aa, Artificial Sequence
  C4m#19-5th4w-NN6-83
GGCLHPLLPILSVHFCPS

SEQ ID NO: 164 - protein, 18 aa, Artificial Sequence
  C4m3127-4th-NNS6-60
GGCWHPFLPISSWHFCGL

SEQ ID NO: 165 - protein, 18 aa, Artificial Sequence
  C4m#19-5th2w-NNS6-53
GGCLHPWLPIRSFHFCGA

SEQ ID NO: 166 - protein, 18 aa, Artificial Sequence
  C4m#19-5th2w-NNS6-45
GGCRHPFLPIWSVHFCWR

SEQ ID NO: 167 - protein, 18 aa, Artificial Sequence
  C4m3127-4th-NNS6-3
GGCSHPFLPIWSVHFCGQ

SEQ ID NO: 168 - protein, 18 aa, Artificial Sequence
  C4m#19-5th2w-NNS6-6
GGCWHPYLPILSLHFCQR

SEQ ID NO: 169 - protein, 18 aa, Artificial Sequence
  C4m#19-5th2w-NNS6-27
GGCFHPFLPIRSCHFCAR

SEQ ID NO: 170 - protein, 18 aa, Artificial Sequence
  C4m#19-5th2w-NNS6-37
GGCIHPFLPIWSIHFCSR

SEQ ID NO: 171 - protein, 18 aa, Artificial Sequence
  C4m#19-5th2w-NNS6-33
GGCRHPFLPIWSFHFCWG

SEQ ID NO: 172 - protein, 18 aa, Artificial Sequence
  C4m#19-5th2w-NNS6-79
GGCRHPFLPIVSRHFCVA

SEQ ID NO: 173 - protein, 18 aa, Artificial Sequence
  C4m3127-4th-NNS6-85
GGCGHPFLPIFSSHFCPL

SEQ ID NO: 174 - protein, 18 aa, Artificial Sequence
  C4m#19-5th2w-NNS6-30
GGCWHPFLPILSWHFCGR

SEQ ID NO: 175 - protein, 18 aa, Artificial Sequence
  C4m#19-5th4w-NN6-89
GGCMHPFLPIISQHFCAF

SEQ ID NO: 176 - protein, 18 aa, Artificial Sequence
  C4m#19-5th2w-NNS6-86
GGCFHPYLPIWSLHFCRS

SEQ ID NO: 177 - protein, 18 aa, Artificial Sequence
  C4m3127-4th-NNS6-30
GGCWHPCLPILSPHFCPR

SEQ ID NO: 178 - protein, 18 aa, Artificial Sequence
  C4m3127-4th-NNS6-26
GGCAHPALPIWSCHFCPF

SEQ ID NO: 179 - protein, 18 aa, Artificial Sequence
  C4m#19-5th2w-NNS6-74
GGCMHPFLPIRSWHFCSR

SEQ ID NO: 180 - protein, 18 aa, Artificial Sequence
  C4m3127-4th-NNS6-89
GGCWHPFLPIFSAHFCMR

SEQ ID NO: 181 - protein, 18 aa, Artificial Sequence
  C4m#19-5th2w-NNS6-84
GGCWHPFLPIVSFHFCPF

SEQ ID NO: 182 - protein, 18 aa, Artificial Sequence
  C4m#19-5th2w-NNS6-28
GGCAHPYLPIRSVHFCPC

SEQ ID NO: 183 - protein, 18 aa, Artificial Sequence
  C4m#19-5th2w-NNS6-4
GGCFHPWLPISSLHFCPF

SEQ ID NO: 184 - protein, 18 aa, Artificial Sequence
  C4m#19-5th2w-NNS6-46
GGCFHPFLPIFSYHFCGL

SEQ ID NO: 185 - protein, 18 aa, Artificial Sequence
  C4m#19-5th4w-NN6-61
GGCPHPMLPIFSVHFCPY

SEQ ID NO: 186 - protein, 18 aa, Artificial Sequence
  C4m3127-4th-NNS6-94
GGCRHPVLPIWSSHFCPW

SEQ ID NO: 187 - protein, 18 aa, Artificial Sequence
  C4m#19-5th2w-NNS6-26
GGCLHPFLPIVSIHFCSA

SEQ ID NO: 188 - protein, 18 aa, Artificial Sequence
  C4m#19-5th4w-NN6-36
GGCRHPYLPIWSAHFCCR

SEQ ID NO: 189 - protein, 18 aa, Artificial Sequence
  C4m#19-5th4w-NN6-75
GGCWHPFLPIKSVHFCCA

SEQ ID NO: 190 - protein, 18 aa, Artificial Sequence
  C4m#19-5th2w-NNS6-70
GGCRHPFLPIVSWHFCAA

SEQ ID NO: 191 - protein, 18 aa, Artificial Sequence
  C4m#19-5th2w-NNS6-34
GGCCHPFLPIYSVHFCAS

SEQ ID NO: 192 - protein, 18 aa, Artificial Sequence
  C4m#19-5th4w-NN6-94
GGCLHPFLPIYSLHFCPC

SEQ ID NO: 193 - protein, 18 aa, Artificial Sequence
  C4m3127-4th-NNS6-66
GGCAHPHLPIRSVHFCSF

SEQ ID NO: 194 - protein, 18 aa, Artificial Sequence
  C4m#19-5th2w-NNS6-43
GGCRHPFLPIWSSHFCAA

SEQ ID NO: 195 - protein, 18 aa, Artificial Sequence
  C4m3127-4th-NNS6-7
GGCRHPFLPIRSPHFCGS

SEQ ID NO: 196 - protein, 18 aa, Artificial Sequence
  C4m#19-5th4w-NN6-72
GGCWHPWLPIVSVHFCGC

SEQ ID NO: 197 - protein, 18 aa, Artificial Sequence
  C4m3127-4th-NNS6-70
GGCSHPYLPIHSRHFCLS

SEQ ID NO: 198 - protein, 18 aa, Artificial Sequence
  C4m#19-5th2w-NNS6-68
GGCLHPWLPIVSCHFCPR

SEQ ID NO: 199 - protein, 18 aa, Artificial Sequence
  C4m#19-5th2w-NNS6-87
GGCLHPYLPIFSVHFCAL

SEQ ID NO: 200 - protein, 18 aa, Artificial Sequence
  C4m#19-5th2w-NNS6-66
GGCRHPFLPIFSVHFCPF

SEQ ID NO: 201 - protein, 18 aa, Artificial Sequence
  C4m3127-4th-NNS6-20
GGCLHPFLPILSLHFCPF

SEQ ID NO: 202 - protein, 18 aa, Artificial Sequence
  C4m#19-5th2w-NNS6-61
GGCCHPWLPIRSVHFCPF

SEQ ID NO: 203 - protein, 18 aa, Artificial Sequence
  C4m3127-4th-NNS6-19
GGCMHPSLPIWSWHFCPH

SEQ ID NO: 204 - protein, 18 aa, Artificial Sequence
  C4m#19-5th4w-NN6-40
GGCFHPFLPIRSHHFCML

SEQ ID NO: 205 - protein, 18 aa, Artificial Sequence
  C4m#19-5th4w-NN6-49
GGCMHPYLPIRSFHFCSC

SEQ ID NO: 206 - protein, 18 aa, Artificial Sequence
  C4m#19-5th4w-NN6-80
GGCRHPFLPIFSWHFCAV

SEQ ID NO: 207 - protein, 18 aa, Artificial Sequence
  C4m#19-5th2w-NNS6-80
GGCLHPFLPIVSWHFCPC

SEQ ID NO: 208 - protein, 18 aa, Artificial Sequence
  C4m3127-4th-NNS6-37
GGCWHPVLPIRSIHFCGC

SEQ ID NO: 209 - protein, 18 aa, Artificial Sequence
  C4m#19-5th2w-NNS6-93
GGCFHPFLPIQSRHFCCR

SEQ ID NO: 210 - protein, 18 aa, Artificial Sequence
  C4m3127-4th-NNS6-95
GGCWHPVLPIRSGHFCSR

SEQ ID NO: 211 - protein, 18 aa, Artificial Sequence
  C4m#19-5th2w-NNS6-41
GGCWHPVLPIRSDHFCCR

(19) United States
(12) Patent Application Publication
(10) Pub. No.: US 2024/0092836 A1
(43) Pub. Date: Mar. 21, 2024

(54) CYCLIC PEPTIDE HAVING CTLA-4 INHIBITORY ACTIVITY AND USE THEREOF

(71) Applicant: GeneFrontier Corporation, Kashiwa-shi, Chiba (JP)

(72) Inventors: Kanehisa KOJOH, Kashiwa (JP); Kumiko TSUIHIJI, Kashiwa (JP); Shizue KATOH, Kashiwa (JP); Mikiko NAKAMURA, Kashiwa (JP)

(73) Assignee: GeneFrontier Corporation, Kashiwa-shi, Chiba (JP)

(21) Appl. No.: 17/767,550

(22) PCT Filed: Sep. 30, 2020
(86) PCT No.: PCT/JP2020/037100

§ 371 Date: Apr. 8, 2022

(30) Foreign Application Priority Data
Oct. 8, 2019 (JP) 2019-185266

(51) Int. Cl. C07K 7/64

(52) U.S. Cl. CPC C07K 7/64

(57) ABSTRACT

The present invention provides a cyclic peptide which comprises the amino acid sequence represented by formula (I)

X₁-His-Pro-X₄-Leu-X₆-X₇-X₈-Ser-X₁₀-His-Phe (I)
in the cycle and has an activity to specifically bind to human CTLA-4, wherein X₁, X₄, X₆, X₇, X₈ and X₁₀ are each independently any amino acid.

### TECHNICAL FIELD

The present invention relates to a cyclic peptide that inhibits the binding of CTLA-4 to CD80/CD86, and uses thereof.

### BACKGROUND ART

Small molecule drugs, which have been the core of conventional pharmaceuticals, have a problem that, for example, they are prone to cause toxicity and side effects due to their low specificity for the target molecule, and they are unsuitable for inhibiting protein-protein interactions due to their small molecular size. In addition, antibody drugs, which are developed by many pharmaceutical companies in recent years, have a problem that the application to intracellular target molecules is difficult because they do not easily enter cells, they are difficult to be administered orally, and manufacturing cost is high. Therefore, middle molecule drugs based on peptides are being developed recently, as an ideal drug that compensates for the problems of both small molecule drugs and antibody drugs (Non-Patent Document 1).

Cyclic peptides are known to have superior specificity to target molecules, stability, and membrane permeability than linear peptides (Non-Patent Document 2). Cyclic peptides with desired activity can be efficiently obtained by in vitro selection experiments such as phage display (Non-Patent Document 3), mRNA display (Non-Patent Documents 4 and 5) or the like. Cyclic peptides with desired activity can also be prepared enzymatically and selected in a cell (Non-Patent Document 6).

Ribosome display (Patent Document 1) is one of the in vitro selection methods, as well as phage display and mRNA display. Recently, the inventors have developed a novel ribosome display system using the PUREsystem, a cell-free translation system in which factors required for translation are individually prepared and reconstituted, and further optimized for ribosome display (Patent Document 2). In this ribosome display system, the concentration of oxidants and reductants optimal for S—S bond formation can be freely set, and various molecular chaperones (e.g., DsbC, PDI, GroEL-ES, DnaK and the like) can be added or combined.

Immune checkpoint molecules such as CTLA-4 (cytotoxic T-lymphocyte-associated protein 4) and PD-1 (programmed death 1) are negative regulators of T cell immune function. Inhibition of the function of these molecules results in activation of the immune system and is clinically applied as a therapeutic drug. As for CTLA-4, it was shown that an anti-CTLA-4 antibody inhibits the interaction between CTLA-4 and CD80/CD86 and suppresses cancer growth (Non-Patent Document 7). However, no middle-molecule drugs that inhibit CTLA-4 function have yet been developed.

### DOCUMENT LIST

### Patent Documents

Patent Document 1: JP2008-271903A
Patent Document 2: US patent application publication No. 2012/231496 specification

### Non-Patent Documents

Non-Patent Document 1: Zorzi, A. et al., Curr. Opin. Chem. Biol., (2017),38, 24-29
Non-Patent Document 2: Roxin, A. and Zheng, G., Future Med. Chem., (2012), 4(12), 1601-1618
Non-Patent Document 3: Desimmie, B. A. et al., Molecular Therapy, (2012), 20(11), 2064-2075
Non-Patent Document 4: Guillen Schlippe, Y. V. et al., J. Am. Chem. Soc., (2012), 134, 10469-10477
Non-Patent Document 5: Bashiruddin, N. K. and Suga, H., Curr. Opin. Chem. Biol., (2015), 24, 131-138
Non-Patent Document 6: Tavassoli, A., Curr. Opin. Chem. Biol., (2017),38, 30-35
Non-Patent Document 7: Leach, D. R. et al., Science, (1996), 271(5256), 1734-1736

### SUMMARY OF THE INVENTION

### Problems to be Solved by the Invention

The purpose of the present invention is to provide a cyclic peptide that inhibit CTLA-4 function.

### Means of Solving the Problems

The present inventors succeeded in obtaining multiple cyclic peptides that bind specifically to CTLA-4 by performing selection experiments by ribosome display under a condition optimized for S—S bond formation between two cysteines in a cyclic peptide. These cyclic peptides inhibited the interaction between CTLA-4 and CD80. Affinity Maturation was performed to obtain many high-affinity CTLA-4 binders. Furthermore, Alanine scanning was performed to successively identify the minimal amino acid residues required for binding to CTLA-4. Based on these findings, the present inventors have investigated further and completed the present invention.

Accordingly, the present invention relates to the followings.

[1] A cyclic peptide which comprises the amino acid sequence represented by formula (I):

X₁-His-Pro-X₄-Leu-X₆-X₇-X₈-Ser-X₁₀-His-Phe (I)
in the cycle and has an activity to specifically bind to human CTLA-4, wherein X₁, X₄, X₆, X₇, X₈ and X₁₀ are each independently any amino acid.

[2] The cyclic peptide according to [1], wherein the cyclic peptide comprises the amino acid sequence represented by formula (II):

X₀-X₁-His-Pro-X₄-Leu-X₆-X₇-X₈-Ser-X₁₀-His-Phe-X₀₀ (II)
wherein X₁, X₄, X₆, X₇, X₈ and X₁₀ are synonymous with those defined in formula (I), X₀ and X₀₀ are each independently any amino acid, and
wherein the peptide is cyclized by an intramolecular bond between X₀ and X₀₀.

[3] The cyclic peptide according to [2],
wherein the cyclic peptide comprises the amino acid sequence represented by formula (III):

(Y)m-X₀-X₁-His-Pro-X₄-Leu-X₆-X₇-X₈-Ser-X₁₀-His-Phe-X₀₀-(Z)n (III)
wherein X₀, X₁, X₄, X₆, X₇, X₈, X₁₀ and X₀₀ are synonymous with those defined in formula (II),
(Y)m is an amino acid sequence having a length of m amino acid(s),
(Z)n is an amino acid sequence having a length of n amino acid(s),
m is any integer selected from the group consisting of 0, 1 and 2, and
n is any integer selected from the group consisting of 0, 1 and 2.

[4] The cyclic peptide according to [2] or [3],
wherein X₀ and X₀₀ are each independently an amino acid having a side chain containing a thiol group.

[5] The cyclic peptide according to [4],
wherein X₀ is Cys, and
X₀₀ is Cys.

[6] The cyclic peptide according to [4] or [5],
wherein the peptide is cyclized by an intramolecular disulfide bond between a thiol group in the side chain of X₀ and a thiol group in the side chain of X₀₀.

[7] The cyclic peptide according to any of [1] to [6],
wherein X₆ is Leu, Pro, Gln, Lys or Arg, and
X₇ is Val, Leu, Ile or Thr.

[8] The cyclic peptide according to [7],
wherein X₆ is Pro, and
X₇ is Ile.

[9] The cyclic peptide according to any of [3] to [8], wherein n is 2,
(Z)n consists of an amino acid sequence represented by X₊₁-X₊₂, and X₊₁ and X₊₂ are each independently any amino acid.

[10] The cyclic peptide according to any of [3] to [9], wherein m is 2,
(Y)m consists of an amino acid sequence represented by X₋₂-X₋₁, and X₋₂ and X₋₁ are each independently any amino acid.

[11] The cyclic peptide according to [10], Wherein X₋₂ is Gly, and X₋₁ is Gly, Ser or Asp.

[12] The cyclic peptide according to any of [1] to [11], wherein the amino acid sequence represented by formula (I) consists of
(1a) an amino acid sequence from position 4 to position 15 in the amino acid sequence represented by any of SEQ ID NOs: 30-211, or
(2a) an amino acid sequence having a substitution at least at one amino acid corresponding to one selected from X₁, X₄, X₆, X₇, X₈ and X₁₀ in an amino acid sequence from position 4 to position 15 in the amino acid sequence represented by any of SEQ ID NOs: 30-211.

[13] The cyclic peptide according to any of [2] to [12], wherein the amino acid sequence represented by formula (II) consists of
(1b) an amino acid sequence from position 3 to position 16 in the amino acid sequence represented by any of SEQ ID NOs: 30-211, or
(2b) an amino acid sequence having a substitution at least at one amino acid corresponding to one selected from X₁, X₄, X₆, X₇, X₈ and X₁₀ in an amino acid sequence from position 3 to position 16 in the amino acid sequence represented by any of SEQ ID NOs: 30-211

[14] The cyclic peptide according to any of [3] to [13], wherein the amino acid sequence represented by formula (III) consists of
(1c) the amino acid sequence represented by any of SEQ ID NOs: 30-211, or
(2c) an amino acid sequence having a substitution at least at one amino acid corresponding to one selected from X₋₂, X₋₁, X₁, X₄, X₆, X₇, X₈, X₁₀, X₊₁ and X₊₂ in the amino acid sequence represented by any of SEQ ID NOs: 30-211.

[15] The cyclic peptide according to any of [1] to [14], wherein the cyclic peptide has an activity to inhibit the interaction between human CTLA-4 and human CD80.

[16] A pharmaceutical composition which comprises the cyclic peptide according to any of [1] to [14] and a pharmaceutically acceptable carrier or excipient.

[17] The pharmaceutical composition according to [16], which is for inhibiting CTLA-4.

[18] The pharmaceutical composition according to [16], which is for activating T cells.

[19] The pharmaceutical composition according to [16], which is for preventing or treating a tumor.

[20] A CTLA-4 inhibitor, which comprises the cyclic peptide according to any of [1] to [15].

[21] A method for preventing or treating a tumor in a subject, which comprises administering an effective amount of the cyclic peptide according to any of [1] to [15] to the subject.

[22] The cyclic peptide according to any of [1] to [15], which is for use in preventing or treating a tumor.

[23] Use of the cyclic peptide according to any of [1] to [15], for manufacturing a pharmaceutical composition for preventing or treating a tumor.

### Effect of the Invention

According to the present invention, a cyclic peptide which inhibits CTLA-4 function is provided. The cyclic peptide of the present invention is useful as an immune checkpoint inhibitor and expected to be applied for a prophylactic or therapeutic drug against diseases (e.g., cancer) which can be treated or prevented by inhibiting CTLA-4 function.

### BRIEF DESCRIPTION OF THE DRAWINGS

FIG. 1 Sequence information of the cyclic peptide random library is shown. 5′FLAG tag sequence and 3′c-Myc tag sequence are indicated by a rectangle frame, and a random sequence (NNS) is indicated by italic type. Two cysteine residues are underlined. GG Linker means a linker consisting of two glycine residues.

FIG. 2 A schematic of pET-Mal vector is shown. MBP fusion protein-expression vector was constructed by inserting a cyclic peptide gene at EcoRI-HindIII site located downstream of the MBP gene.

FIG. 3 The results of the selection experiment by ribosome display against CTLA-4-Fc are shown. The amount of recovered mRNA got increased with each selection round.

FIG. 4 The results of the ribosome display pull down assay are shown. All clones except No. 2 are clones specifically binding to Fc moiety, since they bound to both CTLA-4 Fc and Human Fc.

FIG. 5 The results of measured affinity of synthetic peptides to CTLA-4 expressed on the cell surface analyzed by FACS are shown.

FIG. 6 The results of the alanine scanning are shown.

### MODE FOR CARRYING OUT THE INVENTION

### I. Definition

### <Peptide>

As used herein, the terms “peptide”, “oligopeptide”, “polypeptide” and “protein” are used interchangeably and mean a polymer of amino acids of any length. The polymer may be linear, branched or cyclic.

### <Amino Acid>

As used herein, the term “amino acid” is used in the broadest sense and encompasses not only natural amino acids but also artificial amino acid variants or derivatives thereof. For example, natural proteinogenic amino acid, non-proteinogenic amino acid, and chemically synthesized compounds having properties known in the art as characteristics of an amino acid are encompassed within the term “amino acid”. Although each amino acid may have L-form or D-form configuration as appropriate, typically L-form configuration is used for all amino acids. The amino acids may be represented by a commonly used three-letter or single-letter abbreviation. Except when preceded by “D”, an amino acid refers to an L-amino acid. When the position of the amino group substitution is not particularly specified, an amino acid refers to an α-amino acid. L-amino acids are indicated by a single capital letter or the first capital letter in the three-letter symbol, and D-amino acids are indicated by a single lowercase letter or a lowercase three-letter symbol.

Natural proteinogenic amino acids include alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp) and tyrosine (Y or Tyr).

Non-proteinogenic amino acids refer to natural or non-natural amino acids other than natural proteinogenic amino acids. As examples of the non-proteinogenic amino acid, the followings can be exemplified:

### 1. Isomers of Natural Proteinogenic Amino Acids

### (1) Enantiomers (D-Amino Acids)

For example, D-alanine (a or ala), D-cysteine (c or cys), D-aspartic acid (d or asp), D-glutamic acid (e or glu), D-phenylalanine (f or phe), D-histidine (h or his), D-isoleucine (i or ile), D-lysine (k or lys), D-leucine (l or leu), D-methionine (m or met), D-asparagine (n or asn), D-proline (p or pro), D-glutamine (q or gln), D-arginine (r or arg), D-serine (s or ser), D-threonine (t or thr), D-valine (v or val), D-tryptophan (w or trp) and D-tyrosine (y or tyr) can be mentioned.

### (2) Diastereomers

For example, allothreonine (alThr) (L-form or D-form) and alloisoleucine (AIle) (L-form or D-form) can be mentioned.

(3) Amino acids having an amino group on a carbon other than α-position (β-amino acids, γ-amino acids or the like)

For example, β-alanine (bAla), β-leucine (bLeu), β-methionine (bMet), β-phenylalanine (bPhe), β-tyrosine (bTyr), β-cysteine (bCys), β-serine (bSer), β-threonine (bThr), β-asparagine (bAsn), β-glutamine (bGln), β-aspartic acid (bAsp), β-glutamic acid (bGlu), β-lysine (bLys), β-arginine (bArg), β-histidine (bHis) can be mentioned.

### (4) Amino Acids Peptide-Bonded at the β-Position of Carboxylic Acid

For example, isoaspartic acid (iAsp) and isoglutamic acid (iGlu) can be mentioned.

### (5) Side Chain Isomers

For example, norleucine (Nle), tert-leucine (Tle) and norvaline (Nva) can be mentioned.

### 2. Substituted Natural Proteinogenic Amino Acids

### (1) Amino Acids Having an Additional Alkylene Group in the Main Chain

The alkylene group is preferably a linear alkylene group having 1 to 4 carbon atoms. In the case of the alkylene group having one carbon atom (i.e., methylene group), the substituted natural proteinogenic amino acid may be a β-homo amino acid. Examples of the β-homo amino acid include β-homoalanine (Bha), β-homovaline (Bhv), β-homoleucine (Bhl), β-homoisoleucine (Bhi), β-homomethionine (Bhm), β-homophenylalanine (Bhf), β-homotryptophane (Bhw), β-homotyrosine (Bhy), β-homocysteine (Bhc), β-homoserine (Bhs), β-homothreonine (Bht), β-homoasparagine (Bhn), β-homoglutamine (Bhq), β-homoaspartic acid (Bhe), β-homoglutamine acid (Bhe), β-homolysine (Bhk), β-homoarginine (Bhr), β-homohistidine (Bhh) or the like.

In the case of the alkylene group other than methylene group, 3-aminobutyric acid (3Abu), 4-aminobutyric acid (4Abu), 6-aminohexanoic acid (εAhx), 5-aminopentanoic acid (δAva) or the like can be exemplified.

### (2) Amino Acids Having a Substituent at a Nitrogen Atom in the Main Chain (N-Substituted Amino Acids)

Although the kinds of substituents are not particularly limited, an alkyl group and formyl group can be exemplified. The alkyl group is preferably a linear or branched alkyl group having 1 to 6 carbon atoms, and examples of the linear C1-6 alkyl group include methyl group, ethyl group, n-propyl group, n-butyl group, n-pentyl group and n-hexyl group. Examples of the branched C1-6 alkyl group include isopropyl, isobutyl, sec-butyl, tert-butyl, isopentyl, neo-pentyl, 1-ethylpropyl, isohexyl, 1,1-dimethylbutyl, 2,2-dimethylbutyl, 3,3-dimethylbutyl, 2-ethylbutyl or the like. The alkyl group may be substituted by amino group, hydroxy group, thiol group or the like.

Examples of the N-substituted amino acid include N-methylglycine (sarcosine), N-methylalanine (MeAla), N-methylvaline (MeVal), N-methylleucine (MeLeu), N-methylisoleucine (MeIle), N-methylmethionine (MeMet), N-methylproline (MePro), N-methylphenylalanine (MePhe), N-methyltryptophan (MeTyp), N-methyltyrosine (MeTyr), N-methylcysteine (MeCys), N-methylserine (MeSer), N-methylthreonine (MeThr), N-methylasparagine (MeAsn), N-methylglutamine (MeGln), N-methylaspartic acid (MeAsp), N-methylglutamic acid (MeGlu), N-methyllysine (MeLys), N-methylarginine (MeArg), N-methylhistidine (MeHis), N-ethylglycine (EtGly), N-propylglycine (PrGly), N-(4-aminobutyl)glycine, N-(4-aminopropyl)glycine, N-ethylasparagine (EtAsn), N-formylmethionine (FoMet) and N-mercaptoethylglycine (NHSEtGly).

### (3) Amino Acids Having an Additional Substituent at the α-Carbon (α-Substituted Amino Acids)

Although the kinds of substituents are not particularly limited, an alkyl group can be exemplified. The alkyl group is preferably a linear or branched alkyl group having 1 to 6 carbon atoms, and examples of the linear C1-6 alkyl group include methyl group, ethyl group, n-propyl group, n-butyl group, n-pentyl group and n-hexyl group. Examples of the branched C1-6 alkyl group include isopropyl, isobutyl, sec-butyl, tert-butyl, isopentyl, neo-pentyl, 1-ethylpropyl, isohexyl, 1,1-dimethylbutyl, 2,2-dimethylbutyl, 3,3-dimethylbutyl, 2-ethylbutyl or the like. The alkyl group may be substituted by amino group, hydroxy group, thiol group or the like. A number of substituent(s) on the α-carbon is 1 or 2, preferably 1. Alkyl groups on a disubstituted α-carbon may be C—C bonded each other to construct a cycloalkyl group. Preferably, the cycloalkyl group has 4 to 6 carbon atoms. Examples of the C4-6 cycloalkyl group include cyclopropyl group, cyclopentyl group and cyclohexyl group.

Examples of the α-substituted amino acid include α-aminoisobutyric acid (methylalanine) (Aib), α-methylleucine (Aml), α-methylproline (Amp), α,α-diethylglycine (Deg), α,α-dibutylglycine (Dbg), 1-amino-cyclohexane-1-carboxylic acid (Ac6c), 1-amino-cyclopentane-1-carboxylic acid (Ac5c), 1-amino-cyclopropane-1-carboxylic acid (Ac3c), 1-amino-(4-N-piperidinyl)carboxylic acid (Apc) or the like.

(4) Amino Acids Having a Side Chain with an Alkylene Chain of Different Length

This type of amino acids has a structure in which C1-C2 alkylene chain has been eliminated from the side chain of a natural proteinogenic amino acid, or C1-C6 linear alkylene group has been inserted between the side chain and the α-carbon. Among them, examples of the amino acid having a structure in which C1 alkylene group (i.e., methylene group) has been inserted between the side chain and the α-carbon of a natural proteinogenic amino acid are indicated below along with the corresponding natural proteinogenic amino acids.
<alanine>homoalanine (2-aminobutyric acid) (hAla), 2-aminopentanoic acid (2Apn)
<leucine>homoleucine (hLeu)
<isoleucine>homoisoleucine (hIle)
<methionine>homomethionine (hMet)
<proline>pipecolic acid (Pip), azetidine-2-carboxylic acid (Az2Ca), azetidine-3-carboxylic acid (Az3Ca)
<phenylalanine>homophenylalanine (hPhe), phenylglycine (fGly),
<tryptophan>homotryptophane (hTrp)
<tyrosine>homotyrosine (hTyr),
<cysteine>homocysteine (hCys), norcysteine (Ncy)
<serine>homoserine (hSer)
<threonine>homothreonine (hThr),
<glutamine>homoglutamine (hGln)
<glutamic acid>homoglutamine acid (hGlu), 2-aminoadipic acid (Aad), 3-aminoadipic acid (βAad), 2-aminosuberic acid (Asu)(acid), 2-aminopimelic acid (Apm)
<lysine>homolysine (hLys), ornithine (Orn), 2,4-diaminobutyric acid (2,4-A2bu), 2,3-diaminobutyric acid (2,3-A₂bu), 2,3-diaminopropanoic acid (A2pr)
<arginine>homoarginine (hArg), nor-arginine (nArg)
<histidine>homohistidine (hHis)
   (5) Amino acids having a substituted side chain

Examples of the amino acid having a substituted side chain are indicated below along with the corresponding natural proteinogenic amino acids.
<glycine>t-butylglycine (tbGly), 2-propargylglycine (pGly), cyclohexylglycine (chGly), cyclopentylglycine (cpGly), allylglycine (aGly)
<alanine>t-butylalanine (tbAla), cyclohexylalanine (chAla), cyclobutylalanine (cbAla), cyclopropylalanine (cpAla), 1-naphthyl alanine (1npAla), 2-naphthyl alanine (2npAla), benzothienylalanine (btAla), thienylalanine (thAla), thiazolylalanine (taAla), furylalanine (fAla), pyridine-2-ylalanine (2pyAla), pyridine-3-ylalanine (3pAla), pyridine-4-ylalanine (4pyAla), 1-piperazinyl alanine (ppAla), styrylalanine (stAla), anthryl alanine (anAla)
<methionine>selenomethionine (Sem), S-methylmethionine (mMet), methionine sulfoxide (Meo), methionine sulfone (Moo)
<proline>3-hydroxyproline (3Hyp), 4-hydroxyproline (4Hyp), 5-hydroxyproline (5Hyp), 4-mercaptoproline (HSPro)
<phenylalanine>2-methylphenylalanine (OmPhe), 3-methylphenylalanine (MmPhe), 4-methylphenylalanine (PmPhe), 2-nitrophenylalanine (OnPhe), 3-nitrophenylalanine (MnPhe), 4-nitrophenylalanine (PnPhe), 2-cyanophenylalanine (OcnPhe), 3-cyanophenylalanine (McnPhe), 4-cyanophenylalanine (PcnPhe), 3,3-diphenylalanine (ffAla), 2-trifluoromethylphenylalanine (OtPhe), 3-trifluoromethylphenylalanine (MtPhe), 4-trifluoromethylphenylalanine (PtPhe), 4-aminophenylalanine (PaPhe), 4-aminomethylphenylalanine (PamPhe), 3,4-dimethoxyphenylalanine (PmmDopa), 3,4-dihydroxyphenylalanine (Dopa), phosphonomethylphenylalanine (phmPhe), phenylalanine in which the aromatic ring has been substituted by a halogen (e.g., 2-chlorophenylalanine (OcPhe), 3-chlorophenylalanine (McPhe), 4-chlorophenylalanine (PcPhe), 2-fluorophenylalanine (OfPhe), 3-fluorophenylalanine (MfPhe), 4-fluorophenylalanine (PfPhe), 2-bromophenylalanine (ObPhe), 3-bromophenylalanine (MbPhe), 4-bromophenylalanine (PbPhe), 2-iodophenylalanine (OiPhe), 3-iodophenylalanine (MiPhe), 4-iodophenylalanine (PiPhe), 2,4-dichlorophenylalanine (OpcPhe), 3,4-dichlorophenylalanine (MpcPhe), 2,4-difluorophenylalanine (OpfPhe), 3,4-difluorophenylalanine (MpfPhe), 3,4,5-trifluorylphenylalanine (PmmfPhe), pentafluorophenylalanine (5fPhe))
<tryptophan>5-fluorotryptophan (5fTyp), 6-fluorotryptophan (6fTyp), 1-methyltryptophan (1mTyp)
<tyrosine>monoiodotyrosine (iTyr), diiodotyrosine (iiTyr), triiodothyronine (iiiThy), O-methyltyrosine (mTyr), 3-nitrotyrosine (nTyr), phosphotyrosine (pTyr), 3-amino-tyrosine (MaTyr)
<cysteine>cysteineacid (Cya), S-benzyl-L-cysteine (bzCys), selenocysteine (Sec), penicillamine (Pen)
<serine>O-methylserine (mSer), phosphoserine (pSer)
<threonine>phosphothreonine (pThr),
<glutamic acid>γ-hydroxyglutamic acid (hoGlu), γ-carboxyglutamic acid (caGlu)
<lysine>Ne-acetyl lysine (AcLys), homocitrulline (Hci), 2,6-diaminopimelic acid (A2 pm), allohydroxylysine (AHyl), 2,6-diamino-4-hexyneacid (DaHea)
<arginine>ω-nitroarginine (ntArg), ω-amino-arginine (Oar), ω-methyl-arginine (Omr), citrulline (Cit)
<histidine>1-methylhistidine (lmHis), 3-methylhistidine (3mHis), 5-hydroxylysine (50HLys)

### (6) Other Non-Proteinogenic Amino Acids

For example, 3-amino-5-phenylpentanoic acid (Afp), 1,2,3,4-tetrahydroisoquinoline-3-carboxylic acid (Tic), ω-hydroxy-nor-arginine (HOnArg), 1-aminocyclopentane-3-carboxylic acid (Acp3Ca), thiazolidine-4-carboxylic acid (thz) and 5,5-dimethyl-D-thiazolidine-4-carboxylic acid (dtc) can be mentioned but not limited to.

### (7) Amino Acids Useful for Cyclization

### i) Amino Acids Having Chloroacetyl Group

Any amino acids in which amino group in the main chain or side chain is substituted with chloroacetyl group or 2-chloroacetamido benzoyl group (for example, the amino acids listed in Table 1)

Examples: N-chloroacetyl alanine, N-chloroacetylphenylalanine, N-chloroacetyl tyrosine, N-chloroacetyl tryptophan, N-3-(2-chloroacetamido)benzoylphenylalanine, N-3-(2-chloroacetamido)benzoyltyrosine, N-3-(2-chloroacetamido) benzoyltryptophan, β-N-chloroacetyl diaminopropanoic acid, γ-N-chloroacetyl diaminobutyric acid, σ-N-chloroacetyl ornithine, ε-N-chloroacetyl lysine

### ii) Amino Acids Having Ethynyl Group

PropargyIglycine, homopropargylglycine, 2-amino-6-heptynoic acid, 2-amino-7-octynic acid, 2-amino-8-nonynoic acid

Any amino acids in which an amino group in the main chain or the side chain is substituted with 4-pentinoyl group, 5-hexynoyl group, 4-pentinoylamidobenzoyl group or 5-hexynoylamidobenzoyl group (e.g., amino acids listed in Table 1) Examples: N-(4-pentinoyl)alanine, N-(4-pentinoyl)phenylalanine, N-(4-pentinoyl)tyrosine, N-(4-pentinoyl)tryptophan, N-3-(4-pentinoylamido)benzoylphenylalanine, N-3-(4-pentinoylamido)benzoyltyrosine, N-3-(4-pentinoylamido)benzoyltryptophan, β-N-(4-pentinoyl)diaminopropanoic acid, γ-N-(4-pentinoyl)diaminobutyric acid, σ-N-(4-pentinoyl)ornithine, ε-N-(4-pentinoyl)lysine, N-(5-hexynoyl)alanine, N-(5-hexynoyl)phenylalanine, N-(5-hexynoyl)tyrosine, N-(5-hexynoyl)tryptophan, N-3-(5-hexynoylamido)benzoylphenylalanine, N-3-(5-hexynoylamido)benzoyltyrosine, N-3-(5-hexynoylamido)benzoyltryptophan, β-N-(5-hexynoyl)diaminopropanoic acid, γ-N-(5-hexynoyl)diaminobutyric acid, σ-N-(5-hexynoyl)ornithine, ε-N-(5-hexynoyl)lysine

iii) Amino Acids Having Azido Group

Azidealanine, 2-amino-4-azidebutanoic acid, 5-azidenorvaline, azidenorleucine, 2-amino-7-azideheptane acid and 2-amino-8-azideoctaneacid

Any amino acids in which an amino group in the main chain or the side chain is substituted with azideacetyl group, 4-pentinoylamidobenzoyl group or 3-azidepentanoyl group (e.g., amino acids listed in Table 1) Examples: N-azideacetyl alanine, N-azideacetylphenylalanine, N-azideacetyl tyrosine, N-azideacetyl tryptophan, N-3-(4-pentinoylamido)benzoylphenylalanine, N-3-(4-pentinoylamido)benzoyltyrosine, N-3-(4-pentinoylamido)benzoyltryptophan, β-N-azideacetyl diaminopropanoic acid, γ-N-azideacetyl diaminobutyric acid, σ-N-azideacetyl ornithine, ε-N-azideacetyl lysine, N-3-azidepentanoylalanine, N-3-azidepentanoylphenylalanine, N-3-azidepentanoyltyrosine, N-3-azidepentanoyltryptophan, β-N-3-azidepentanoyldiaminopropanoic acid, γ-N-3-azidepentanoyldiaminobutyric acid, σ-N-3-azidepentanoylornithine, ε-N-3-azidepentanoyllysine

### iv) Amino Acids Having Benzylamino Group

Amino acids amino having a side chain containing an aromatic ring, in which the aromatic ring in the side chain is substituted with aminomethyl group (Table 1) Examples: N-(4-aminomethylbenzoyl)phenylalanine (AMBF), 3-aminomethyltyrosine

### v) Amino Acids Having 5-Hydroxyindole

Examples: 5-hydroxytryptophan

vi) Amino acids having —C≡C—CH₂—X, —C═C—CH₂—X or —Ar—CH₂—X, wherein Ar is an aromatic ring which optionally has a substituent, X is a leaving group, and examples of the leaving group include a halogen atom including Cl, Br and I, or the like Examples: 2-amino-6-chlorohexyne acid, 2-amino-7-chloroheptynoic acid, 2-amino-8-chlorooctynic acid, N-3-chloromethylbenzoylphenylalanine, N-3-chloromethylbenzoyltyrosine, N-3-chloromethylbenzoyltryptophan

Amino acids can be classified based on the property of their side chain. For example, amino acids can be classified into families including “hydrophobic amino acid” and “hydrophilic amino acid” based on the hydrophobicity of their side chain. Among hydrophobic amino acids, those having an alkyl group in their side chain refer to “aliphatic hydrophobic amino acids” and those having an aromatic ring in their side chain refer to “aromatic hydrophobic amino acids”. Among aliphatic hydrophobic amino acids, those having a branched alkyl group in the side chain refer to branched aliphatic hydrophobic amino acids. Hydrophilic amino acids can be classified into families including “neutral hydrophilic amino acids” and “basic amino acids” based on the charge of their side chain.

In addition, amino acids can be classified based on the kind of a substituent or an atom contained in their side chain. For example, they can be classified into “amino acids having a side chain containing aromatic ring”, “amino acids having a side chain containing amino group”, “amino acids having a side chain containing carboxyl group”, “amino acids having a side chain containing hydroxyl group”, “amino acids having a side chain containing thiol group”, “amino acids having a side chain containing amido group”, “amino acids having a side chain containing imino group”, “amino acids having a side chain containing a heterocycle”, “amino acids having a side chain containing sulfur atom” and the like. One amino acid may belong to plural categories. Many of neutral hydrophilic amino acids may be classified as a family of “amino acids having a side chain containing hydroxyl, group”, “amino acids having a side chain containing thiol group” or “amino acids having a side chain containing amido group”. Many of basic amino acids may be classified as “amino acids having a side chain containing amino group”. Many of acidic amino acids may be classified as “amino acids having a side chain containing carboxyl group”.

Examples of the amino acid classification are shown in Table I. Codes of family of amino acids in Table 1 are as follows.
hydrophobic: hydrophobic amino acid
aliphatic: aliphatic hydrophobic amino acid
branched: branched aliphatic hydrophobic amino acid
aromatic: aromatic hydrophobic amino acid
hydrophilic: hydrophilic amino acid
basic: basic amino acid
acidic: acidic amino acid
neutral: neutral hydrophilic amino acid
aromatic ring: amino acid having a side chain containing aromatic ring
amino: amino acid having a side chain containing amino group
carboxy: amino acid having a side chain containing carboxy group
hydroxy: amino acid having a side chain containing hydroxyl group
thiol: amino acid having a side chain containing thiol group
amido: amino acid having a side chain containing amido group
imino: amino acid having a side chain containing imino group
heterocycle: amino acid having a side chain containing a heterocycle
sulfur: an amino acid having a side chain containing sulfur atom

TABLE 1

| | | Abbreviation | | | | | | | | |
|---|---|---|---|---|---|---|---|---|---|---|
| | Abbreviation | (three or | Crassification according to hydrophobicity of side chain | | | | | | | |
| Name | (one-letter) | more letters) | hydrophobic | aliphatic | branched | aromatic | hydrophilic | basic | acidic | neutral |
| glycine | G | Gly | ◯ | ◯ | | | | | | |
| alanine | A | Ala | ◯ | ◯ | | | | | | |
| leucine | L | Leu | ◯ | ◯ | ◯ | | | | | |
| isoleucine | I | Ile | ◯ | ◯ | ◯ | | | | | |
| valine | V | Val | ◯ | ◯ | ◯ | | | | | |
| methionine | M | Met | ◯ | | | | | | | |
| proline | P | Pro | ◯ | | | | | | | |
| phenylalanine | F | Phe | ◯ | | | ◯ | | | | |
| tryptophan | W | Typ | ◯ | | | ◯ | | | | |
| tyrosine | Y | Tyr | | | | | ◯ | | | ◯ |
| cysteine | C | Cys | | | | | ◯ | | | ◯ |
| serine | S | Ser | | | | | ◯ | | | ◯ |
| threonine | T | Thr | | | | | ◯ | | | ◯ |
| asparagine | N | Asn | | | | | ◯ | | | ◯ |
| glutamine | Q | Gln | | | | | ◯ | | | ◯ |
| aspartic acid | D | Asp | | | | | ◯ | | ◯ | |
| glutamic acid | E | Glu | | | | | ◯ | | ◯ | |
| lysine | K | Lys | | | | | ◯ | ◯ | | |
| arginine | R | Arg | | | | | ◯ | ◯ | | |
| histidine | H | His | | | | | ◯ | ◯ | | |
| D-alanine | s | ala | ◯ | ◯ | | | | | | |
| D-leucine | l | leu | ◯ | ◯ | ◯ | | | | | |
| D-isoleucine | i | ile | ◯ | ◯ | ◯ | | | | | |
| D-valine | v | val | ◯ | ◯ | ◯ | | | | | |
| D-methionine | m | met | ◯ | | | | | | | |
| D-proline | p | pro | ◯ | | | | | | | |
| D-phenylalanine | f | phe | ◯ | | | ◯ | | | | |
| D-tryptophan | w | typ | ◯ | | | ◯ | | | | |
| D-tyrosine | y | tyr | | | | | ◯ | | | ◯ |
| D-cysteine | c | cys | | | | | ◯ | | | ◯ |
| D-serine | s | ser | | | | | ◯ | | | ◯ |
| D-threonine | t | thr | | | | | ◯ | | | ◯ |
| D-asparagine | n | asn | | | | | ◯ | | | ◯ |
| D-glutamine | q | gln | | | | | ◯ | | | ◯ |
| D-aspartic acid | d | asp | | | | | ◯ | | ◯ | |
| D-glutamic acid | e | glu | | | | | ◯ | | ◯ | |
| D-lysine | k | lys | | | | | ◯ | ◯ | | |
| D-arginine | r | arg | | | | | ◯ | ◯ | | |
| D-histidine | h | his | | | | | ◯ | ◯ | | |
| alloisoleucine | | AIle | ◯ | ◯ | ◯ | | | | | |
| allothreonine | | alThr | | | | | ◯ | | | ◯ |
| β-alanine | | bAla | ◯ | ◯ | | | | | | |
| β-leucine | | bLeu | ◯ | ◯ | ◯ | | | | | |
| β-methionine | | bMet | ◯ | | | | | | | |
| β-phenylalanine | | bPhe | ◯ | | | ◯ | | | | |
| β-tyrosine | | bTyr | | | | | ◯ | | | ◯ |
| β-cysteine | | bCys | | | | | ◯ | | | ◯ |
| β-serine | | bSer | | | | | ◯ | | | ◯ |
| β-threonine | | bThr | | | | | ◯ | | | ◯ |
| β-asparagine | | bAsn | | | | | ◯ | | | ◯ |
| β-glutamine | | bGln | | | | | ◯ | | | ◯ |
| β-aspartic acid | | bAsp | | | | | ◯ | | ◯ | |
| β-glutamic acid | | bGlu | | | | | ◯ | | ◯ | |
| β-lysine | | bLys | | | | | ◯ | ◯ | | |
| β-arginine | | bArg | | | | | ◯ | ◯ | | |
| β-histidine | | bHis | | | | | ◯ | ◯ | | |
| isoaspartic acid | | iAsp | | | | | ◯ | | ◯ | |
| isoglutamic acid | | iGlu | | | | | ◯ | | ◯ | |
| norleucine | | Nle | ◯ | ◯ | | | | | | |
| tert-leucine | | Tle | ◯ | ◯ | ◯ | | | | | |
| norvaline | | Nva | ◯ | ◯ | | | | | | |
| β-homoalanine | | Bha | ◯ | ◯ | | | | | | |
| β-homovaline | | Bhv | ◯ | ◯ | ◯ | | | | | |
| β-homoleucine | | Bhi | ◯ | ◯ | ◯ | | | | | |
| β-homoisoleucine | | Bhi | ◯ | ◯ | ◯ | | | | | |
| β-homomethionine | | Bhm | ◯ | | | | | | | |
| β-homophenylalanine | | Bhf | ◯ | | | ◯ | | | | |
| β-homotryptophane | | Bhw | ◯ | | | ◯ | | | | |
| β-homotyrosine | | Bhy | | | | | ◯ | | | ◯ |
| β-homocysteine | | Bhc | | | | | ◯ | | | ◯ |
| β-homoserine | | Bhs | | | | | ◯ | | | ◯ |
| β-homothreonine | | Bht | | | | | ◯ | | | ◯ |
| β-homoasparagine | | Bhn | | | | | ◯ | | | ◯ |
| β-homoglutamine | | Bhq | | | | | ◯ | | | ◯ |
| β-homoaspartic acid | | Bhe | | | | | ◯ | | ◯ | |
| β-homoglutamine acid | | Bhd | | | | | ◯ | | ◯ | |
| β-homolysine | | Bhk | | | | | ◯ | ◯ | | |
| β-homoarginine | | Bhr | | | | | ◯ | ◯ | | |
| β-homohistidine | | Bhh | | | | | ◯ | ◯ | | |
| 3-aminobutyric acid | | 3Abu | ◯ | ◯ | | | | | | |
| 4-aminobutyric acid | | 4Abu | ◯ | ◯ | | | | | | |
| 6-aminohexanoic acid | | ε Ahx | ◯ | ◯ | | | | | | |
| 5-aminopentanoic acid | | δ Ava | ◯ | ◯ | | | | | | |
| N-methylglycine | | MeGly | ◯ | ◯ | | | | | | |
| (sarcosine) | | | | | | | | | | |
| N-methylalanine | | MeAla | ◯ | ◯ | | | | | | |
| N-methylleucine | | MeLeu | ◯ | ◯ | ◯ | | | | | |
| N-methylisoleucine | | MeIle | ◯ | ◯ | ◯ | | | | | |
| N-methylvaline | | MeVal | ◯ | ◯ | ◯ | | | | | |
| N-methylmethionine | | MeMet | ◯ | | | | | | | |
| N-methylproline | | MePro | ◯ | | | | | | | |
| N-methylphenylalanine | | MePhe | ◯ | | | ◯ | | | | |
| N-methyltryptophan | | MeTyp | ◯ | | | ◯ | | | | |
| N-methyltyrosine | | MeTyr | | | | | ◯ | | | ◯ |
| N-methylcysteine | | MeCys | | | | | ◯ | | | ◯ |
| N-methylserine | | MeSer | | | | | ◯ | | | ◯ |
| N-methylthreonine | | MeThr | | | | | ◯ | | | ◯ |
| N-methylasparagine | | MeAsn | | | | | ◯ | | | ◯ |
| N-methylglutamine | | MeGln | | | | | ◯ | | | ◯ |
| N-methylaspartic acid | | MeAsp | | | | | ◯ | | ◯ | |
| N-methylglutamic acid | | MeGlu | | | | | ◯ | | ◯ | |
| N-methyllysine | | MeLys | | | | | ◯ | ◯ | | |
| N-methylarginine | | MeArg | | | | | ◯ | ◯ | | |
| N-methylhistidine | | MeHis | | | | | ◯ | ◯ | | |
| N-ethylglycine | | EtGly | ◯ | ◯ | | | | | | |
| N-propylglycine | | PrGly | ◯ | ◯ | | | | | | |
| N-(4- | | AbGly | ◯ | ◯ | | | | | | |
| aminobutyl)glycine | | | | | | | | | | |
| N-(4- | | ApGly | ◯ | ◯ | | | | | | |
| aminopropyl)glycine | | | | | | | | | | |
| N-ethylasparagine | | EtAsn | | | | | ◯ | | | ◯ |
| N-formylmethionine | | FoMet | ◯ | | | | | | | |
| N-mercaptoethylglycine | | NHSEtGly | ◯ | | | | | | | |
| α-aminoisobutyric | | Aib | | ◯ | | | | | | |
| acid (α-methylalanine) | | | | | | | | | | |
| α-methylleucine | | Aml | ◯ | ◯ | ◯ | | | | | |
| α-methylproline | | Amp | ◯ | | | | | | | |
| α,α-diethylglycine | | Deg | ◯ | ◯ | | | | | | |
| α,α-dibutylglycine | | Dbg | ◯ | ◯ | | | | | | |
| 1-amino-cyclohexane- | | Ac6c | ◯ | ◯ | | | | | | |
| 1-carboxylic acid | | | | | | | | | | |
| 1-amino- | | Ac5c | ◯ | ◯ | | | | | | |
| cyclopentane-1- | | | | | | | | | | |
| carboxylic acid | | | | | | | | | | |
| 1-amino- | | Ac3c | ◯ | ◯ | | | | | | |
| cyclopropane-1- | | | | | | | | | | |
| carboxylic acid | | | | | | | | | | |
| 1-amino-(4-N- | | Apc | ◯ | ◯ | | | | | | |
| piperidinyl) carboxylic | | | | | | | | | | |
| acid | | | | | | | | | | |
| homoalanine | | hAla | ◯ | ◯ | | | | | | |
| 2-aminopentanoic acid | | 2Apn | ◯ | ◯ | | | | | | |
| homoleucine | | hLeu | ◯ | ◯ | ◯ | | | | | |
| homoisoleucine | | hIle | ◯ | ◯ | ◯ | | | | | |
| homomethionine | | hMet | ◯ | | | | | | | |
| pipecolic acid | | Pip | ◯ | | | | | | | |
| azetidine-3-carboxylic | | Az3Ca | ◯ | | | | | | | |
| acid | | | | | | | | | | |
| azetidine-2-carboxylic | | Az2Ca | ◯ | | | | | | | |
| lacid | | | | | | | | | | |
| homophenylalanine | | hPhe | ◯ | | | ◯ | | | | |
| phenylglycine | | fGly | ◯ | | | ◯ | | | | |
| homotryptophane | | hTyp | ◯ | | | ◯ | | | | |
| homotyrosine | | hTyr | | | | | ◯ | | | ◯ |
| homocysteine | | hCys | | | | | ◯ | | | ◯ |
| horcysteine | | Ncy | | | | | ◯ | | | ◯ |
| homoserine | | hSer | | | | | ◯ | | | ◯ |
| homothreonine | | hThr | | | | | ◯ | | | ◯ |
| homoglutamine | | hGln | | | | | ◯ | | | ◯ |
| homoglutamine acid | | hGlu | | | | | ◯ | | ◯ | |
| 2-aminoadipic acid | | Aad | | | | | ◯ | | ◯ | |
| 3-aminoadipic acid | | β Aad | | | | | ◯ | | ◯ | |
| 2-aminosuberic acid | | Asu | | | | | ◯ | | ◯ | |
| 2-aminopimeric acid | | Apm | | | | | ◯ | | ◯ | |
| homolysine | | hLys | | | | | ◯ | ◯ | | |
| ornithine | | Orn | | | | | ◯ | ◯ | | |
| 2,4-diaminobutyric | | 2,4-A2bu | | | | | ◯ | ◯ | | |
| acid | | | | | | | | | | |
| 2,3-diaminobutyric | | 2,3-A2bu | | | | | ◯ | ◯ | | |
| acid | | | | | | | | | | |
| 2,3-diaminopropanoic | | A2pr | | | | | ◯ | ◯ | | |
| acid | | | | | | | | | | |
| homoarginine | | hArg | | | | | ◯ | ◯ | | |
| nor-arginine | | nArg | | | | | ◯ | ◯ | | |
| homohistidine | | hHis | | | | | ◯ | ◯ | | |
| t-butylglycine | | tbGly | ◯ | ◯ | ◯ | | | | | |
| 2-propargylglycine | | pGly | ◯ | ◯ | | | | | | |
| cyclohexylglycine | | chGly | ◯ | ◯ | ◯ | | | | | |
| cyclopentylglycine | | aGly | ◯ | ◯ | | | | | | |
| t-butylalanine | | tbAla | ◯ | ◯ | ◯ | | | | | |
| cyclohexylalanine | | chAla | ◯ | ◯ | ◯ | | | | | |
| cyclobutylalanine | | cbAla | ◯ | ◯ | ◯ | | | | | |
| cyclopropylalanine | | cpAla | ◯ | ◯ | ◯ | | | | | |
| 1-naphthyl alanine | | 1npAla | ◯ | | | ◯ | | | | |
| 2-naphthyl alanine | | 2npAla | ◯ | | | ◯ | | | | |
| benzothienylalanine | | btAla | ◯ | | | ◯ | | | | |
| thienylalanine | | thAla | ◯ | | | ◯ | | | | |
| thiazolylalanine | | taAla | ◯ | | | ◯ | | | | |
| furylalanine | | FAla | ◯ | | | ◯ | | | | |
| pyridine-2-ylalanine | | 2pyAla | | | | | ◯ | ◯ | | |
| pyridine-3-ylalanine | | 3pyAla | | | | | ◯ | ◯ | | |
| pyridine-4-ylalanine | | 4pyAla | | | | | ◯ | ◯ | | |
| 1-piperazinyl alanine | | ppAla | | | | | ◯ | ◯ | | |
| styrylalanine | | stAla | ◯ | | | ◯ | | | | |
| anthryl alanine | | anAla | ◯ | | | ◯ | | | | |
| selenomethionine | | Sem | ◯ | | | | | | | |
| S-methylmethionine | | mMet | ◯ | | | | | | | |
| methionine sulfoxide | | Meo | | | | | ◯ | | | ◯ |
| methionine sulfone | | Moo | | | | | ◯ | | ◯ | |
| 3-hydroxyproline | | 3Hyp | | | | | ◯ | | | ◯ |
| 4-hydroxyproline | | 4Hyp | | | | | ◯ | | | ◯ |
| 5-hydroxyproline | | 5Hyp | | | | | ◯ | | | ◯ |
| 4-mercaptoproline | | HSPro | | | | | ◯ | | | ◯ |
| 2-methylphenylalanine | | OmPhe | ◯ | | | ◯ | | | | |
| 3-methylphenylalanine | | MmPhe | ◯ | | | ◯ | | | | |
| 4-methylphenylalanine | | PmPhe | ◯ | | | ◯ | | | | |
| 2-nitrophenylalanine | | OnPhe | ◯ | | | ◯ | | | | |
| 3-nitrophenylalanine | | MnPhe | ◯ | | | ◯ | | | | |
| 4-nitrophenylalanine | | PnPhe | ◯ | | | ◯ | | | | |
| 2-cyanophenylalanine | | OcnPha | | | | | ◯ | | | ◯ |
| 3-cyanophenylalanine | | McnPhe | | | | | ◯ | | | ◯ |
| 4-cyanophenylalanine | | PcnPhe | | | | | ◯ | | | ◯ |
| 3,3-diphenylalanine | | ffAla | ◯ | | | ◯ | | | | |
| 2- | | OtPhe | ◯ | | | ◯ | | | | |
| trifluoromethylphenylalanine | | | | | | | | | | |
| 3- | | MtPhe | ◯ | | | ◯ | | | | |
| trifluoromethylphenylalanine | | | | | | | | | | |
| 4- | | PtPhe | ◯ | | | ◯ | | | | |
| trifluoromethylphenylalanine | | | | | | | | | | |
| 4-aminophenylalanine | | PaPhe | | | | | ◯ | ◯ | | |
| 4- | | PamPhe | | | | | ◯ | ◯ | | |
| aminomethylphenylalanine | | | | | | | | | | |
| 3,4- | | PmmDopa | ◯ | | | ◯ | | | | |
| dimethoxyphenylalanine | | | | | | | | | | |
| 3,4- | | Dopa | | | | | ◯ | | | ◯ |
| dihydroxyphenylalanine | | | | | | | | | | |
| phosphonomethylphenylalanine | | phmPhe | | | | | ◯ | | ◯ | |
| 2-chlorophenylalanine | | OcPhe | ◯ | | | ◯ | | | | |
| 3-chlorophenylalanine | | McPhe | ◯ | | | ◯ | | | | |
| 4-chlorophenylalanine | | PcPhe | ◯ | | | ◯ | | | | |
| 2-bromophenylalanine | | ObPhe | ◯ | | | ◯ | | | | |
| 3-bromophenylalanine | | MbPhe | ◯ | | | ◯ | | | | |
| 4-bromophenylalanine | | PbPhe | ◯ | | | ◯ | | | | |
| 2-iodophenylalanine | | OiPhe | ◯ | | | ◯ | | | | |
| 3-iodophenylalanine | | MiPhe | ◯ | | | ◯ | | | | |
| 4-iodophenylalanine | | PiPhe | ◯ | | | ◯ | | | | |
| 2,4- | | OpcPhe | ◯ | | | ◯ | | | | |
| dichlorophenylalanine | | | | | | | | | | |
| 3,4- | | MpcPhe | ◯ | | | ◯ | | | | |
| dichlorophenylalanine | | | | | | | | | | |
| 2,4- | | OpfPhe | ◯ | | | ◯ | | | | |
| difluorophenylalanine | | | | | | | | | | |
| 3.4- | | MpfPhe | ◯ | | | ◯ | | | | |
| difluorophenylalanine | | | | | | | | | | |
| 3,4,5- | | PmmfPhe | ◯ | | | ◯ | | | | |
| trifluorophenylalanine | | | | | | | | | | |
| pentafluorophenylalanine | | 5fPhe | ◯ | | | ◯ | | | | |
| 5-fluorotryptophan | | 5fTyp | ◯ | | | ◯ | | | | |
| 6-fluorotryptophan | | 6fTyp | ◯ | | | ◯ | | | | |
| 1-methyltryptophan | | 1mTyp | ◯ | | | ◯ | | | | |
| monoiodotyrosine | | iTyr | | | | | ◯ | | | ◯ |
| dilodotyrosine | | iiTyr | | | | | ◯ | | | ◯ |
| triiodothyronine | | iiiThy | | | | | ◯ | | | ◯ |
| O-methyltyrosine | | mTyr | ◯ | | | ◯ | | | | |
| 3-nitrotyrosine | | nTyr | | | | | ◯ | | | ◯ |
| phosphotyrosine | | pTyr | | | | | ◯ | | ◯ | |
| 3-amino-tyrosine | | MaTyr | | | | | ◯ | ◯ | | |
| cysteineacid | | Cya | | | | | ◯ | | ◯ | |
| S-benzyl-L-cysteine | | bzCys | ◯ | | | ◯ | | | | |
| selenocysteine | | Sec | | | | | ◯ | | | ◯ |
| penicillamine | | Pen | | | | | ◯ | | | ◯ |
| 5-mercaptonorvaline | | shNva | | | | | ◯ | | | ◯ |
| 6-mercaptonorleucine | | shNle | | | | | ◯ | | | ◯ |
| 2-amino-7- | | shHep | | | | | ◯ | | | ◯ |
| mercaptoheptane acid | | | | | | | | | | |
| 2-amino-8- | | shOct | | | | | ◯ | | | ◯ |
| mercaptooctaneacid | | | | | | | | | | |
| O-methylserine | | mSer | ◯ | | | | | | | |
| phosphoserine | | pSer | | | | | ◯ | | ◯ | |
| phosphothreonine | | pThr | | | | | ◯ | | ◯ | |
| γ-hydroxyglutamic | | hoGlu | | | | | ◯ | | ◯ | |
| acid | | | | | | | | | | |
| γ-carboxyglutamio | | caGlu | | | | | ◯ | | ◯ | |
| acid | | | | | | | | | | |
| N ε-acetyl lysine | | AcLys | | | | | ◯ | | | ◯ |
| homocitrulline | | Hci | | | | | ◯ | | | ◯ |
| 2,6-diaminopimelic | | A2pm | | | | | ◯ | | | ◯ |
| acid | | | | | | | | | | |
| alohydroxyllysine | | Ahyl | | | | | ◯ | ◯ | | |
| 2,6-diamino-4- | | DaHea | | | | | ◯ | ◯ | | |
| hexyneacid | | | | | | | | | | |
| ω-nitroarginine | | ntArg | | | | | ◯ | ◯ | | |
| ω-amino-arginine | | Oar | | | | | ◯ | ◯ | | |
| ω-methyl-arginine | | Omr | | | | | ◯ | ◯ | | |
| citrulline | | Cit | | | | | ◯ | | | ◯ |
| 1-methylhistidine | | 1mHis | | | | | ◯ | ◯ | | |
| 3-methylhistidine | | 3mHis | | | | | ◯ | ◯ | | |
| 5-methylhistidine | | 5mHis | | | | | ◯ | ◯ | | |
| 3-amino-5- | | Afp | ◯ | | | ◯ | | | | |
| phenylpentanoic acid | | | | | | | | | | |
| 1,2,3,4- | | Tic | ◯ | | | ◯ | | | | |
| tetrahydroisoquinoline- | | | | | | | | | | |
| 3-carboxylic acid | | | | | | | | | | |
| ω-hydroxy-nor- | | HOnArg | | | | | ◯ | ◯ | | |
| arginine | | | | | | | | | | |
| 1-aminocyclopentane- | | Acp3Ca | ◯ | | | | | | | |
| 3-carboxylic acid | | | | | | | | | | |
| thiazolidine-4- | | thz | ◯ | | | | | | | |
| carboxylic acid | | | | | | | | | | |
| 5,5-dimethyl-D- | | dtc | ◯ | | | | | | | |
| thiazolidine-4- | | | | | | | | | | |
| carboxylic acid | | | | | | | | | | |

| | Crassification according to kind of substituent or the like contained in side chain | | | | | | | | |
|---|---|---|---|---|---|---|---|---|---|
| Name | aromatic ring | amino | carboxy | hydroxy | thiol | amido | imina | heterocycle | sulfur |
| glycine | | | | | | | | | |
| alanine | | | | | | | | | |
| leucine | | | | | | | | | |
| isoleucine | | | | | | | | | |
| valine | | | | | | | | | |
| methionine | | | | | | | | | ◯ |
| proline | | | | | | | ◯ | ◯ | |
| phenylalanine | ◯ | | | | | | | | |
| tryptophan | ◯ | | | | | | | ◯ | |
| tyrosine | ◯ | | | ◯ | | | | | |
| cysteine | | | | | ◯ | | | | ◯ |
| serine | | | | ◯ | | | | | |
| threonine | | | | ◯ | | | | | |
| asparagine | | | | | | ◯ | | | |
| glutamine | | | | | | ◯ | | | |
| aspartic acid | | | ◯ | | | | | | |
| glutamic acid | | | ◯ | | | | | | |
| lysine | | ◯ | | | | | | | |
| arginine | | ◯ | | | | | | | |
| histidine | | ◯ | | | | | | ◯ | |
| D-alanine | | | | | | | | | |
| D-leucine | | | | | | | | | |
| D-isoleucine | | | | | | | | | |
| D-valine | | | | | | | | | |
| D-methionine | | | | | | | | | ◯ |
| D-proline | | | | | | | ◯ | ◯ | |
| D-phenylalanine | ◯ | | | | | | | | |
| D-tryptophan | ◯ | | | | | | | ◯ | |
| D-tyrosine | ◯ | | | ◯ | | | | | |
| D-cysteine | | | | | ◯ | | | | ◯ |
| D-serine | | | | ◯ | | | | | |
| D-threonine | | | | ◯ | | | | | |
| D-asparagine | | | | | | ◯ | | | |
| D-glutamine | | | | | | ◯ | | | |
| D-aspartic acid | | | ◯ | | | | | | |
| D-glutamic acid | | | ◯ | | | | | | |
| D-lysine | | ◯ | | | | | | | |
| D-arginine | | ◯ | | | | | | | |
| D-histidine | | ◯ | | | | | | ◯ | |
| alloisoleucine | | | | | | | | | |
| allothreonine | | | | ◯ | | | | | |
| β-alanine | | | | | | | | | |
| β-leucine | | | | | | | | | |
| β-methionine | | | | | | | | | ◯ |
| β-phenylalanine | ◯ | | | | | | | | |
| β-tyrosine | ◯ | | | | | | | | |
| β-cysteine | | | | | ◯ | | ◯ | | ◯ |
| β-serine | | | | ◯ | | | | | |
| β-threonine | | | | ◯ | | | | | |
| β-asparagine | | | | | | ◯ | | | |
| β-glutamine | | | | | | ◯ | | | |
| β-aspartic acid | | | ◯ | | | | | | |
| β-glutamic acid | | | ◯ | | | | | | |
| β-lysine | | ◯ | | | | | | | |
| β-arginine | | ◯ | | | | | | | |
| β-histidine | | ◯ | | | | | | ◯ | |
| isoaspartic acid | | | ◯ | | | | | | |
| isoglutamic acid | | | ◯ | | | | | | |
| norleucine | | | | | | | | | |
| tert-leucine | | | | | | | | | |
| norvaline | | | | | | | | | |
| β-homoalanine | | | | | | | | | |
| β-homovaline | | | | | | | | | |
| β-homoleucine | | | | | | | | | |
| β-homoisoleucine | | | | | | | | | |
| β-homomethionine | | | | | | | | | ◯ |
| β-homophenylalanine | ◯ | | | | | | | | |
| β-homotryptophane | ◯ | | | | | | | ◯ | |
| β-homotyrosine | ◯ | | | | | | | | |
| β-homocysteine | | | | | ◯ | | ◯ | | ◯ |
| β-homoserine | | | | ◯ | | | | | |
| β-homothreonine | | | | ◯ | | | | | |
| β-homoasparagine | | | | | | ◯ | | | |
| β-homoglutamine | | | | | | ◯ | | | |
| β-homoaspartic acid | | | ◯ | | | | | | |
| β-homoglutamine acid | | | ◯ | | | | | | |
| β-homolysine | | ◯ | | | | | | | |
| β-homoarginine | | ◯ | | | | | | | |
| β-homohistidine | | ◯ | | | | | | ◯ | |
| 3-aminobutyric acid | | | | | | | | | |
| 4-aminobutyric acid | | | | | | | | | |
| 6-aminohexanoic acid | | | | | | | | | |
| 5-aminopentanoic acid | | | | | | | | | |
| N-methylglycine | | | | | | | | | |
| (sarcosine) | | | | | | | | | |
| N-methylalanine | | | | | | | | | |
| N-methylleucine | | | | | | | | | |
| N-methylisoleucine | | | | | | | | | |
| N-methylvaline | | | | | | | | | |
| N-methylmethionine | | | | | | | | | ◯ |
| N-methylproline | | | | | | | ◯ | ◯ | |
| N-methylphenylalanine | ◯ | | | | | | | | |
| N-methyltryptophan | ◯ | | | | | | | ◯ | |
| N-methyltyrosine | ◯ | | | | | | | | |
| N-methylcysteine | | | | | ◯ | | ◯ | | ◯ |
| N-methylserine | | | | ◯ | | | | | |
| N-methylthreonine | | | | ◯ | | | | | |
| N-methylasparagine | | | | | | ◯ | | | |
| N-methylglutamine | | | | | | ◯ | | | |
| N-methylaspartic acid | | | ◯ | | | | | | |
| N-methylglutamic acid | | | ◯ | | | | | | |
| N-methyllysine | | ◯ | | | | | | | |
| N-methylarginine | | ◯ | | | | | | | |
| N-methylhistidine | | ◯ | | | | | | ◯ | |
| N-ethylglycine | | | | | | | | | |
| N-propylglycine | | | | | | | | | |
| N-(4- | | | | | | | | | |
| aminobutyl)glycine | | | | | | | | | |
| N-(4- | | | | | | | | | |
| aminopropyl)glycine | | | | | | | | | |
| N-ethylasparagine | | | | | | ◯ | | | |
| N-formylmethionine | | | | | | | | | ◯ |
| N-mercaptoethylglycine | | | | | ◯ | | | | |
| α-aminoisobutyric | | | | | | | | | |
| acid (α-methylalanine) | | | | | | | | | |
| α-methylleucine | | | | | | | | | |
| α-methylproline | | | | | | | ◯ | ◯ | |
| α,α-diethylglycine | | | | | | | | | |
| α,α-dibutylglycine | | | | | | | | | |
| 1-amino-cyclohexane- | | | | | | | | | |
| 1-carboxylic acid | | | | | | | | | |
| 1-amino- | | | | | | | | | |
| cyclopentane-1- | | | | | | | | | |
| carboxylic acid | | | | | | | | | |
| 1-amino- | | | | | | | | | |
| cyclopropane-1- | | | | | | | | | |
| carboxylic acid | | | | | | | | | |
| 1-amino-(4-N- | | ◯ | | | | | | ◯ | |
| piperidinyl) carboxylic | | | | | | | | | |
| acid | | | | | | | | | |
| homoalanine | | | | | | | | | |
| 2-aminopentanoic acid | | | | | | | | | |
| homoleucine | | | | | | | | | |
| homoisoleucine | | | | | | | | | |
| homomethionine | | | | | | | | | ◯ |
| pipecolic acid | | | | | | | ◯ | ◯ | |
| azetidine-3-carboxylic | | | | | | | ◯ | ◯ | |
| acid | | | | | | | | | |
| azetidine-2-carboxylic | | | | | | | ◯ | ◯ | |
| lacid | | | | | | | | | |
| homophenylalanine | ◯ | | | | | | | | |
| phenylglycine | ◯ | | | | | | | | |
| homotryptophane | ◯ | | | | | | | ◯ | |
| homotyrosine | ◯ | | | | | | | | |
| homocysteine | | | | | ◯ | | ◯ | | ◯ |
| horcysteine | | | | | ◯ | | | | |
| homoserine | | | | ◯ | | | | | |
| homothreonine | | | | ◯ | | | | | |
| homoglutamine | | | | | | ◯ | | | |
| homoglutamine acid | | | ◯ | | | | | | |
| 2-aminoadipic acid | | | ◯ | | | | | | |
| 3-aminoadipic acid | | | ◯ | | | | | | |
| 2-aminosuberic acid | | | ◯ | | | | | | |
| 2-aminopimeric acid | | | ◯ | | | | | | |
| homolysine | | ◯ | | | | | | | |
| ornithine | | ◯ | | | | | | | |
| 2,4-diaminobutyric | | ◯ | | | | | | | |
| acid | | | | | | | | | |
| 2,3-diaminobutyric | | ◯ | | | | | | | |
| acid | | | | | | | | | |
| 2,3-diaminopropanoic | | ◯ | | | | | | | |
| acid | | | | | | | | | |
| homoarginine | | ◯ | | | | | | | |
| nor-arginine | | ◯ | | | | | | | |
| homohistidine | | ◯ | | | | | | ◯ | |
| t-butylglycine | | | | | | | | | |
| 2-propargylglycine | | | | | | | | | |
| cyclohexylglycine | | | | | | | | | |
| cyclopentylglycine | | | | | | | | | |
| t-butylalanine | | | | | | | | | |
| cyclohexylalanine | | | | | | | | | |
| cyclobutylalanine | | | | | | | | | |
| cyclopropylalanine | | | | | | | | | |
| 1-naphthyl alanine | ◯ | | | | | | | | |
| 2-naphthyl alanine | ◯ | | | | | | | | |
| benzothienylalanine | ◯ | | | | | | | ◯ | ◯ |
| thienylalanine | ◯ | | | | | | | ◯ | ◯ |
| thiazolylalanine | ◯ | | | | | | | ◯ | ◯ |
| furylalanine | ◯ | | | | | | | ◯ | |
| pyridine-2-ylalanine | ◯ | | | | | | | ◯ | |
| pyridine-3-ylalanine | ◯ | | | | | | | ◯ | |
| pyridine-4-ylalanine | ◯ | | | | | | | ◯ | |
| 1-piperazinyl alanine | | ◯ | | | | | | ◯ | |
| styrylalanine | ◯ | | | | | | | | |
| anthryl alanine | ◯ | | | | | | | | |
| selenomethionine | | | | | | | | | |
| S-methylmethionine | | | | | | | | | ◯ |
| methionine sulfoxide | | | | | | | | | ◯ |
| methionine sulfone | | | | | | | | | ◯ |
| 3-hydroxyproline | | | | ◯ | | | ◯ | ◯ | |
| 4-hydroxyproline | | | | ◯ | | | ◯ | ◯ | |
| 5-hydroxyproline | | | | ◯ | | | ◯ | ◯ | |
| 4-mercaptoproline | | | | | ◯ | | ◯ | ◯ | |
| 2-methylphenylalanine | ◯ | | | | | | | | |
| 3-methylphenylalanine | ◯ | | | | | | | | |
| 4-methylphenylalanine | ◯ | | | | | | | | |
| 2-nitrophenylalanine | ◯ | | | | | | | | |
| 3-nitrophenylalanine | ◯ | | | | | | | | |
| 4-nitrophenylalanine | ◯ | | | | | | | | |
| 2-cyanophenylalanine | ◯ | | | | | | | | |
| 3-cyanophenylalanine | ◯ | | | | | | | | |
| 4-cyanophenylalanine | ◯ | | | | | | | | |
| 3,3-diphenylalanine | ◯ | | | | | | | | |
| 2- | ◯ | | | | | | | | |
| trifluoromethylphenylalanine | | | | | | | | | |
| 3- | ◯ | | | | | | | | |
| trifluoromethylphenylalanine | | | | | | | | | |
| 4- | ◯ | | | | | | | | |
| trifluoromethylphenylalanine | | | | | | | | | |
| 4-aminophenylalanine | ◯ | ◯ | | | | | | | |
| 4- | ◯ | ◯ | | | | | | | |
| aminomethylphenylalanine | | | | | | | | | |
| 3,4- | ◯ | | | | | | | | |
| dimethoxyphenylalanine | | | | | | | | | |
| 3,4- | ◯ | | | ◯ | | | | | |
| dihydroxyphenylalanine | | | | | | | | | |
| phosphonomethylphenylalanine | ◯ | | | | | | | | |
| 2-chlorophenylalanine | ◯ | | | | | | | | |
| 3-chlorophenylalanine | ◯ | | | | | | | | |
| 4-chlorophenylalanine | ◯ | | | | | | | | |
| 2-bromophenylalanine | ◯ | | | | | | | | |
| 3-bromophenylalanine | ◯ | | | | | | | | |
| 4-bromophenylalanine | ◯ | | | | | | | | |
| 2-iodophenylalanine | ◯ | | | | | | | | |
| 3-iodophenylalanine | ◯ | | | | | | | | |
| 4-iodophenylalanine | ◯ | | | | | | | | |
| 2,4- | ◯ | | | | | | | | |
| dichlorophenylalanine | | | | | | | | | |
| 3,4- | ◯ | | | | | | | | |
| dichlorophenylalanine | | | | | | | | | |
| 2,4- | ◯ | | | | | | | | |
| difluorophenylalanine | | | | | | | | | |
| 3.4- | ◯ | | | | | | | | |
| difluorophenylalanine | | | | | | | | | |
| 3,4,5- | ◯ | | | | | | | | |
| trifluorophenylalanine | | | | | | | | | |
| pentafluorophenylalanine | ◯ | | | | | | | | |
| 5-fluorotryptophan | ◯ | | | | | | | ◯ | |
| 6-fluorotryptophan | ◯ | | | | | | | ◯ | |
| 1-methyltryptophan | ◯ | | | | | | | ◯ | |
| monoiodotyrosine | ◯ | | | ◯ | | | | | |
| dilodotyrosine | ◯ | | | ◯ | | | | | |
| triiodothyronine | ◯ | | | ◯ | | | | | |
| O-methyltyrosine | ◯ | | | | | | | | |
| 3-nitrotyrosine | ◯ | | | ◯ | | | | | |
| phosphotyrosine | ◯ | | | | | | | | |
| 3-amino-tyrosine | ◯ | ◯ | | ◯ | | | | | |
| cysteineacid | | | | | | | | | ◯ |
| S-benzyl-L-cysteine | ◯ | | | | | | | | ◯ |
| selenocysteine | | | | | | | | | |
| penicillamine | | | | | ◯ | | | | |
| 5-mercaptonorvaline | | | | | ◯ | | | | |
| 6-mercaptonorleucine | | | | | ◯ | | | | |
| 2-amino-7- | | | | | ◯ | | | | |
| mercaptoheptane acid | | | | | | | | | |
| 2-amino-8- | | | | | ◯ | | | | |
| mercaptooctaneacid | | | | | | | | | |
| O-methylserine | | | | | | | | | |
| phosphoserine | | | | | | | | | |
| phosphothreonine | | | | | | | | | |
| γ-hydroxyglutamic | | | ◯ | ◯ | | | | | |
| acid | | | | | | | | | |
| γ-carboxyglutamio | | | ◯ | | | | | | |
| acid | | | | | | | | | |
| N ε-acetyl lysine | | | | | | ◯ | | | |
| homocitrulline | | ◯ | | | | ◯ | | | |
| 2,6-diaminopimelic | | ◯ | ◯ | | | | | | |
| acid | | | | | | | | | |
| alohydroxyllysine | | ◯ | | ◯ | | | | | |
| 2,6-diamino-4- | | ◯ | | | | | | | |
| hexyneacid | | | | | | | | | |
| ω-nitroarginine | | ◯ | | | | | | | |
| ω-amino-arginine | | ◯ | | | | | | | |
| ω-methyl-arginine | | ◯ | | | | | | | |
| citrulline | | ◯ | | | | ◯ | | | |
| 1-methylhistidine | | ◯ | | | | | | ◯ | |
| 3-methylhistidine | | ◯ | | | | | | ◯ | |
| 5-methylhistidine | | ◯ | | | | | | ◯ | |
| 3-amino-5- | ◯ | | | | | | | | |
| phenylpentanoic acid | | | | | | | | | |
| 1,2,3,4- | ◯ | | | | | | ◯ | | |
| tetrahydroisoquinoline- | | | | | | | | | |
| 3-carboxylic acid | | | | | | | | | |
| ω-hydroxy-nor- | | ◯ | | ◯ | | | | | |
| arginine | | | | | | | | | |
| 1-aminocyclopentane- | | | | | | | ◯ | ◯ | |
| 3-carboxylic acid | | | | | | | | | |
| thiazolidine-4- | | | | | | | ◯ | ◯ | ◯ |
| carboxylic acid | | | | | | | | | |
| 5,5-dimethyl-D- | | | | | | | ◯ | ◯ | ◯ |
| thiazolidine-4- | | | | | | | | | |
| carboxylic acid | | | | | | | | | |

As used herein, the term “conservative substitution” means that one amino acid constituting a peptide is replaced with another amino acid in the amino acid family with which it shares the same side chain chemical property. For example, one amino acid is replaced with another amino acid in one of the amino acid families listed in Table 1 to which said amino acid belongs. Examples of conservative substitutions are shown in Table 2.

TABLE 2

| Original | |
|---|---|
| amino acid | Examples of preservative substitution |
| Gly (G) | Ala, ala, MeGly, MeAla, EtGly, Aib, hAla, 2Abu, pGly, Sem, Nle, Nva, 2Apn, Aad, 4Abu, ε Ahx, |
| | Ava, hAla, 2Apn |
| Ala (A) | Gly, Met, ala, met, Aib, Deg, MeGly, MeAla, EtGly, hAla, 2Abu, pGly, Sem, Nle, Nva, 2Apn, Aad |
| Leu (L) | Ile, Val, Met, ile, leu, val, Aml, Deg, Dbg, MeLeu, MeIle, MeVal, Bhl, Bhv, hAla, hIle, 2Abu, tbGly, |
| | tbAla, Nle, Tle, Nva, 2Apn, AIle, pGly, aGly, chGly, cpGly, chAla, cbAla, cpAla |
| Ile (I) | Leu, Val, Met, ile, leu, val, Aml, Deg, Dbg, MeLeu, MeIle, MeVal, Bhl, Bhv, hAla, hIle, 2Abu, |
| | tbGly, tbAla, Nle, Tle, Nva, 2Apn, AIle, pGly, aGly, chGly, cpGly, chAla, cbAla, cpAla |
| Val (V) | Ile, Leu, Met, ile, leu, val, Aml, Deg, Dbg, MeLeu, MeIle, MeVal, Bhl, Bhv, hAla, hIle, 2Abu, |
| | tbGly, tbAla, Nle, Tle, Nva, 2Apn, AIle, pGly, aGly, chGly, cpGly, chAla, cbAla, cpAla |
| Met (M) | Ile, Leu, Val, ile, leu, met, val, MeMet, FoMet, Sem, mMet, Nle, Nva, 2Apn, Aad, Meo, Moo |
| Pro (P) | pro, Amp, Tic, 3Hyp, 4Hyp, 5Hyp, HSPro, Pip, Az3Ca, Az2Ca, Acp3Ca, thz, dtc |
| Phe (F) | Trp, Tyr, phe, tyr, MePhe, MeTyr, Bhf, Bhy, Afp, hPhe, hTyr, fGly, 1npAla, 2npAla, btAla, thAla, |
| | taAla, fAla, stAla, anAla, ffAla, bzCys, iTyr, iiTyr, iiiThy, mTyr, nTyr, pTyr, OmPhe, MmPhe, |
| | PmPhe, OnPhe, MnPhe, PnPhe, OcnPhe, McnPhe, PcnPhe, OcPhe, McPhe, PcPhe, OfPhe, |
| | MfPhe, PfPhe, ObPhe, MbPhe, PbPhe, OiPhe, MiPhe, PiPhe, OpcPhe, MpcPhe, OpfPhe, |
| | MpfPhe, PmmfPhe, 5fPhe, OtPhe, MtPhe, PtPhe, PaPhe, PamPhe, MaTyr, PmmDopa, Dopa, |
| | phmPhe, Tic |
| Trp (W) | Phe, Tyr, phe, tyr, trp, MePhe, MeTyr, MeTrp, Bhf, Bhy, Bhw, Afp, hPhe, hTyr, hTrp, fGly, |
| | 1npAla, 2npAla, btAla, thAla, taAla, fAla, stAla, anAla, ffAla, bzCys, iTyr, iiTyr, iiiThy, mTyr, nTyr, |
| | pTyr, OmPhe, MmPhe, PmPhe, OnPhe, MnPhe, PnPhe, OcnPhe, McnPhe, PcnPhe, OcPhe, |
| | McPhe, PcPhe, OfPhe, MfPhe, PfPhe, ObPhe, MbPhe, PbPhe, OiPhe, MiPhe, PiPhe, OpcPhe, |
| | MpcPhe, OpfPhe, MpfPhe, PmmfPhe, 5fPhe, OtPhe, MtPhe, PtPhe, PaPhe, PamPhe, MaTyr, |
| | PmmDopa, Dopa, phmPhe, 5fTyp, 6fTyp, 1mTyp, Tic |
| Tyr (Y) | Phe, Trp, phe, tyr, MePhe, MeTyr, Bhf, Bhy, Afp, hPhe, hTyr, fGly, 1npAla, 2npAla, btAla, thAla, |
| | taAla, fAla, stAla, anAla, ffAla, bzCys, iTyr, iiTyr, iiiThy, mTyr, nTyr, pTyr, OmPhe, MmPhe, |
| | PmPhe, OnPhe, MnPhe, PnPhe, OcnPhe, McnPhe, PcnPhe, OcPhe, McPhe, PcPhe, OfPhe, |
| | MfPhe, PfPhe, ObPhe, MbPhe, PbPhe, OiPhe, MiPhe, PiPhe, OpcPhe, MpcPhe, OpfPhe, |
| | MpfPhe, PmmfPhe, 5fPhe, OtPhe, MtPhe, PtPhe, PaPho, PamPhe, MaTyr, PmmDopa, Dopa, |
| | phmPhe, Tic |
| Cys (C) | cys, MeCys, Bhcp, Pen, Ncy, HSPro, NHSEtGly, Ser, Ala, Met, Leu, Ile, Val |
| Ser (S) | Thr, ser, thr, MeSer, MeThr, Bhs, Bht, bSer, bThr, hSer, hThr, alThr, pSer, pThr |
| Thr (T) | Ser, ser, thr, MeSer, MeThr, Bhs, Bht, bSer, bThr, hSer, hThr, alThr, pSer, pThr |
| Asn (N) | Gln, asn, gln, MeAsn, MeGln, EtAsn, hGln, bAsn, bGln, Bhq, Asp, Glu |
| Gln (Q) | Asn, asn, gln, MeAsn, MeGln, EtAsn, hGln, bAsn, bGln, Bhq, Asp, Glu |
| Asp (D) | Glu, asp, glu, MeAsp, MeGlu, bGlu, bAsp, hGlu, Bhe, iAsp, iGlu, Cya, Aad, β Aad, hoGlu, caGlu, |
| | Asn, Gln |
| Glu (E) | Asp, asp, glu, MeAsp, MeGlu, bGlu, bAsp, hGlu, Bhe, iAsp, iGlu, Cya, Aad, β Aad, hoGlu, caGlu, |
| | Asn, Gln |
| Lys (K) | Arg, lys, arg, MeLys, MeArg, hLys, hArg, bLys, bArg, Bhk, Bhr, nArg, HOnArg, Oar, Omr, ntArg, |
| | 5OHLys, AHyl, Orn, 2,4-A2bu, 2,3-A2bu, A2pr, DaHea, 2pyAla, 3pAla, 4pyAla, ppAla, PaPhe, |
| | PamPhe, MaTyr, Cit, Hci |
| Arg (R) | Lys, lys, arg, MeLys, MeArg, hLys, hArg, bLys, bArg, Bhk, Bhr, nArg, HOnArg, Oar, Omr, ntArg, |
| | 5OHLys, AHyl, Orn, 2,4-A2bu, 2,3-A2bu, A2pr, DaHea, 2pyAla, 3pAla, 4pyAla, ppAla, PaPhe, |
| | PamPhe, MaTyr, Cit, Hci |
| His (H) | his, MeHis, hHis, 1mHis, 3mHis, Bhh, 2pyAla, 3pAla, 4pyAla |

### II. Cyclic peptide

The present invention provides a cyclic peptide which has an activity to specifically bind to human CTLA-4 (hereinafter referred to as the cyclic peptide of the present invention).

CTLA-4 is a known membrane protein, and its amino acid sequence is also known. A representative amino acid sequence of human CTLA-4 (NCBI Reference Sequence: NP_005205.2) is depicted in SEQ ID NO: 1. In the amino acid sequence represented by SEQ ID NO: 1, the 1^{st} to 35^{th} residues correspond to the signal peptide, and the 36^{th} to 223^{rd} residues correspond to the mature human CTLA-4. In addition, in the amino acid sequence represented by SEQ ID NO: 1, the 162^{nd} to 182^{nd} residues correspond to the transmembrane region, and the 36^{th} to 161^{st} residues correspond to the extracellular region. Preferably, the cyclic polypeptide of the present invention specifically binds to the extracellular region of mature human CTLA-4.

The term “specific binding” of a cyclic peptide to antigen X means that the binding of the cyclic peptide to antigen X is selective and distinguishable from undesired or nonspecific interactions.

The dissociation constant (Kd value) for the binding affinity of the cyclic peptide of the present invention to human CTLA-4 is generally 1×10⁻⁴ M or less (for example, 1×10⁻⁵ M or less, 1×10⁻⁶ M or less, 1×10⁻⁷ M or less or 1×10⁻⁸ M or less).

The binding affinity can be determined by using, for example, surface plasmon resonance (BIAcore (trademark)) analysis or the like. The binding affinity is preferably measured by BLItz system (Fortebio) using SA sensor chip immobilized with biotinylated CTLA-4 protein according to the Example below.

In a preferred embodiment, the cyclic peptide of the present invention has an activity to inhibit the binding between human CTLA-4 and human CD80.

CD80 is one of the ligands for CTLA-4, and also called B7-1. A representative amino acid sequence of human CD80 (NCBI Reference Sequence: NP_005182.1) is depicted in SEQ ID NO: 2. In the amino acid sequence represented by SEQ ID NO: 2, the 1^{st} to 34^{th} residues correspond to the signal peptide, and the 35^{th} to 288^{th} residues correspond to the mature human CD80. In the amino acid sequence represented by SEQ ID NO: 2, the 243^{rd} to 263^{nd} residues correspond to the transmembrane region, and the 35^{th} to 242^{nd} residues correspond to the extracellular region. The polypeptide of the present invention preferably has an activity to inhibit the binding between the extracellular region of mature human CTLA-4 and extracellular region of human CD80.

The cyclic peptide of the present invention has an IC₅₀ value of, for example, 1×10⁻³ M or less (preferably 1×10⁻⁴ M or less, 1×10′⁵ M or less, 1×10⁻⁶ M or less or 1×10⁻⁷ M or less) with respect to its inhibition of binding between human CTLA-4 and human CD80. IC₅₀ can be determined by using, for example, competitive ELISA, surface plasmon resonance (BIAcore (trademark)) analysis or the like. IC₅₀ value for the cyclic peptide of the present invention with respect to its inhibition of binding between human CTLA-4 and human CD80 is preferably measured by CTLA4:B7-1 [Biotinylated] Inhibitor Screening Assay Kit (BPS Bioscience) according to the Example below.

The cyclic peptide of the present invention is characterized in that it comprises the amino acid sequence represented formula (I):

X₁-His-Pro-X₄-Leu-X₆-X₇-X₈-Ser-X₁₀-His-Phe (I)
in the cycle, wherein X₁, X₄, X₆, X₇, X₈ and X₁₀ are each independently any amino acid.

The present invention has been completed on the basis of a finding that cyclic peptides comprising the consensus sequence represented by formula (I) above in the cycle specifically bind to human CTLA-4 and inhibit the binding between human CTLA-4 and human CD80.

As used herein, the term “cyclic peptide” means a peptide in which two amino acids within the peptide are bonded to form a cyclic structure in whole or in part. The cyclic peptide may have a molecular structure other than a cyclic structure, a chain structure in which amino acids are linked by peptide bonds, or a structure other than the peptide structure. The term “cyclic structure” means, in a linear peptide, a closed ring structure formed intramolecularly by bonding, directly or via a linker or the like, of two amino acids separated from each other by plural amino acid residues (e.g., 10 or more amino acid residues). The term “separated from each other by 10 or more amino acid residues” means that at least 10 residues of amino acids exist between two amino acids.

In the cyclic peptide of the present invention, the number of amino acids constituting a cyclic structure is not particularly limited, but is, for example, 12 or more, 13 or more, or 14 or more. The upper limit of the number of amino acids constituting the cyclic structure is not particularly limited as long as the cyclic peptide has an activity to specifically bind to human CTLA-4, but is, for example, 30 or less, 25 or less, 20 or less, or 16 or less.

The number of amino acids constituting a cyclic structure is, for example, 12 or more and 30 or less, 12 or more and 25 or less, 12 or more and 20 or less, 12 or more and 16 or less. The number of amino acids constituting a cyclic structure is, for example, 12, 13, 14, 15 or 16, and preferably 14.

The ring-closing structure in the cyclic structure is formed by an intramolecular bond between two amino acids in the peptide, although it is not particularly limited. Preferably, the ring-closing structure is formed by the covalent bonding of two amino acids, either directly or indirectly via a suitable bridged structure (the bridged structure does not contain any amino acids). Examples of a covalent bond between two amino acids include, but are not limited to, disulfide bond, peptide bond, amide bond (lactam bridge), alkyl bond, alkenyl bond, ester bond, thioester bond, ether bond, thioether bond, phosphonate ether bond, azo bond, C—S—C bond, C—N—C bond, C═N—C bond, carbamoyl bond, urea bond, thiourea bond, amine bond, thioamide bond and the like. When two amino acids are bonded in the main chain of the amino acids, the ring-closing structure is formed by peptide bond, but the covalent bond between two amino acids may be formed by bonding between the side chains of two amino acids, between the side chain and the main chain or the like.

The cyclic structure may be formed by not only a covalent bond between the N-terminal amino acid and the C-terminal amino acid of a linear peptide, but also a covalent bond between the terminal amino acid and non-terminal amino acid, or a covalent bond between non-terminal amino acids.

In one embodiment, the cyclic peptide of the present invention comprises the amino acid sequence represented by formula (II):

X₀-X₁-His-Pro-X₄-Leu-X₆-X₇-X₈-Ser-X₁₀-His-Phe-X₀₀ (II)
wherein X₀, X₁, X₄, X₆, X₇, X₈, X₁₀ and X₀₀ are each independently any amino acid, and it is cyclized by an intramolecular bond between X₀ and X₀₀.

In one embodiment, the cyclic peptide of the present invention comprises the amino acid sequence represented by formula (III):

(Y)ₘ-X₀-X₁-His-Pro-X₄-Leu-X₆-X₇-X₈-Ser-X₁₀-His-Phe-X₀₀-(Z)ₙ (III)
wherein X₀, X₁, X₄, X₆, X₇, X₈, X₁₀ and X₀₀ are each independently any amino acid,
(Y)m is an amino acid sequence having a length of m amino acid(s),
(Z)n is an amino acid sequence having a length of n amino acid(s),
m is any integer selected from the group consisting of 0, 1 and 2,
n is any integer selected from the group consisting of 0, 1 and 2, and
it is cyclized by an intramolecular bond between X₀ and X₀₀.

In formulae (II) and (III), the mode of intramolecular bond between X₀ and X₀₀ is not particularly limited, but can be, for example, those described above as a covalent bond between two amino acids forming a ring-closing structure.

The following are examples of specific embodiments of cyclization by an intramolecular bond between X₀ and X₀₀.
(1) X₀ and X₀₀ are each independently an amino acid having a side chain containing thiol group, and intramolecular disulfide bond is formed between the side chain thiol group in X₀ and the side chain thiol group in X₀₀.
(2) X₀ and X₀₀ are each independently any amino acid, and amino bond (peptide bond) is formed between the main chain amino group in X₀ and the main chain carboxyl group in X₀₀.
(3) One of X₀ and X₀₀ is an amino acid having a side chain containing amino group and the other is an amino acid having a side chain containing carboxy group, and amide bond (lactam bridge) is formed between the side chain amino group and the side chain carboxyl group.
(4) X₀ and X₀₀ are each independently an amino acid having a side chain containing thiol group, and thioether bond via mesitylene is formed between the side chain thiol group in X₀ and the side chain thiol group in X₀₀ (Nature Chem. Bio. 2009, 5, 502).
(5) X₀ is any amino acid, X₀₀ is an amino acid having a side chain containing amino group, and the main chain amino group in X₀ and the side chain amino group in X₀₀ are crosslinked by disuccinimidyl glutarate (DSG) (J. Am. Chem. Soc. 2002, 124, 9972).
(6) One of X₀ and X₀₀ is an amino acid having chloroacetyl group and the other is an amino acid having a side chain containing thiol group, and thioether bond is formed between the chloroacetyl group and the thiol group (US2010/168380 A1; WO2017/217545 A1).
(7) One of X₀ and X₀₀ is an amino acid having ethynyl group (—C≡CH) and the other is an amino acid having azido group (˜N3), and X₀ and X₀₀ are crosslinked via triazole ring by an intramolecular Click reaction between the ethynyl group and the azido group (US2010/168380 A1; WO2017/217545 A1).
(8) One of X₀ and X₀₀ is an amino acid having benzylamino group and the other is an amino acid having 5-hydroxyindole, and X₀ and X₀₀ are crosslinked via a fluorescent structure by an intramolecular cyclization reaction between the benzylamino group and the 5-hydroxyindole (ChemBioChem 10, 1469-1472 (2009); US2010/168380 A1; WO2017/217545 A1).
(9) One of X₀ and X₀₀ is an amino acid having —C≡C—CH₂—X, —C═C—CH₂—X or —Ar—CH₂—X, wherein Ar is an aromatic ring which optionally has a substituent, X is a leaving group, and examples of the leaving group include a halogen atom including Cl, Br and I, or the like, the other is an amino acid having a side chain containing thiol group, and X₀ and X₀₀ are crosslinked via a thioether bond by a condensation reaction between said —C═C—CH₂—X, —C═C—CH₂—X or —Ar—CH₂—X and the thiol group (US2013/316910 A1).
(10) X₀ and X₀₀ are crosslinked via a covalent bone by the method described in US2015/0080549 A1, US2015/050269 A1 or the like.

In one embodiment, X₀ and X₀₀ are each independently an amino acid having a side chain containing thiol group. The amino acid having a side chain containing thiol group is preferably cysteine (C). In this embodiment, an intramolecular bond may be formed between the side chain thiol group in X₀ and the side chain thiol group in X₀₀ (e.g., (1) or (4) above). Preferably, a disulfide bond is formed between the side chain thiol group in X₀ and the side chain thiol group in X₀₀.

In one embodiment, the cyclic peptide of the present invention comprises the amino acid sequence represented by formula (IIA):

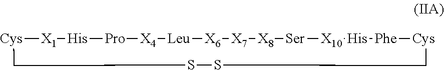

wherein X₁, X₄, X₆, X₇, X₈ and X₁₀ are each independently any amino acid.

In one embodiment, the cyclic peptide of the present invention comprises the amino acid sequence represented by formula (IIIA):

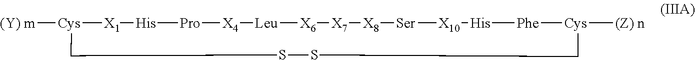

wherein X₁, X₄, X₆, X₇, X₈ and X₁₀ are each independently any amino acid,
(Y)m is an amino acid sequence having a length of m amino acid(s),
(Z)n is an amino acid sequence having a length of n amino acid(s),
m is any integer selected from the group consisting of 0, 1 and 2, and
n is any integer selected from the group consisting of 0, 1 and 2.

In formulae (I), (II), (IIA), (III) and (IIIA),
X₆ is, preferably, a hydrophobic amino acid, a neutral hydrophilic amino acid, a basic amino acid, an amino acid having a side chain containing imino group, an amino acid having a side chain containing amido group or an amino acid having a side chain containing amino group,
X₇ is, preferably, a hydrophobic amino acid, a neutral hydrophilic amino acid or an amino acid having a side chain containing hydroxyl group. More preferably, X₆ is Leu, Pro, Gln, Lys or Arg, and X₇ is Val, Leu, Ile or Thr. Most preferably, X₆ is Pro, and X₇ is Ile.

In one embodiment, the cyclic peptide of the present invention comprises the amino acid sequence represented by formula (IB):

X₁-His-Pro-X₄-Leu-Pro-Ile-X₈-Ser-X₁₀-His-Phe (IB)
in the cycle,
wherein X₁, X₄, X₈ and X₁₀ are each independently any amino acid.

In one embodiment, the cyclic peptide of the present invention comprises the amino acid sequence represented by formula (IIB):

X₀-X₁-His-Pro-X₄-Leu-Pro-Ile-X₈-Ser-X₁₀-His-Phe-X₀₀ (IIB)
wherein X₀, X₁, X₄, X₈, X₁₀ and X₀₀ are each independently any amino acid, and the cyclic peptide is cyclized by an intramolecular bond between X₀ and X₀₀.

In one embodiment, the cyclic peptide of the present invention comprises the amino acid sequence represented by formula (IIIB):

(Y)ₘ-X₀-X₁-His-Pro-X₄-Leu-Pro-Ile-X₈-Ser-X₁₀-His-Phe-X₀₀-(Z)n (IIIB)
wherein X₀, X₁, X₄, X₈, X₁₀ and X₀₀ are each independently any amino acid,
(Y)m is an amino acid sequence having a length of m amino acid(s),
(Z)n is an amino acid sequence having a length of n amino acid(s),
m is any integer selected from the group consisting of 0, 1 and 2,
n is any integer selected from the group consisting of 0, 1 and 2, and
wherein the peptide is cyclized by an intramolecular bond.

In one embodiment, the cyclic peptide of the present invention comprises the amino acid sequence represented by formula (IIAB):

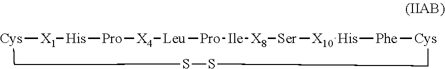

wherein, X₁, X₄, X₈ and X₁₀ are each independently any amino acid.

In one embodiment, the cyclic peptide of the present invention comprises the amino acid sequence represented by formula (IIIAB):

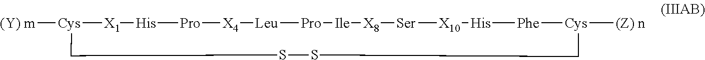

wherein X₁, X₄, X₈, and X₁₀ are each independently any amino acid,
(Y)m is an amino acid sequence having a length of m amino acid(s),
(Z)n is an amino acid sequence having a length of n amino acid(s),
m is any integer selected from the group consisting of 0, 1 and 2, and
n is any integer selected from the group consisting of 0, 1 and 2.

In formulae (I), (IB), (II), (IIA), (IIB), (IIAB), (III), (IIIA), (IIIB) and (IIIAB),
X₁ is, preferably, a hydrophobic amino acid, a neutral hydrophilic amino acid, a basic amino acid, an aromatic amino acid, an amino acid having a side chain containing a heterocycle, an amino acid having a side chain containing sulfur atom, an amino acid having a side chain containing imino group, an amino acid having a side chain containing hydroxyl group, an amino acid having a side chain containing amido group, an amino acid having a side chain containing amino group or an amino acid having a side chain containing thiol group,
X₄ is, preferably, a hydrophobic amino acid, a neutral hydrophilic amino acid, a basic amino acid, an aromatic amino acid, an amino acid having a side chain containing a heterocycle, an amino acid having a side chain containing sulfur atom, an amino acid having a side chain containing hydroxyl group, an amino acid having a side chain containing amido group, an amino acid having a side chain containing amino group or an amino acid having a side chain containing thiol group,
X₈ is, preferably, a hydrophobic amino acid, a neutral hydrophilic amino acid, a basic amino acid, an aromatic amino acid, an amino acid having a side chain containing a heterocycle, an amino acid having a side chain containing sulfur atom, an amino acid having a side chain containing hydroxyl group, an amino acid having a side chain containing amido group or an amino acid having a side chain containing amino group,
X₁₀ is, preferably, a hydrophobic amino acid, a neutral hydrophilic amino acid, a basic amino acid, an acidic amino acid, an aromatic amino acid, an amino acid having a side chain containing a heterocycle, an amino acid having a side chain containing sulfur atom, an amino acid having a side chain containing imino group, an amino acid having a side chain containing hydroxyl group, an amino acid having a side chain containing amido group, an amino acid having a side chain containing amino group, an amino acid having a side chain containing carboxy group or an amino acid having a side chain containing thiol group.

More preferably, X₁ is Trp, Phe, Val, Leu, Ile, Met, Pro, Ala, Gly, Ser, Gln, Lys, Arg or Cys,
X₄ is Trp, Phe, Tyr, Val, Leu, Met, Ala, Gly, Ser, Gln, His or Cys,
X₈ is Trp, Phe, Tyr, Val, Leu, Ile, Met, Ala, Ser, Gln, Lys, Arg or His, and
X₁₀ is Trp, Phe, Tyr, Val, Leu, Ile, Met, Pro, Ala, Gly, Ser, Thr, Gln, Asp, Lys, Arg, His or Cys.

In formulae (III), (IIIA), (IIIB) and (IIIAB),
(Z)n does not exist, when n is 0,
(Z)n is an amino acid represented by X₊₁, when n is 1,
(Z)n consists of an amino acid sequence represented by X₊₁-X₊₂, when n is 2, and X₊₁ and X₊₂ are each independently any amino acid. Preferably n is 2.

Preferably, X₊₁ is a hydrophobic amino acid, a neutral hydrophilic amino acid, a basic amino acid, an acidic amino acid, an aromatic amino acid, an amino acid having a side chain containing a heterocycle, an amino acid having a side chain containing sulfur atom, an amino acid having a side chain containing imino group, an amino acid having a side chain containing hydroxyl group, an amino acid having a side chain containing amido group, an amino acid having a side chain containing amino group or an amino acid having a side chain containing thiol group, X₊₂ is a hydrophobic amino acid, a neutral hydrophilic amino acid, a basic amino acid, an aromatic amino acid, an amino acid having a side chain containing a heterocycle, an amino acid having a side chain containing sulfur atom, an amino acid having a side chain containing hydroxyl group, an amino acid having a side chain containing amido group, an amino acid having a side chain containing amino group or an amino acid having a side chain containing thiol group. More preferably, X₊₁ is Trp, Val, Leu, Met, Pro, Ala, Gly, Ser, Gln, Glu, Arg or Cys, and X₊₂ is Trp, Phe, Tyr, Val, Leu, Ile, Met, Ala, Gly, Ser, Gln, Lys, Arg, His or Cys.

In formulae (III), (IIIA), (IIIB) and (IIIAB),
(Y)m does not exist, when m is 0,
(Y)m is an amino acid represented by X₋₁, when m is 1,
(Y)m consists of an amino acid sequence represented by X₋₂-X₋₁ when m is 2, and X₋₁ and X₋₂ are each independently any amino acid. Preferably, m is 1 or 2.

Preferably, X₋₁ is a hydrophobic amino acid, a neutral hydrophilic amino acid, basic amino acid, an acidic amino acid, an amino acid having a side chain containing hydroxyl group, an amino acid containing amino group or an amino acid having a side chain containing carboxy group, and X₋₂ is a hydrophobic amino acid. More preferably, X₋₁ is Gly, Ser, Lys or Asp, and X₋₂ is Gly.

In one embodiment,
m is 2,
X₋₁ is a hydrophobic amino acid, a neutral hydrophilic amino acid, an acidic amino acid, an amino acid having a side chain containing hydroxyl group or an amino acid having a side chain containing carboxyl group, and
X₋₂ is a hydrophobic amino acid.

In said embodiment, preferably,
X₋₂ is Gly, and
X₋₁ is Gly, Ser or Asp.

In a specific embodiment, the amino acid sequence represented by formula (I) (which includes “a partial sequence of the amino acid sequence represented by formula (II)” consisting of the amino acid sequence represented by formula (I), and “a partial sequence of the amino acid sequence represented by formula (III)” consisting of the amino acid sequence represented by formula (I)) consists of
(1a) an amino acid sequence from position 4 to position 15 in the amino acid sequence represented by any of SEQ ID NOs: 30-211, or
(2a) an amino acid sequence having a substitution (preferably, conservative substitution) at least at one amino acid (e.g., at 1-6, 1-5, 1-4, 1-3, 1 or 2 amino acids) corresponding to one selected from X₁, X₄, X₆, X₁, X₈ and X₁₀ in an amino acid sequence from position 4 to position 15 in the amino acid sequence represented by any of SEQ ID NOs: 30-211 (i.e., the amino acids at positions 4, 7, 9, 10, 11 and 13 in the amino acid sequence represented by any of SEQ ID NOs: 30-211).

Preferably, the amino acid sequence of (2a) above has a substitution (preferably, conservative substitution) at least at one amino acid (e.g., at 1-4, 1-3, 1 or 2 amino acids) corresponding to one selected from X₁, X₄, X₈ and X₁₀ in an amino acid sequence from position 4 to position 15 in the amino acid sequence represented by any of SEQ ID NOs: 30-211 (i.e., the amino acids at positions 4, 7, 11 and 13 in the amino acid sequence represented by any of SEQ ID NOs: 30-211).

In a specific embodiment, the amino acid sequence represented by formula (II) (which includes “a partial sequence of the amino acid sequence represented by formula (III)” consisting of the amino acid sequence represented by formula (II)) consists of
(1b) an amino acid sequence from position 3 to position 16 in the amino acid sequence represented by any of SEQ ID NOs: 30-211, or
(2b) an amino acid sequence having a substitution (preferably, conservative substitution) at least at one amino acid (e.g., at 1-6, 1-5, 1-4, 1-3, 1 or 2 amino acids) corresponding to one selected from X₁, X₄, X₆, X₇, X₈ and X₁₀ in an amino acid sequence from position 3 to position 16 in the amino acid sequence represented by any of SEQ ID NOs: 30-211 (i.e., the amino acids at positions 4, 7, 9, 10, 11 and 13 in the amino acid sequence represented by any of SEQ ID NOs: 30-211).

Preferably, the amino acid sequence of (2b) above has a substitution (preferably, conservative substitution) at least at one amino acid (e.g., at 1-4, 1-3, 1 or 2 amino acids) corresponding to one selected from X₁, X₄, X₈ and X₁₀ in an amino acid sequence from position 3 to position 16 in the amino acid sequence represented by any of SEQ ID NOs: 30-211 (i.e., the amino acids at positions 4, 7, 11 and 13 in the amino acid sequence represented by any of SEQ ID NOs: 30-211).

In said embodiment, the side chain thiol group in the Cys at position 1 and the side chain thiol group in the Cys at position 14 in the amino acid sequence of (1b) or (2b) form an intramolecular bond. The binding manner is not particularly limited, but it is preferably a disulfide bond or a thioether bond via mesitylene (Nature Chem. Bio. 2009, 5, 502), and more preferably a disulfide bond.

In a specific embodiment, the amino acid sequence represented by formula (III) consists of
(1c) an amino acid sequence from position 3 to position 18 in the amino acid sequence represented by any of SEQ ID NOs: 30-211, or
(2c) an amino acid sequence having a substitution (preferably, conservative substitution) at least at one amino acid (e.g., at 1-8, 1-7, 1-6, 1-5, 1-4, 1-3, 1 or 2 amino acids) corresponding to one selected from X₁, X₄, X₆, X₇, X₈, X₁₀, X₊₁ and X₊₂ in an amino acid sequence from position 3 to position 18 in the amino acid sequence represented by any of SEQ ID NOs: 30-211 (i.e., the amino acids at positions 4, 7, 9, 10, 11, 13, 17 and 18 in the amino acid sequence represented by any of SEQ ID NOs: 30-211).

Preferably, the amino acid sequence of (2c) above has a substitution (preferably, conservative substitution) at least at one amino acid (e.g., at 1-6, 1-5, 1-4, 1-3, 1 or 2 amino acids) corresponding to one selected from X₁, X₄, X₈, X₁₀, X₊₁ and X₊₂ in an amino acid sequence from position 3 to position 18 in the amino acid sequence represented by any of SEQ ID NOs: 30-211 (i.e., the amino acids at positions 4, 7, 11, 13, 17 and 18 in the amino acid sequence represented by any of SEQ ID NOs: 30-211).

In said embodiment, the side chain thiol group in the Cys at position 1 and the side chain thiol group in the Cys at position 14 in the amino acid sequence of (1c) or (2c) form an intramolecular bond. The binding manner is not particularly limited, but it is preferably a disulfide bond or a thioether bond via mesitylene (Nature Chem. Bio. 2009, 5, 502), and more preferably a disulfide bond.

In a specific embodiment, the amino acid sequence represented by formula (III) consists of
(1d) the amino acid sequence represented by any of SEQ ID NOs: 30-211, or
(2d) an amino acid sequence having a substitution (preferably, conservative substitution) at least at one amino acid (e.g., at 1-10, 1-9, 1-8, 1-7, 1-6, 1-5, 1-4, 1-3, 1 or 2 amino acids) corresponding to one selected from X₋₂, X₋₁, X₁, X₄, X₆, X₇, X₈,
X₁₀, X₊₁ and X₊₂ in the amino acid sequence represented by any of SEQ ID NOs: 30-211 (i.e., the amino acids at positions 1, 2, 4, 7, 9, 10, 11, 13, 17 and 18 in the amino acid sequence represented by any of SEQ ID NOs: 30-211).

Preferably, the amino acid sequence of (2d) above has a substitution (preferably, conservative substitution) at least at one amino acid (e.g., at 1-8, 1-7, 1-6, 1-5, 1-4, 1-3, 1 or 2 amino acids) corresponding to one selected from X₋₂, X₋₁, X₁, X₄, X₈, X₁₀, X₊₁ and X₊₂ in the amino acid sequence represented by any of SEQ ID NOs: 30-211 (i.e., the amino acids at positions 1, 2, 4, 7, 11, 13, 17 and 18 in the amino acid sequence represented by any of SEQ ID NOs: 30-211).

In one embodiment, the amino acid sequence of (2d) above preferably has a substitution (preferably, conservative substitution) at least at one amino acid (e.g., at 1-6, 1-5, 1-4, 1-3, 1 or 2 amino acids) corresponding to one selected from X₁, X₄, X₈, X₁₀, X₊₁ and X₊₂ in the amino acid sequence represented by any of SEQ ID NOs: 30-211 (i.e., the amino acids at positions 4, 7, 11, 13, 17 and 18 amino acids in the amino acid sequence represented by any of SEQ ID NOs: 30-211).

In said embodiment, the side chain thiol group in the Cys at position 3 and the side chain thiol group in the Cys at position 16 in the amino acid sequence of (1 d) or (2d) form an intramolecular bond. The binding manner is not particularly limited, but it is preferably a disulfide bond or a thioether bond via mesitylene (Nature Chem. Bio. 2009, 5, 502), and more preferably a disulfide bond.

In one embodiment, the cyclic peptide of the present invention comprises the amino acid sequence represented by formula (II):

X₀-X₁-His-Pro-X₄-Leu-X₆-X₇-X₈-Ser-X₁₀-His-Phe-X₀₀ (II)
wherein
X₁ is Trp, Phe, Val, Leu, Ile, Met, Pro, Ala, Gly, Ser, Gln, Lys, Arg or Cys,
X₄ is Trp, Phe, Tyr, Val, Leu, Met, Ala, Gly, Ser, Gln, His or Cys,
X₆ is Leu, Pro, Gln, Lys or Arg,
X₇ is Val, Leu, Ile or Thr,
X₈ is Trp, Phe, Tyr, Val, Leu, Ile, Met, Ala, Ser, Gln, Lys, Arg or His,
X₁₀ is Trp, Phe, Tyr, Val, Leu, Ile, Met, Pro, Ala, Gly, Ser, Thr, Gln, Asp, Lys, Arg, His or Cys,
X₀ and X₀₀ are each independently any amino acid, and
the peptide is cyclized by an intramolecular bond between X₀ and X₀₀.

In said embodiment, preferably, X₆ is Pro, and X₇ is Ile.

As a specific embodiment of the cyclization by an intramolecular bond between X₀ and X₀₀, those described above can be exemplified. Preferably, X₀ and X₀₀ are each independently an amino acid having a side chain containing thiol group, and more preferably, X₀ and X₀₀ are Cys. The binding manner between the side chain thiol group in X₀ and the side chain thiol group in X₀₀ is not particularly limited, but it is preferably a disulfide bond or a thioether bond via mesitylene (Nature Chem. Bio. 2009, 5, 502), and more preferably a disulfide bond.

In one embodiment, the cyclic peptide of the present invention comprises the amino acid sequence represented by formula (II):

X₀-X₁-His-Pro-X₄-Leu-X₆-X₁-X₈-Ser-X₁₀-His-Phe-X₀₀ (II)
wherein
the partial sequence (X₁-His-Pro-X₄-Leu-X₆-X₇-X₈-Ser-X₁₀-His-Phe) consists of
(1e) an amino acid sequence from the 4th to 15th positions in the amino acid sequence represented by any of SEQ ID NOs: 30-211, or
(2e) an amino acid sequence having a substitution (preferably, conservative substitution) at least at one amino acid (e.g., at 1-6, 1-5, 1-4, 1-3, 1 or 2 amino acids) corresponding to one selected from X₁, X₄, X₆, X₇, X₈ and X₁₀ in an amino acid sequence from position 4 to position 15 in the amino acid sequence represented by any of SEQ ID NOs: 30-211 (i.e., the amino acids at positions 4, 7, 9, 10, 11 and 13 in the amino acid sequence represented by any of SEQ ID NOs: 30-211),

X₀ and X₀₀ are each independently any amino acid, and
the peptide is cyclized by an intramolecular bond between X₀ and X₀₀.

Preferably, the amino acid sequence of (2e) above has a substitution (preferably, conservative substitution) at least at one amino acid (e.g., at 1-4, 1-3, 1 or 2 amino acids) corresponding to one selected from X₁, X₄, X₈ and X₁₀ in an amino acid sequence from position 4 to position 15 in the amino acid sequence represented by any of SEQ ID NOs: 30-211 (i.e., the amino acids at positions 4, 7, 11 and 13 in the amino acid sequence represented by any of SEQ ID NOs: 30-211).

As a specific embodiment of the cyclization by an intramolecular bond between X₀ and X₀₀, those described above can be exemplified. Preferably, X₀ and X₀₀ are each independently an amino acid having a side chain containing thiol group, and more preferably, X₀ and X₀₀ are Cys. The binding manner between the side chain thiol group in X₀ and the side chain thiol group in X₀₀ is not particularly limited, but it is preferably a disulfide bond or a thioether bond via mesitylene (Nature Chem. Bio. 2009, 5, 502), and more preferably a disulfide bond.

In one embodiment, the cyclic peptide of the present invention comprises the amino acid sequence represented by formula (III):

(Y)m-X₀-X₁-His-Pro-X₄-Leu-X₆-X₁-X₈-Ser-X₁₀-His-Phe-X₀₀-(Z)n (III)
wherein
X₁ is Trp, Phe, Val, Leu, Ile, Met, Pro, Ala, Gly, Ser, Gln, Lys, Arg or Cys,
X₄ is Trp, Phe, Tyr, Val, Leu, Met, Ala, Gly, Ser, Gln, His or Cys,
X₆ is Leu, Pro, Gln, Lys or Arg,
X₇ is Val, Leu, Ile or Thr,
X₈ is Trp, Phe, Tyr, Val, Leu, Ile, Met, Ala, Ser, Gln, Lys, Arg or His,
X₁₀ is Trp, Phe, Tyr, Val, Leu, Ile, Met, Pro, Ala, Gly, Ser, Thr, Gln, Asp, Lys, Arg, His or Cys,
(Y)m is an amino acid sequence having a length of m amino acid(s),
(Z)n is an amino acid sequence having a length of n amino acid(s),
m is any integer selected from the group consisting of 0, 1 and 2,
n is any integer selected from the group consisting of 0, 1 and 2,
X₀ and X₀₀ are each independently any amino acid, and
the peptide is cyclized by an intramolecular bond between X₀ and X₀₀.

In said embodiment, preferably, X₆ is Pro, and X₇ is Ile.

As a specific embodiment of the cyclization by an intramolecular bond between X₀ and X₀₀, those described above can be exemplified. Preferably, X₀ and X₀₀ are each independently an amino acid having a side chain containing thiol group, and more preferably, X₀ and X₀₀ are Cys. The binding manner between the side chain thiol group in X₀ and the side chain thiol group in X₀₀ is not particularly limited, but it is preferably a disulfide bond or a thioether bond via mesitylene (Nature Chem. Bio. 2009, 5, 502), and more preferably a disulfide bond.

n is preferably 2, (Z)n consists of an amino acid sequence represented by X₊₁-X₊₂, and X₊₁ and X₊₂ are each independently any amino acid. Preferably, X₊₁ is Trp, Val, Leu, Met, Pro, Ala, Gly, Ser, Gln, Glu, Arg or Cys, X₊₂ is Trp, Phe, Tyr, Val, Leu, Ile, Met, Ala, Gly, Ser, Gln, Lys, Arg, His or Cys.

m is preferably 2, (Y)m consists of an amino acid sequence represented by X₋₂-X₋₁, and X₋₁ and X₋₂ are each independently any amino acid. Preferably, X₋₁ is Gly, Ser or Asp, and X₋₂ is Gly.

In one embodiment, the cyclic peptide of the present invention comprises an amino acid sequence represented by formula (III):

(Y)m-X₀-X₁-His-Pro-X₄-Leu-X₆-X₇-X₈-Ser-X₁₀-His-Phe-X₀₀-(Z)n (III)
wherein
the partial sequence (X₁-His-Pro-X₄-Leu-X₆-X₇-X₈-Ser-X₁₀-His-Phe) consists of
(1 f) an amino acid sequence from position 4 to position 15 in the amino acid sequence represented by any of SEQ ID NOs: 30-211, or
(2f) an amino acid sequence having a substitution (preferably, conservative substitution) at least at one amino acid (e.g., at 1-6, 1-5, 1-4, 1-3, 1 or 2 amino acids) corresponding to one selected from X₁, X₄, X₆, X₇, X₈ and X₁₀ in an amino acid sequence from position 4 to position 15 in the amino acid sequence represented by any of SEQ ID NOs: 30-211 (i.e., the amino acids at positions 4, 7, 9, 10, 11 and 13 in the amino acid sequence represented by any of SEQ ID NOs: 30-216),
(Y)m is an amino acid sequence having a length of m amino acid(s),
(Z)n is an amino acid sequence having a length of n amino acid(s),
m is any integer selected from the group consisting of 0, 1 and 2,
n is any integer selected from the group consisting of 0, 1 and 2,
X₀ and X₀₀ are each independently any amino acid, and
the peptide is cyclized by an intramolecular bond between X₀ and X₀₀.

Preferably, the amino acid sequence of (2f) above has a substitution (preferably, conservative substitution) at least at one amino acid (e.g., at 1-4, 1-3, 1 or 2 amino acids) corresponding to one selected from X₁, X₄, X₈ and X₁₀ in an amino acid sequence from position 4 to position 15 in the amino acid sequence represented by any of SEQ ID NOs: 30-211 (i.e., the amino acids at positions 4, 7, 11 and 13 in the amino acid sequence represented by any of SEQ ID NOs: 30-211).

As a specific embodiment of the cyclization by an intramolecular bond between X₀ and X₀₀, those described above can be exemplified. Preferably, X₀ and X₀₀ are each independently an amino acid having a side chain containing thiol group, and more preferably, X₀ and X₀₀ are Cys. The binding manner between the side chain thiol group in X₀ and the side chain thiol group in X₀₀ is not particularly limited, but it is preferably a disulfide bond or a thioether bond via mesitylene (Nature Chem. Bio. 2009, 5, 502), and more preferably a disulfide bond.

n is preferably 2, (Z)n consists of an amino acid sequence represented by X₊₁-X₊₂, and X₊₁ and X₊₂ are each independently any amino acid. Preferably, X₊₁ is Trp, Val, Leu, Met, Pro, Ala, Gly, Ser, Gln, Glu, Arg or Cys, X₊₂ is Trp, Phe, Tyr, Val, Leu, Ile, Met, Ala, Gly, Ser, Gln, Lys, Arg, His or Cys.

m is preferably 2, (Y)m consists of an amino acid sequence represented by X₋₂-X₋₁, and X₋₁ and X₋₂ are each independently any amino acid. Preferably, X₋₁ is Gly, Ser or Asp, and X₋₂ is Gly.

The length (amino acid length) of the cyclic peptide of the present invention is not particularly limited as long as it has an activity to specifically bind to human CTLA-4, but it is, for example, 12 or more, 13 or more, 14 or more, 15 or more, 16 or more, 17 or more or 18 or more. The upper limit of the length of the cyclic peptide of the present invention is not particularly limited as long as the peptide has an activity to specifically bind to human, but it is, for example, 2000 or less, 1000 or less, 500 or less, 250 or less or 100 or less. In one embodiment, the length (amino acid length) of the cyclic peptide of the present invention is 12, 13, 14, 15, 16, 17 or 18.

The cyclic peptide of the present invention may comprise an additional sequence at N-terminal side and/or C-terminal side of an amino acid sequence represented by formulae (I), (IB), (II), (IIA), (IIB), (IIAB), (III), (IIIA), (IIIB) or (IIIAB). As the additional sequence, for example, an amino acid sequence of a tag that facilitates detection, purification or the like of the peptide can be mentioned. Examples of the tag include, but not limited to, Flag tag, histidine tag, c-Myc tag, HA tag, AU1 tag, GST tag, MBP tag, fluorescence protein tag (e.g., GFP, YFP, RFP, CFP, BFP etc.), immunoglobulin Fc tag and the like.

The cyclic peptide of the present invention is preferably isolated. Being “isolated” means that an operation to remove components other than the component of interest has been applied to the state of natural presence. The purity of the isolated cyclic peptide of the present invention determined by, for example, electrophoresis (e.g., SDS-PAGE, isoelectric focusing electrophoresis (IEF), capillary electrophoresis) or chromatography (e.g., ion exchange or reverse-phase HPLC) and the like is generally 50% or more, preferably 70% or more, more preferably 90% or more, most preferably 95% or more (e.g., substantially 100%). For a method to evaluate the purity of a compound, see Flatman et al., J. Chromatogr. B 848:79-87 (2007).

The cyclic peptide of the present invention may be modified by acylation (e.g., acylation of N-terminal amino group), amidation (e.g., amidation of C-terminal carboxyl group), phosphorylation, methylation, acetylation, adenylation, ADP-ribosylation, esterification, halogenation, glycosylation, PEG addition, alkyl chain addition or the like. In addition, the cyclic peptide of the present invention may be fused with other peptides or proteins.

The cyclic peptide of the present invention may be labeled by a labeling agent. As the labeling agent, for example, biotin, enzyme, fluorescent substance, luminescent substance, radioisotope, toxin or the like may be used. As the enzyme, a stable enzyme having a high specific activity is preferable, for example, β-galactosidase, β-glucosidase, alkaline phosphatase, peroxidase, malate dehydrogenase or the like may be used. As the fluorescent substance, for example, fluorescamine, fluorescein isothiocyanate or the like may be used. As the luminescent substance, for example, luminol, luminol derivative, luciferin, lucigenin or the like may be used. As the radioisotope, for example, ¹²⁵I, ¹³¹I, ³H, ¹⁴C or the like may be used.

The cyclic peptide of the present invention includes the salt form. As a salt of the cyclic peptide, a salt with physiologically acceptable base or acid may be used, and examples of the salt include addition salts with an inorganic acid (hydrochloric acid, hydrobromic acid, hydriodic acid, sulfuric acid, phosphoric acid or the like), addition salts with an organic salt (p-toluenesulfonic acid, methanesulfonic acid, oxalic acid, p-bromophenylsulfonic acid, carboxylic acid, succinic acid, citric acid, benzoic acid, acetic acid or the like), addition salts with an inorganic base (ammonium hydroxide or alkali- or alkaline-earth metal hydroxides, carbonates, bicarbonates or the like), amino acid and the like.

The cyclic peptide of the present invention can be produced by using known peptide synthesis techniques. Examples of a method for synthesizing cyclic peptides include chemical synthesis methods such as solid-phase synthesis method, liquid-phase synthesis method, and hybrid methods; genetic recombination methods; and translation synthesis methods using cell-free translation system.

The synthesis of the cyclic peptide of the present invention by a translation synthesis method using a cell-free translation system can be prepared by preparing a nucleic acid encoding the cyclic peptide, and translating the nucleic acid in the cell-free translation system. When a cyclic peptide cyclized by a disulfide bond is synthesized in a cell-free translation system, an optimized reaction solution supplemented with oxidized glutathione, reduced glutathione, and DsbC, a molecular chaperon may be used (Shimizu et al., (2005) Methods, vol. 36, p. 299-304). A person having ordinary skill in the art can appropriately design a nucleic acid encoding a cyclic peptide based on the amino acid sequence of the cyclic amino acid. The nucleic acid may be DNA or RNA. Natural amino acids as well as non-natural amino acids can be efficiently introduced to the cyclic peptide by using tRNA aminoacylated with non-natural amino acids. The cyclization of the peptide can be performed by know methods, for example, as described above as an example of ring-closing structure in the cyclic structure.

After the synthesis reaction, the cyclic peptide of the present can be isolated or purified by using purification methods generally used in the field of peptide, for example, solvent extraction, distillation, column chromatography, liquid chromatography, recrystallization or the like, in combination.

The cyclic peptide of the present invention can specifically bind to CTLA-4 and inhibit a function of human CTLA-4. Accordingly, the cyclic peptide of the present invention is useful as a CTLA-4 inhibitor (preferably, a human CTLA-4 inhibitor). The present invention provides a CTLA-4 inhibitor (preferably, a human CTLA-4 inhibitor) comprising the cyclic peptide of the present invention described above (hereinafter referred to as the CTLA-4 inhibitor of the present invention). A function of CTLA-4 in a subject (e.g., human) can be inhibited by administering an effective amount of the cyclic peptide of the present invention to the subject. A function of CTLA-4 can be inhibited by contacting CTLA-4 with an effective amount of the cyclic peptide of the invention in vivo or in vitro. The function of CTLA-4 includes binding to its ligands, CD80 or CD86. The CTLA-4 inhibitor of the present invention inhibits binding of CTLA-4 to its ligand (CD80 or CD86) and suppresses the subsequent downstream cascade (biological responses). The CTLA-4 inhibitor of the present invention inhibits binding of CTLA-4 (e.g., human CTLA-4) to CD80 (e.g., human CD80) and/or binding of CTLA-4 (e.g., human CTLA-4) to CD86 (e.g., human CD86), preferably inhibits binding of CTLA-4 (e.g., human CTLA-4) to CD80 (e.g., human CD80).

CTLA-4 is a receptor expressed on the surface of T cells (e.g., cytotoxic T cells) and is known to compete with the co-stimulatory molecule CD28 to bind to CD80 and CD86 on antigen-presenting cells, thereby suppressing CD28 co-stimulatory signals and inhibiting T cell activation. The CTLA-4 inhibitor of the present invention inhibits binding of CTLA-4 to CD80, thereby blocking the suppression of co-stimulatory signals and activating T cells (e.g., cytotoxic T cells). Accordingly, the present invention also provides a T cell activating agent (preferably, a cytotoxic T cell activating agent) comprising the cyclic peptide of the present invention described above (hereinafter referred to as the T cell activating agent of the present invention). T cells in a subject (e.g., human) can be activated by administering an effective amount of the cyclic peptide of the present invention.

In addition, it is also known that inhibition of the binding of CTLA-4 to CD80 and CD86 on antigen-presenting cells enhances anti-tumor immunity by blocking suppressive regulation in activated T cells and enhancing proliferation, activation and cytotoxicity or the like of tumor antigen-specific T cells. Accordingly, the CTLA-4 inhibitor of the present invention is useful for enhancing anti-tumor immunity and preventing or treating a tumor. The present invention provides an agent for preventing or treating a tumor/an agent for enhancing anti-tumor immunity comprising the cyclic peptide of the present invention. A tumor in a subject (e.g., human) can be prevented or treated by administering an effective amount of the cyclic peptide of the present invention to the subject. In addition, anti-tumor immunity in a subject (e.g., human) can be enhanced by administering an effective amount of the cyclic peptide of the present invention to the subject. The subject is preferably a human tumor patient. The cyclic peptide of the present invention may be useful for treating a metastatic cancer, suppressing metastasis or suppressing recurrence of a tumor.

As used herein, the term “effective amount” means an amount which results in an aimed effect (e.g., therapeutic effective) on the subject, and means, for example, that in the subject who has received the amount, the symptom of the disease or condition is alleviated, mitigated, eliminated or the development of the symptom of the disease or condition is delayed or inhibited compared with a subject who has not received the amount. An effective amount can be appropriately determined by doctors in view of the age, weight, sex and the severity of the disease or the like of the subject.

As used herein, the term “treatment of a tumor” means bringing about at least one effect of reduction of a tumor size, suppression of tumor growth (retardation or stopping), suppression of tumor metastasis (retardation or stopping), suppression of the recurrence (prevention or retardation), and alleviation of one or a plurality of symptoms associated with cancer.

The type of tumor is not particularly limited, but examples include leukemia (for example, acute myelogenous leukemia, chronic myelogenous leukemia, acute lymphocytic leukemia, chronic lymphocytic leukemia), malignant lymphoma (Hodgkin lymphoma, non-Hodgkin lymphoma (for example, adult T cell leukemia, follicular lymphoma, diffuse large B-cell lymphoma)), multiple myeloma, myelodysplastic syndrome, head or neck cancer, esophageal cancer, esophageal adenocarcinoma, stomach cancer, colorectal cancer, colorectal cancer, rectal cancer, liver cancer (for example, hepatoma), gallbladder/bile duct cancer, biliary cancer, pancreatic cancer, thyroid cancer, lung cancer (for example, non-small cell lung cancer (for example, squamous non-small cell lung cancer, non-squamous non-small cell lung cancer), small cell lung cancer), breast cancer, ovarian cancer (for example, serous ovarian cancer), cervical cancer, endometrial cancer, endometrial cancer, vaginal cancer, vulvar cancer, renal cancer (for example, renal cell cancer), urothelial cancer (for example, bladder cancer, upper urinary tract cancer), prostate cancer, testicular tumor (for example, germ cell tumor), osteosarcoma/soft tissue sarcoma, skin cancer (for example, uveal malignant melanoma, malignant melanoma, Merkel cell carcinoma), glioma, brain tumor (for example, glioblastoma), and pleural mesothelioma and cancer of unknown primary).

The cyclic peptides of the invention can be formulated into a pharmaceutical composition according to the conventional methods (for example, Remington's Pharmaceutical Science, latest edition, Mark Publishing Company, Easton, U.S.A). Pharmaceutical dosage forms include, for example, a liquid (for example injection), a dispersant, a suspension, a tablet, a pill, a particle, a suppository, a powder, a fine granule, a granule, a capsule, a syrup, a troche, an inhalant, an ointment, an eye-drop, a nasal drop, an ear-drop, a gel patch and the like. In the pharmaceutical composition, the cyclic peptide of the present invention may be used directly as an active ingredient, or a pharmaceutically acceptable carrier and/or additive may be contained. For example, a surfactant (PEG, Tween or the like), an excipient, an antioxidant (ascorbic acid or the like), a coloring, a fragrance, a preservative, a stabilizer, a buffer (phosphoric acid, citric acid, other organic acids or the like), a chelating agent (EDTA or the like), a suspending agent, an isotonizing agent, a binder, a disintegrant, a lubricant, a plasticizer, a taste masking agent or the like may be contained. Although they are not particularly limited, the pharmaceutical composition may also contain other carriers as appropriate. Specific examples include light anhydrous silicic acid, lactose, crystalline cellulose, mannitol, starch, carmellose calcium, carmellose sodium, hydroxypropyl cellulose, hydroxypropyl methylcellulose, polyvinyl acetaldiethylaminoacetate, polyvinyl pyrrolidone, gelatin, medium-chain triglyceride, polyoxyethylene hydrogenated castor oil 60, sucrose, carboxymethyl cellulose, cornstarch, inorganic salts and the like. In addition, other small molecular weight polypeptide, a protein such as serum albumin, gelatin and immunoglobulin, an amino acid may be contained. When an aqueous solution for injection is formulated, the cyclic peptide of the present invention is dissolved in, for example, isotonic solution containing saline, glucose or other auxiliary agents. Examples of the auxiliary agent include D-sorbitol, D-mannose, D-mannitol, and sodium chloride, and may be used in combination with suitable solubilizing agents, for example, alcohol (ethanol etc.), polyalcohol (propylene glycol, PEG etc.), non ionic surfactant (polysorbate 80, HCO-50) and the like.

The content of the cyclic peptide of the present invention in the pharmaceutical composition is not particularly limited but is, for example, about 0.00001-100 wt %, 0.0001-99.9% or the like of the whole pharmaceutical composition.

The pharmaceutical composition of the present invention can be administered both orally and parenterally. For example, it is administered to a patient by injection or transdermal administration. It can be administered systemically or topically by, for example, intravenously injection, intramuscular injection, subcutaneous injection or other dosage forms of injection. It may also be injected at or near the treatment site topically or intramuscularly. Examples of the dosage form of transdermal administration include an ointment, gel, a cream, a plaster, a patch and the like, which can be administered systemically or topically.

All references cited in the present specification, including publication, patent document and the like, are hereby incorporated individually and specifically by reference, to the extent that the entireties thereof have been specifically disclosed herein.

The present invention is explained in more detail in the following by referring to Examples, which are not to be construed as limitative.

### Examples

### 1. Method

### (1) Construction of a Gene Library for Ribosome Display Encoding Randomized Cyclic Peptides

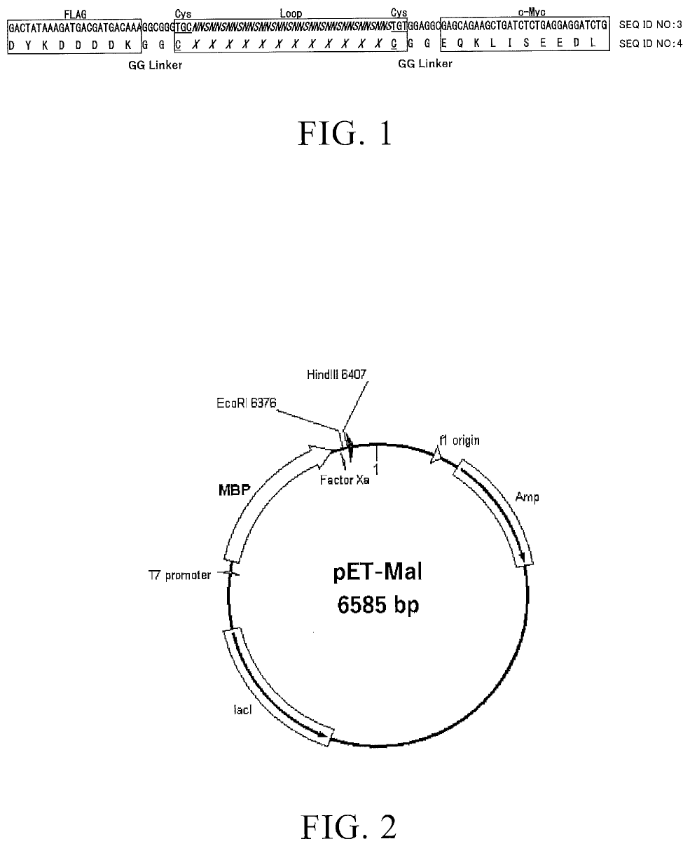

To construct a cyclic peptide library, a library of oligo DNAs was synthesized, in which the oligo DNAs encode randomized amino acid sequences consisting of 12 amino acids flanked by two cysteines, to which a FLAG tag sequence is added to the 5′ side and a c-Myc tag sequence is added to the 3′ side (FIG. 1). The randomized amino acid residues are encoded by 12 NNS codons, wherein N is G, C, T or A and S is G or C.

Furthermore, an oligo DNA of 5′UTR sequence was synthesized, where the 5′UTR sequence comprises T7 promoter and SD sequence necessary for performing ribosome display (the formula below) (FASMAC). FLAG sequence (underlined portion) was added to the 3′ end as a region overlapped with oligo DNAs in the cyclic peptide library.

5′ UTR:
(SEQ ID NO: 5)
GAAATTAATACGACTCACTATAGGGAGACCACAACGGTTTCCCTCTAGAA
ATAATTTTGTTTAACTTTAAGAAGGAGATATACCAATGGACTATAAAGAT
GACGATGACAAA

Using a M13KO7-derived phage genome as a template, a partial sequence of M13 phage genIII (g3p) was amplified by PCR with the primer set below and KOD Plus DNA Polymerase (TOYOBO) (denature: 94° C. for 10 seconds, annealing: 57° C. for 30 seconds, extension: 68° C. for 60 seconds, cycles: 25 cycles), and purified by NeucleoSpin Gel and PCR Clean-up (Takara).

Myc-g3p:
(SEQ ID NO: 6)
GAGCAGAAGCTGATCTCTGAGGAGGATCTGAAGCTTGAATATCAAGGCCA
ATCGTCTGAC
g3p-SecMstop:
(SEQ ID NO: 7)
CTCGAGTTATTCATTAGGTGAGGCGTTGAGGGCCAGCACGGATGCCTTGC
GCCTGGCTTATCCAGACGGGCGTGCTGAATTTTGCGCCGGAAACGTCACC
AATGAAAC

PCR reaction solution (KOD Plus DNA Polymerase, 500 μL total) containing 1 pmol each of the synthesized oligo DNAs (5′UTR, and the oligo DNA library encoding randomized cyclic peptides) and g3p gene fragment was prepared and subjected to PCR for 15 cycles (denature: 94° C. for 10 seconds, annealing: 58° C. for 30 seconds, extension: 68° C. for 60 seconds). The reaction solution (50 μL×10 tubes) was additionally added with two primers below (10 pmol each) and 1 μL of KOD Plus DNA Polymerase and subjected to PCR for 10 cycles (denature: 94° C. for 10 seconds, annealing: 58° C. for 30 seconds, extension: 68° C. for 60 seconds).

5′primer:
(SEQ ID NO: 9)
GAAATTAATACGACTCACTATAGGGAGACCACAACGGTTTCCCTCTAG
SecMstop:
(SEQ ID NO: 8)
GGATTAGTTATTCATTAGGTGAGGCGTTGAGG

The reaction product was separated by electrophoresis using 1% agarose, and the amplified band in which all the intended fragments were connected was cut out and purified by NeucleoSpin Gel and PCR clean-up (Takara) to finally obtain a gene library for ribosome display encoding randomized cyclic peptides.

### (2) Biotinylation of Antigen Protein

CTLA-4 protein (Recombinant Human CTLA4-Fc chimera: R&D systems) was biotinylated according to the standard protocol for EZ-Link NHS-PEO12-Biotin (PIERCE). Biotinylation of CTLA-4 protein was confirmed mobility shift of the band in SDS-PAGE. The concentration of biotinylated CTLA-4 protein was measured by using BCA Protein Assay Kit (PIERCE)

### (3) In Vitro Transcription

1 μg of the purified gene library DNA was transcribed into mRNA with 20 μl of In vitro transcription Kit (T7 Ribomax™ Express Large Scale RNA Production System: Promega), and the obtained mRNA library was column-purified (NucleoSpin RNA clean-up: Takara).

### (4) In Vitro Selection

According to the method of Shimizu et al., a cell-free translation system (PUREsystem) for use in ribosome display was prepared as a reaction solution partially optimized for S—S bond formation (Shimizu et al., (2005) Methods, vol. 36, p. 299-304). Oxidized glutathione (GSSG: SIGMA) and reduced glutathione (GSH: SIGMA) were added at a final concentration of 3 mM each, and a molecular chaperon, DsbC, was added at a final concentration of 0.63 μM. Prepared reaction solution (100 μl) was added with 10 pmol of mRNA library and incubated at 37° C. for 30 min to form ribosome display complexes (peptide-ribosome-mRNA complexes). The reaction product was added with 500 μL of ice-cold wash buffer solution (50 mM Tris-OAc, pH7.5, 150 mM NaCl, 50 mM Mg(OAc)₂, 0.5% Tween 20, 1 μg/mL Saccharomyces cerevisiae total RNA (Sigma)).

Dynabeads MyOne Streptavidin T1 magnetic beads (100 μL slurry, Invitrogen) pre-blocked overnight with 5% SuperBlock at 4° C. was trapped with MagneSphere Magnetic Separation Stand (Promega) and washed twice with 500 μL of wash buffer solution. 100 pmol of biotinylated antigen protein was added and immobilized on the magnetic beads at 4° C. After 60 min, the magnetic beads were trapped with MagneSphere Magnetic Separation Stand (Promega) and washed 3 times with 500 μL of wash buffer solution. The recovered magnetic beads were added with post-translation reaction solution containing ribosome display complexes (peptide-ribosome-mRNA complexes) and stirred with rotation at 4° C. for 1 hr. A supernatant was discarded by MagneSphere Magnetic Separation Stand (Promega), and the recover magnetic beads were added with 1 mL of wash buffer solution and stirred with rotation at 4° C. for 5 min. This series of operations were repeated 30 times, and then the recovered magnetic beads were added with 100 μL of Elution buffer solution (50 mM Tris-HCl, pH7.4, 150 mM NaCl, 50 mM EDTA) and allowed to stand at 4° C. for 10 min to release mRNA from the magnetic beads. The magnetic beads were trapped by MagneSphere Magnetic Separation Stand (Promega) to recover a supernatant containing mRNA, and the mRNA was purified by NucleoSpin RNA clean-up (Takara). A part of purified mRNA was reacted with RNA-direct SYBR Green Realtime PCR Master Mix (TOYOBO) and quantified by using Light Cycler 480 (Roche).

### (5) RT-PCR

The purified mRNA was reverse-transcribed into cDNA by Transcriptor High Fidelity cDNA Synthesis Kit (Roche), and PCR (50 μL total, denature: 94° C. for 15 seconds, annealing: 57° C. for 30 seconds, extension: 68° C. for 60 seconds, 30 cycles) was performed using the obtained cDNA as a template with KOD Plus DNA Polymerase. The used primers are shown below.

<reverse transcription reverse primer>
(SEQ ID NO: 10)
Myc R: CAGATCCTCCTCAGAGATCAGC
<PCR primers>
(SEQ ID NO: 11)
FLAG F: ATGGACTATAAAGATGACGATGACAAAGG
(SEQ ID NO: 10)
Myc R: CAGATCCTCCTCAGAGATCAGC

The reaction solution after PCR was separated by electrophoresis using 2% agarose, and a band with corresponding size was cut out and purified by NeucleoSpin Gel and PCR clean-up (Takara)

### (6) Reconstruction of Genes for Ribosome Display

The reconstruction of genes for ribosome display of the second or later round was performed as follows. PCR reaction solution (200 μL total) containing purified genes after RT-PCR (1 pmol), 5′UTR (1 pmol), g3p gene fragment (1 pmol), 5′ primer (10 pmol), SecMstop (10 pmol) and KOD Plus DNA Polymerase (TOYOBO) was prepared and subjected to PCR for 15 cycles (denature: 94° C. for 15 seconds, annealing: 57° C. for 30 seconds, extension: 68° C. for 60 seconds). The reaction product was separated by electrophoresis using 1% agarose, and a band with all the genes connected was cut out and purified by NeucleoSpin Gel and PCR clean-up (Takara).

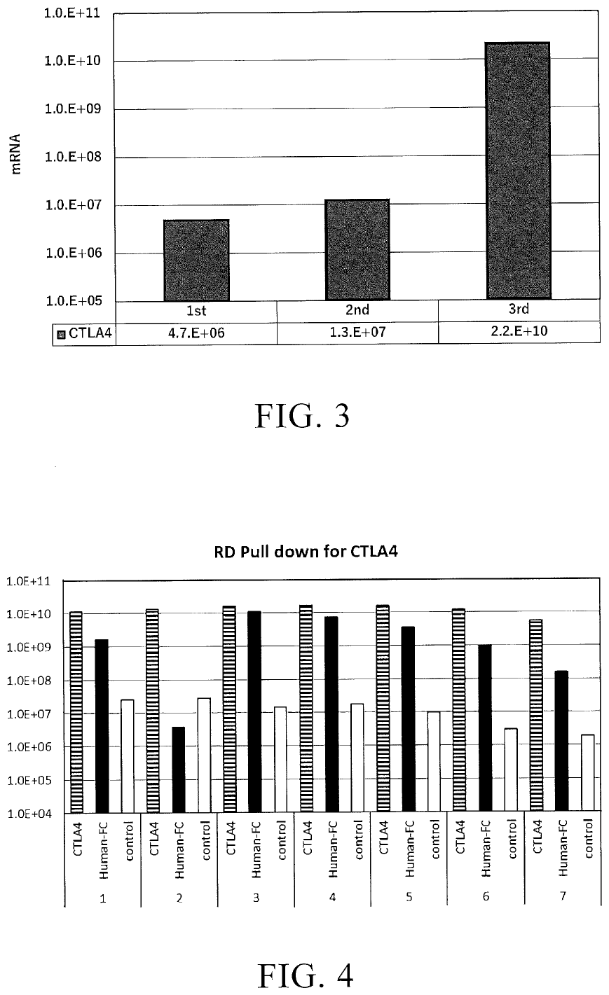

A series of operations including in vitro transcription, in vitro Selection, RT-PCR, and reconstruction of genes for ribosome display was considered as one round, and three rounds were conducted. The amount of recovered mRNA is shown in FIG. 3.

### (7) Subcloning

The recovered genes after three rounds were cloned into E. coli expression vector pET-Mal (FIG. 2) as a N-terminal MBP fusion protein. In brief, PCR reaction solution (KOD Plus DNA Polymerase, 100 μL total) containing the cDNAs obtained by reverse transcription of mRNAs recovered after three rounds,

primer Eco1-(-M)-FLAG_F:
(SEQUENCE ID NO: 12)
CCGAATTCGACTATAAAGATGACGATGACAAAGGC,
and
primerMyc-Hind3_R:
(SEQUENCE ID NO: 13)
AAAAAAAAAAGCTTCAGATCCTCCTCAGAGATC

was prepared and subjected to PCR amplification (denature: 94° C. for 15 seconds, annealing: 57° C. for 30 seconds, extension: 68° C. for 30 seconds) for 20 cycles, and the PCR product was purified by NeucleoSpin Gel and PCR clean-up (Takara). The primer Eco1-(-M)-FLAG_F introduces EcoRI cleavage site for 5′ side and the primer Myc-Hind3_R introduces HindIII cleavage site for 3′ side. 1 μg of the purified PCR product and an expression vector were treated with EcoRI (TOYOBO) and HindIII (TOYOBO) at 37° C. for 1 hour, the reaction product was separated by electrophoresis using 2% agarose, the corresponding bands were cut out and purified by NeucleoSpin Gel and PCR clean-up (Takara). The insert and the vector were mixed at a mol ratio of 5:1 and reacted at room temperature for 30 min by using LigaFast Rapid DNA Ligation Kit (Promega). E. coli BL21 (DE3) competent cells prepared by Z-competent E. coli Transformation Set (ZYMO RESEARCH) were transformed with the ligation product, and cultured on LB agar plates containing ampicillin (final concentration 50 μg/mL) at 37° C., overnight.

### (8) Base Sequence Analysis

94 subcloned E. coli single colonies were subjected to the base sequence analysis. The following sequence primer was used.

(SEQ ID NO: 14)
pET-MALseqF: CCAGAAAGGTGAAATCATGCCGAACATC

### (9) Ribosome Display Pull Down Assay for Enriched Clones

From the clones that were found to be enriched in sequence analysis (Table 3), genes for ribosome display were reconstituted and the ribosome display pull down assay was performed using the same method as for the in vitro selection described above (scaled down to 10 μL) (FIG. 4).

TABLE 3

| Number of | | clone | Sequence | | | | | | | | | | | | | | SEQ |
|---|---|---|---|---|---|---|---|---|---|---|---|---|---|---|---|---|---|
| enriched clones | clone name | No. | Cys | 1 | 2 | 3 | 4 | 5 | 6 | 7 | 8 | 9 | 10 | 11 | 12 | Cys | ID NO: |
| 51 | H12SSI-Ctla4-3-02 | 2 | C | M | H | P | F | L | L | V | V | S | H | H | F | C | 15 |
| 6 | H12SSI-Ctla4-3-51 | 3 | C | G | L | G | Q | G | Y | W | F | K | V | W | F | C | 16 |
| 4 | H12SSI-Ctla4-3-33 | 6 | C | A | K | N | F | G | Y | W | Y | H | Q | W | F | C | 17 |
| 3 | H12SSI-Ctla4-3-80 | 5 | C | G | A | G | K | G | F | W | F | K | V | W | F | C | 18 |
| 2 | H12SSI-Ctla4-2-23 | 1 | C | G | F | H | D | G | F | W | Y | N | V | W | F | C | 19 |
| 2 | H12SSI-Ctla4-3-90 | 4 | C | G | V | N | L | G | F | W | F | N | V | W | F | C | 20 |
| 2 | H12SSI-Ctla4-3-38 | 7 | C | I | I | Q | G | Q | Y | W | W | N | I | W | H | C | 21 |

(10) Overexpression and purification of CTLA-4-binding clone (H12SSI-Ctla4-3-02)

For clones that were found to specifically bind to CTLA-4 in the ribosome display pull down assay, N-terminal MBP fusion proteins were overexpressed in E. coli (BL21BE3) and purified. E. coli transformants were inoculated into 200 mL of 2×YT medium containing ampicillin (final concentration 50 μg/mL), incubated at 37° C. for 3-5 hours (OD600=0.5-0.8), and then added with IPTG (final concentration 0.5 mM) and incubated at 25° C. overnight. The grown E. coli cells were collected by centrifugation, resuspended in 60 mL of lysis buffer (20 mM Tris HCl pH 7.5, 500 mM NaCl), and disrupted by a sonicator (Bioruptor UCD-250). The disrupted cell suspension was centrifuged, the supernatant was collected, filtered through a 0.22 μm filter, and subjected to affinity purification by MBPTrap (GE Healthcare). The purified protein solution was subjected to a buffer exchange into PBS by a dialysis membrane. Purity was confirmed by SDS-PAGE and protein concentration was determined using the BCA Protein Assay Kit (PIERCE).

### (11) Affinity Measurement of CTLA-4-Binding Clone (H12SSI-Ctla4-3-02)

The affinity of the purified CTLA-4 binding clone (H12SSI-Ctla4-3-02) to CTLA-4 was measured using BLItz system (forte Bio). All operations were performed according to the instruction manual of the BLItz system. The biotinylated CTLA-4 protein was immobilized on the SA sensor chip via streptavidin.

### (12) Preparation of Error-Prone Library for Affinity Maturation of CTLA-4 Binding Clone (H12SSI-Ctla4-3-02)

Error-Prone Library was prepared by the method of Zhao et al. (Nat. Biotechnol., (1998) vol. 16, p. 258-261) using H12SSI-Ctla4-3-02, which was confirmed to bind specifically to CTLA-4, as a template. The following PCR primer set was used.

<5′ primer>
(SEQUENCE ID NO: 11)
FLAG F: ATGGACTATAAAGATGACGATGACAAAGG
<3′ primer>
(SEQ ID NO: 10)
Myc R: CAGATCCTCCTCAGAGATCAGC

When the above primer set is used for Error-Prone PCR, mutations are also introduced in the GG linker portion immediately after the FLAG tag sequence and in the GG linker portion immediately before the c-Myc tag sequence (see FIG. 1). For the mutant products obtained by Error-Prone PCR, genes for ribosome display were reconstructed by the same method described above.

### (13) Affinity Maturation of CTLA-4-Binding Clone (H12SSI-Ctla4-3-02)

The basic operation of ribosome display was performed in the same manner as described above. Detailed experimental conditions for Affinity Maturation are indicated below. 2 μL of Nanolink streptavidin magnetic beads (SoluLink) were used as magnetic beads to immobilize the antigen, and 1 mL of CTLA-4 protein solution (1 μM) was used as a wash solution. The amount of biotinylated CTLA-4 immobilized on the magnetic beads was 10 pmol in the 1^{st} round, 2 pmol in the 2^{nd} round, and 1 pmol in the 3^{rd} round, respectively. The washing period was 2 hours for the 1^{st} round, 19 hours for the 2^{nd} round, and 67 hours for the 3^{r}d round. The number of wash cycle was one time (2 hours) in the 1^{st} round, two times (about every 10 hours) in the rd round, and six times (about every 10 hours) in the 3rd round. After each round of RT-PCR, Error-Prone PCR was performed in the same manner as described above.

(14) Evaluation of Binding after Affinity Maturation Selection by ELISA

After each round of Affinity Maturation, the cyclic peptide genes were recovered and subcloned into pET-Mal vector (FIG. 2) in the same manner as described above to perform base sequence analysis. For the 94 single colonies analyzed for sequence, E. coli transformants were inoculated into 100 μL of 2×YT medium containing ampicillin (final concentration 50 μg/mL), incubated at 37° C. for 3-5 hours (OD600=0.5-0.8), then added with IPTG (final concentration 0.5 mM) and incubated at 25° C. overnight. 10 μL of E. coli cultured medium was added with 90 μL of PBS and 40 μL of lysis reagent (20 μL BugBuster Protein Extraction Reagent (Novagen), 20 μL Lysozyme solution 2.5 mg/mL) to lyse E. coli cells for 1 hour at room temperature. The cell lysate was added with 40 μL of 12.5% skim milk solution (TBST) and blocked for 1 hour at room temperature. In parallel, antigen protein was immobilized overnight at 4° C. in 384-well plates at 100 ng/20 μL per well, and each well was washed twice with 100 μL/well of TBST, added with 100 μL/well of 5% skim milk solution (TBST) for blocking for 1 hour at room temperature, and washed twice with 100 μL/well of TBST to obtain antigen-immobilized plates. 20 μL/well of blocking-treated E. coli extract was added to the antigen-immobilized plates and gently stirred at room temperature using a plate mixer. After 1 hour, the plates were washed 5 times with 100 μL/well of TBST, 20 μL/well of anti-FLAG M2-HRP conjugate (1:2000 dilution, Sigma) was added and gently stirred on a plate mixer for 1 hour at room temperature. The plates were washed 5 times with 100 μL/well of TBST, and binding of cyclic peptides to the antigen was detected by 20 μL/well of chromogenic substrate (0.4 mg/mL 3,3′,5,5′-Tetramethyl-benzidine, 0.01% hydrogen peroxide). After 15 minutes of reaction at room temperature, the reaction was stopped with 20 μL/well of 2N HCl, and absorbance at 450 nm was measured using a plate reader (infinite F200: TECAN) (Table 5).

(15) Affinity Measurement of CTLA-4 Binding Clones after Affinity Maturation (EC50 Measurement)

For the top 27 clones with the highest ELISA signals among the clones obtained in the Affinity Maturation selection experiment, N-terminal MBP fusion proteins were overexpressed by E. coli and purified using the same method described above, and the EC50 values were calculated by ELISA.

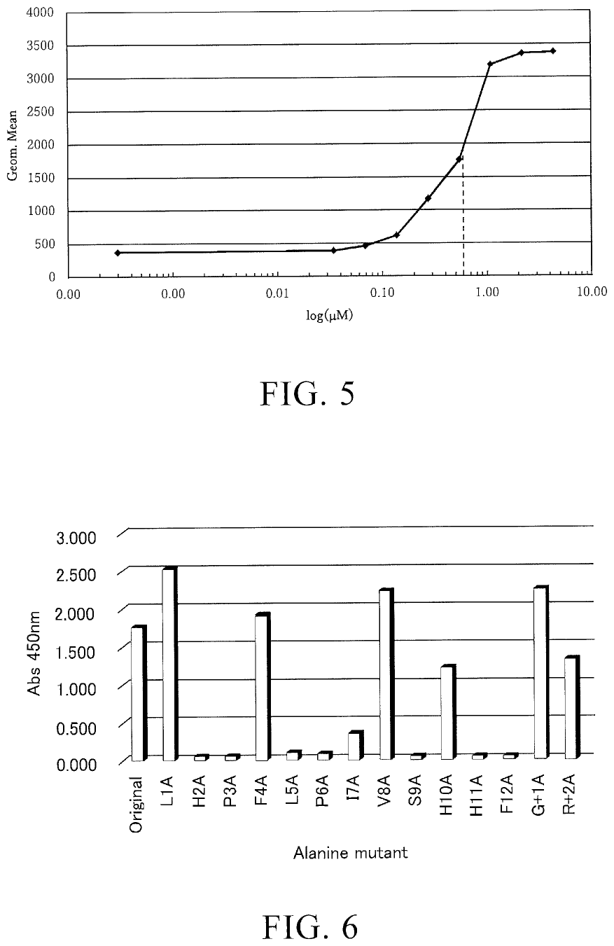

For each of the CTLA-4 binding clones obtained by Affinity Maturation, purified N-terminal MBP fusion protein solution (400 μg/mL) was prepared and samples serially 2-fold diluted with PBS were prepared. CTLA-4 was immobilized overnight at 4° C. in 384-well plates at 100 ng/20 μL per well, and each well was washed twice with 100 μL/well of TBST, added with 100 μL/well of 5% skim milk solution (TBST) for blocking for 1 hour at room temperature, and washed twice with 100 μL/well of TBST to obtain antigen-immobilized plates. 20 μL/well of serially-diluted N-terminal MBP fusion protein solution was added to the antigen-immobilized plates and gently stirred at room temperature using a plate mixer. After 1 hour, the plates were washed 5 times with 100 μL/well of TBST, 20 μL/well of anti-FLAG M2-HRP conjugate (1:2000 dilution, Sigma) was added and gently stirred on a plate mixer for 1 hour at room temperature. Further, the plates were washed 5 times with 100 μL/well of TBST, and binding of N-terminal MBP fusion proteins to the antigen was detected by 20 μL/well of chromogenic substrate (0.4 mg/mL 3,3′,5,5′-Tetramethyl-benzidine, 0.01% hydrogen peroxide). After 15 minutes of reaction at room temperature, the reaction was stopped with 20 μL/well of 2N HCl, and absorbance at 450 nm was measured using a plate reader (infinite F200: TECAN). The graph was plotted with the vertical axis as the absorbance of ELISA and the horizontal axis as the concentration of N-terminal MBP fusion protein of the CTLA4-binding clones (FIG. 5), and the EC50 was calculated from the obtained binding curve (Table 6).

### (16) Measurement of Inhibitory Activity of CTLA-4 Binding Clones (IC50 Measurement)

Among the clones obtained in the Affinity Maturation selection experiment, clone #19 (CMHPFLPIVSHHFCER) (SEQ ID NO: 22), which has high affinity to CTLA-4 in EC50 measurement, was measured for its activity to inhibit binding of CTLA-4 to CD80. The measurement of inhibitory activity was performed using the CTLA4:B7-1[Biotinylated] Inhibitor Screening Assay Kit (BPS Biosciences). The experiment was performed according to the standard protocol attached to the kit.

### (17) Measurement of Affinity and Inhibitory Activity of Synthetic Peptides

For the high-affinity CTLA-4 binding clone #19 (CMHPFLPIVSHHFCER) (SEQ ID NO: 22) obtained by Affinity Maturation and the original clone H12SSI-Ctla4-3-02 (CMHPFLLVVSHHFC) (SEQ ID NO: 15), peptides with PEG4-biotin added via N-terminal lysine residue and cyclized by S—S bond between two cysteine residues (biotinylated clone #19: Biotin-PEG4-KCMHPFLPIVSHHFCER (SEQ ID NO: 24)/biotinylated H12SSI-Ctla4-3-02: Biotin-PEG4-KCMHPFLLVVSHHFC (SEQ ID NO: 23)) were chemically synthesized (Toray Research Center). Affinity for CTLA-4 was measured using the BLItz system (forte Bio). All operations were performed according to the instruction manual of the BLItz system. The biotinylated peptides were immobilized on the SA sensor chip via streptavidin.

In addition, peptides with glycine added to the N-terminus (Gly-clone #19: GCMHPFLPIVSHHFCER (SEQ ID NO: 26)/Gly-H12SSI-Ctla4-3-02: GCMHPFLLVVSHHFC (SEQ ID NO: 25)) were chemically synthesized for the measurement of inhibitory activity. Inhibitory activity was measured using the CTLA4:B7-1 [Biotinylated] Inhibitor Screening Assay Kit (BPS Biosciences) as described above.

### (18) Identification of Amino Acids Essential for CTLA-4 Binding in High Affinity Clones by Alanine Scanning

Using the CTLA-4 binding clone #10 (CLHPFLPIVSHHFCGR) (SEQ ID NO: 27), which had high affinity, as the original clone, a series of oligo DNAs encoding mutants in which any one amino acid residue except cysteine was sequentially replaced with alanine were synthesized and each oligo DNA was subcloned into pET-Mal vector (FIG. 2), and the binding activity of the alanine substitution mutants to CTLA-4 was evaluated by ELISA as described above.

(19) Affinity Maturation with Random Library

Oligo DNAs of a library were synthesized (see below), in which amino acid residues (positions 1, 4, 8, 10, +1 and +2) (corresponding to X₁, X₁, X₈, X₁₀, X₊₁ and X₊₂) other than those essential or advantageous for CTLA-4 binding (2H, 3P, 5L, 6P, 7I, 9S, 11H, 12F) identified by alanine scanning were randomized (NNS).

(No. 19) NNS6:
(SEQ ID NO: 28)
GACTATAAAGATGACGATGACAAAGGCGGTTGCNNSCATCCANNSCTGC
CGATANNSTCANNSCATTTCTGTNNSNNSGGATCCGAGCAGAAGCTGAT
CTCTGAGGAGGATCTG

The gene format for ribosome display (coupling with 5′ UTR and g3p) was prepared in the same manner as described above.

Affinity Maturation was performed in the same manner as described above. The amount of biotinylated CTLA-4 immobilized on the magnetic beads was 10 pmol in the 1^{st} round, 2 pmol in the 2^{nd} round, and 1 pmol in the 3^{nd}-5^{th} rounds, respectively. The washing period was 2 hours for the 1^{st} round, 19 hours for the 2nd round, 67 hours for the 3^{nd} round, 140 hours for the 4^{th} round, and 340 hours for the 5th round. Washing frequency was 1 time (2 hours) in the 1st round, 2 times (every 10 hours) in the 2nd round, 6 times (every 10 hours) in the 3rd round, 14 times (every 10 hours) in the 4th round, and 36 times (every 10 hours) in the 5th round.

### 2. Results and Discussion

### (1) Results of In Vitro Selection and Sequence Analysis

As shown in FIG. 3, sufficient mRNA enrichment was observed in three rounds of selection experiments. The results of sequence analysis (Table 3) indicate that multiple enriched clones were obtained. About half (51/94) of the clones were H12SSI-Ctla4-3-02 (Clone No. 2).

### (2) Ribosome Display Pull Down Assay of Enriched Clones

The CTLA-4 binding activity of the seven clones (Table 3) enriched by in vitro selection was evaluated by Ribosome Display pull down assay (FIG. 4). The results showed that only Clone No. 2 (H12SSI-Ctla4-3-02), which had the highest number in enrichment in the sequence analysis, bound specifically to CTLA-4, while all other clones were specific binders to the Fc moiety of CTLA-4-Fc used as an antigen.

### (3) Large Scale Purification and Affinity Measurement of H12SSI-Ctla4-3-02

H12SSI-Ctla4-3-02, which was confirmed to bind specifically to CTLA-4, was overexpressed in E. coli as an MBP fusion protein, subjected to affinity purification, and the affinity for CTLA-4 was determined by the BLItz system. The results suggest that H12SSI-Ctla4-3-02 has a much lower affinity for CTLA-4 (KD=3.88 μM) than the existing anti-CTLA-4 antibody ipilimumab (KD=5.25±3.62 nM, Assessment Report For Yervoy, European Medicines Agency), as shown in Table 4.

TABLE 4

| kₐ [1/Ms] | k_{d} [1/s] | R² | K_{D} |
|---|---|---|---|
| 3.18E+03 | 1.23E−02 | 0.9932 | 3.88E−06 |

### (4) Affinity Maturation of H12SSI-Ctla4-3-02

An Error-Prone Library was prepared based on H12SSI-Ctla4-3-02, and Affinity Maturation selection experiments were performed. The cyclic peptide genes obtained in each round were subcloned, 94 single colonies were cultured in 96-well plates, and the CTLA-4 binding activity of each MBP-fused cyclic peptide was examined by ELISA. Clones confirmed to have binding activity (S/N ratio not less than 2) by ELISA were summarized in Table 5.

TABLE 5

| | | | ELISA | | | | | | | | | |
|---|---|---|---|---|---|---|---|---|---|---|---|---|
| | | | MBP-fusion | | | GlyGly | | Cys | Loop | | | |
| No. | Round | Sample name | Orencia | SA | S/N(SA) | −2 | −1 | 0 | 1 | 2 | 3 | 4 |
| | | original clone | 0.2278 | 0.0536 | 4.3 | G | G | C | M | H | P | F |
| #1 | 1st | C4-302mat-1-1-24 | 0.7799 | 0.0529 | 14.7 | G | G | C | M | H | P | F |
| #2 | 2nd | C4-302mat-2-100-18 | 0.857 | 0.0533 | 16.1 | G | G | C | M | H | P | F |
| #3 | 3rd | C4-302mat-3-1-48 | 0.9029 | 0.0617 | 14.6 | G | G | C | M | H | P | F |
| #4 | 3rd | C4-302mat-3-100-37 | 1.1667 | 0.0647 | 18.0 | G | G | C | M | H | P | F |
| #5 | 1st | C4-302mat-1-1-35 | 0.5339 | 0.0475 | 11.2 | G | G | C | M | H | P | F |
| #6 | 3rd | C4-302mat-3-1-22 | 1.1053 | 0.0584 | 18.9 | G | G | C | M | H | P | F |
| #7 | 3rd | C4-302mat-3-100-27 | 0.9267 | 0.0559 | 16.6 | G | G | C | M | H | P | F |
| #8 | 2nd | C4-302mat-2-1-2 | 1.2008 | 0.0546 | 23.5 | G | G | C | L | H | P | F |
| #9 | 2nd | C4-302mat-2-1-38 | 0.8075 | 0.0492 | 16.4 | G | G | C | L | H | P | F |
| #10 | 3rd | C4-302mat-3-100-44 | 1.1444 | 0.0617 | 18.5 | G | G | C | L | H | P | F |
| #11 | 3rd | C4-302mat-3-1-45 | 0.6887 | 0.0615 | 11.2 | G | G | C | R | H | P | F |
| #12 | 3rd | C4-302mat-3-100-20 | 0.2214 | 0.0599 | 3.7 | G | G | C | V | H | P | F |
| #13 | 3rd | C4-302mat-3-100-9 | 0.7793 | 0.054 | 14.4 | G | G | C | K | H | P | F |
| #14 | 3rd | C4-302mat-3-1-10 | 0.5861 | 0.0569 | 10.3 | G | G | C | K | H | P | Y |
| #15 | 2nd | C4-302mat-2-1-27 | 0.7848 | 0.0571 | 13.7 | G | G | C | M | H | P | Y |
| #16 | 3rd | C4-302mat-3-100-12 | 1.1792 | 0.0607 | 19.4 | G | G | C | M | H | P | Y |
| #17 | 2nd | C4-302mat-2-1-3 | 0.8018 | 0.0542 | 14.8 | G | G | C | M | H | P | F |
| #18 | 2nd | C4-302mat-2-100-27 | 1.2889 | 0.0512 | 25.2 | G | G | C | M | H | P | F |
| #19 | 3rd | C4-302mat-3-1-27 | 1.0658 | 0.0591 | 18.0 | G | G | C | M | H | P | F |
| #20 | 3rd | C4-302mat-3-1-23 | 1.2104 | 0.0614 | 19.7 | G | G | C | M | H | P | F |
| #21 | 3rd | C4-302mat-3-1-32 | 0.6564 | 0.0578 | 11.4 | G | G | C | M | H | P | F |
| #23 | 3rd | C4-302mat-3-1-31 | 0.868 | 0.0656 | 13.2 | G | S | C | M | H | P | F |
| #24 | 3rd | C4-302mat-3-1-5 | 0.4377 | 0.0739 | 5.9 | G | G | C | M | H | P | F |
| #25 | 3rd | C4-302mat-3-1-12 | 0.9496 | 0.053 | 17.9 | G | G | C | M | H | P | F |
| #22 | 3rd | C4-302mat-3-1-35 | 0.2989 | 0.0569 | 5.3 | G | G | C | M | H | P | F |
| #26 | 2nd | C4-302mat-2-1-35 | 0.9038 | 0.0504 | 17.9 | G | G | C | M | H | P | F |
| #27 | 2nd | C4-302mat-2-100-4 | 0.5451 | 0.0587 | 9.3 | G | G | C | M | H | P | F |
| #28 | 1st | C4-302mat-1-1-21 | 0.1553 | 0.0572 | 2.7 | G | G | C | I | H | P | F |
| #29 | 1st | C4-302mat-1-100-23 | 0.25 | 0.0467 | 5.4 | G | G | C | V | H | P | F |
| #30 | 2nd | C4-302mat-2-1-40 | 0.3661 | 0.0529 | 6.9 | G | G | C | M | H | P | F |
| #31 | 3rd | C4-302mat-3-1-2 | 1.4489 | 0.0662 | 21.9 | G | G | C | M | H | P | Y |
| #32 | 1st | C4-302mat-1-1-37 | 0.6653 | 0.0467 | 14.2 | G | G | C | M | H | P | F |
| #33 | 3rd | C4-302mat-3-1-14 | 0.9848 | 0.0634 | 15.5 | G | G | C | M | H | P | F |
| #34 | 2nd | C4-302mat-2-1-12 | 0.5104 | 0.06 | 8.5 | G | G | C | K | H | P | F |
| #35 | 2nd | C4-302mat-2-1-24 | 0.1402 | 0.0576 | 2.4 | G | G | C | M | H | P | F |
| #36 | 3rd | C4-302mat-3-1-8 | 0.5553 | 0.0674 | 8.2 | G | G | C | M | H | P | F |
| #37 | 2nd | C4-302mat-2-1-37 | 0.1198 | 0.0514 | 2.3 | G | G | C | M | H | P | F |
| #38 | 2nd | C4-302mat-2-100-33 | 0.1165 | 0.0499 | 2.3 | G | S | C | M | H | P | F |
| #39 | 3rd | C4-302mat-3-1-9 | 0.5735 | 0.0653 | 8.8 | G | G | C | M | H | P | Y |
| #40 | 2nd | C4-302mat-2-100-24 | 0.2268 | 0.0566 | 4.0 | G | G | C | M | H | P | Y |
| #41 | 3rd | C4-302mat-3-1-15 | 0.3511 | 0.0655 | 5.4 | G | G | C | M | H | P | Y |
| #42 | 3rd | C4-302mat-3-1-25 | 0.2637 | 0.062 | 4.3 | G | D | C | K | H | P | Y |
| #43 | 2nd | C4-302mat-2-100-40 | 0.2036 | 0.0516 | 3.9 | G | G | C | M | H | P | F |
| #44 | 3rd | C4-302mat-3-1-11 | 0.4123 | 0.0509 | 8.1 | G | G | C | M | H | P | F |
| #45 | 3rd | C4-302mat-3-100-39 | 0.136 | 0.0585 | 2.3 | G | G | C | K | H | P | F |

| | | | | | | | | | | | | | | SEQ |
|---|---|---|---|---|---|---|---|---|---|---|---|---|---|---|
| | | | Loop | | | | | | | | Cys | GlyGly | | ID |
| No. | Round | Sample name | 5 | 6 | 7 | 8 | 9 | 10 | 11 | 12 | G | +1 | +2 | NO: |
| | | original clone | L | L | V | V | S | H | H | F | C | G | G | 29 |
| #1 | 1st | C4-302mat-1-1-24 | L | P | V | V | S | H | H | F | C | G | G | 30 |
| #2 | 2nd | C4-302mat-2-100-18 | L | P | V | V | S | H | H | F | C | R | G | 31 |
| #3 | 3rd | C4-302mat-3-1-48 | L | P | V | V | S | H | H | F | C | G | A | 32 |
| #4 | 3rd | C4-302mat-3-100-37 | L | P | V | V | S | H | H | F | C | G | R | 33 |
| #5 | 1st | C4-302mat-1-1-35 | L | P | V | A | S | H | H | F | C | G | G | 34 |
| #6 | 3rd | C4-302mat-3-1-22 | L | P | V | A | S | H | H | F | C | G | R | 35 |
| #7 | 3rd | C4-302mat-3-100-27 | L | P | V | A | S | H | H | F | C | A | G | 36 |
| #8 | 2nd | C4-302mat-2-1-2 | L | P | V | V | S | H | H | F | C | G | D | 37 |
| #9 | 2nd | C4-302mat-2-1-38 | L | P | L | V | S | H | H | F | C | G | G | 38 |
| #10 | 3rd | C4-302mat-3-100-44 | L | P | I | V | S | H | H | F | C | G | R | 39 |
| #11 | 3rd | C4-302mat-3-1-45 | L | P | I | V | S | R | H | F | C | R | G | 40 |
| #12 | 3rd | C4-302mat-3-100-20 | L | P | V | V | S | H | H | F | C | R | G | 41 |
| #13 | 3rd | C4-302mat-3-100-9 | L | P | V | V | S | H | H | F | C | G | G | 42 |
| #14 | 3rd | C4-302mat-3-1-10 | L | P | V | A | S | H | H | F | C | R | G | 43 |
| #15 | 2nd | C4-302mat-2-1-27 | L | P | V | I | S | H | H | F | C | G | G | 44 |
| #16 | 3rd | C4-302mat-3-100-12 | L | P | V | I | S | H | H | F | C | G | R | 45 |
| #17 | 2nd | C4-302mat-2-1-3 | L | P | V | T | S | H | H | F | C | G | G | 46 |
| #18 | 2nd | C4-302mat-2-100-27 | L | P | I | V | S | H | H | F | C | G | S | 47 |
| #19 | 3rd | C4-302mat-3-1-27 | L | P | I | V | S | H | H | F | C | E | R | 48 |
| #20 | 3rd | C4-302mat-3-1-23 | L | P | I | A | S | L | H | F | C | G | R | 49 |
| #21 | 3rd | C4-302mat-3-1-32 | L | P | I | L | S | H | H | F | C | A | G | 50 |
| #23 | 3rd | C4-302mat-3-1-31 | L | P | V | A | S | R | H | F | C | G | R | 51 |
| #24 | 3rd | C4-302mat-3-1-5 | L | P | V | V | S | R | H | F | C | G | G | 52 |
| #25 | 3rd | C4-302mat-3-1-12 | L | P | V | V | S | R | H | F | C | G | R | 53 |
| #22 | 3rd | C4-302mat-3-1-35 | L | Q | T | V | S | H | H | F | C | G | G | 54 |
| #26 | 2nd | C4-302mat-2-1-35 | L | Q | V | V | S | H | H | F | C | G | S | 55 |
| #27 | 2nd | C4-302mat-2-100-4 | L | Q | V | V | S | H | H | F | C | G | G | 56 |
| #28 | 1st | C4-302mat-1-1-21 | L | Q | V | V | S | H | H | F | C | G | G | 57 |
| #29 | 1st | C4-302mat-1-100-23 | L | Q | V | V | S | H | H | F | C | G | G | 58 |
| #30 | 2nd | C4-302mat-2-1-40 | L | Q | V | A | S | H | H | F | C | G | G | 59 |
| #31 | 3rd | C4-302mat-3-1-2 | L | Q | V | V | S | H | H | F | C | G | R | 60 |
| #32 | 1st | C4-302mat-1-1-37 | L | R | V | V | S | H | H | F | C | G | G | 61 |
| #33 | 3rd | C4-302mat-3-1-14 | L | R | V | V | S | H | H | F | C | G | R | 62 |
| #34 | 2nd | C4-302mat-2-1-12 | L | R | V | V | S | H | H | F | C | R | R | 63 |
| #35 | 2nd | C4-302mat-2-1-24 | L | R | I | V | S | Q | H | F | C | R | R | 64 |
| #36 | 3rd | C4-302mat-3-1-8 | L | K | I | A | S | H | H | F | C | G | G | 65 |
| #37 | 2nd | C4-302mat-2-1-37 | L | L | V | A | S | H | H | F | C | G | V | 66 |
| #38 | 2nd | C4-302mat-2-100-33 | L | L | V | A | S | H | H | F | C | R | G | 67 |
| #39 | 3rd | C4-302mat-3-1-9 | L | L | V | A | S | H | H | F | C | E | R | 68 |
| #40 | 2nd | C4-302mat-2-100-24 | L | L | V | V | S | H | H | F | C | G | R | 69 |
| #41 | 3rd | C4-302mat-3-1-15 | L | L | V | V | S | R | H | F | C | G | R | 70 |
| #42 | 3rd | C4-302mat-3-1-25 | L | L | T | V | S | H | H | F | C | G | R | 71 |
| #43 | 2nd | C4-302mat-2-100-40 | L | L | V | V | S | R | H | F | C | G | R | 72 |
| #44 | 3rd | C4-302mat-3-1-11 | L | L | T | V | S | R | H | F | C | G | G | 73 |
| #45 | 3rd | C4-302mat-3-100-39 | L | L | I | V | S | H | H | F | C | G | R | 74 |

The number of binding clones obtained in each round was 5 in the 1^{st} round, 15 in the 2^{nd} round, and 25 in the 3^{rd} round. It was confirmed that the number of clones with CTLA-4 binding activity increased with increasing selection pressure. Sequence analysis of these clones revealed that the leucine at position 6 (corresponding to X₆) of the loop portion tends to be replaced by proline, glutamine, or arginine. In particular, it was confirmed that the proline mutant tends to have high binding activity.

(5) Evaluation of Affinity after Affinity Maturation (EC50 Measurement)

For clones that showed high S/N ratio in ELISA (L6P mutants (No. #1-#25), L6Q mutant (No. #26) and L6R mutant (No. #32)), MBP fusion cyclic peptides were overexpressed in E. coli, subjected to affinity purification and the EC50 was measured. The results revealed that almost all clones had improved affinity compared to the original clone (Table 6). In particular, the CTLA-4 affinity of clones #19, #10 and #25 was about 50-fold improved over the original clone.

TABLE 6

| | | GlyGly | | Cys | Loop | | | | | | | | | | | | Cys | GlyGly | | EC50 | SEQ |
|---|---|---|---|---|---|---|---|---|---|---|---|---|---|---|---|---|---|---|---|---|---|
| No. | Sample name | −2 | −1 | 0 | 1 | 2 | 3 | 4 | 5 | 6 | 7 | 8 | 9 | 10 | 11 | 12 | 0 | +1 | +2 | (μM) | ID NO: |
| Original | C4-302mat-2-1-5 | G | G | C | M | H | P | F | L | L | V | V | S | H | H | F | C | G | G | 3.921 | 29 |
| #19 | C4-302mat-3-1-27 | G | G | C | M | H | P | F | L | P | I | V | S | H | H | F | C | E | R | 0.075 | 48 |
| #10 | C4-302mat-3-100-44 | G | G | C | L | H | P | F | L | P | I | V | S | H | H | F | C | G | R | 0.082 | 39 |
| #25 | C4-302mat-3-1-12 | G | G | C | M | H | P | F | L | P | V | V | S | R | H | F | C | G | R | 0.085 | 53 |
| #20 | C4-302mat-3-1-23 | G | G | C | M | H | P | F | L | P | I | A | S | L | H | F | C | G | R | 0.160 | 49 |
| #3 | C4-302mat-3-1-48 | G | G | C | M | H | P | F | L | P | V | V | S | H | H | F | C | G | A | 0.187 | 32 |
| #18 | C4-302mat-2-100-27 | G | G | C | M | H | P | F | L | P | I | V | S | H | H | F | C | G | S | 0.189 | 47 |
| #16 | C4-302mat-3-100-12 | G | G | C | M | H | P | Y | L | P | V | I | S | H | H | F | C | G | R | 0.217 | 45 |
| #6 | C4-302mat-3-1-22 | G | G | C | M | H | P | F | L | P | V | A | S | H | H | F | C | G | R | 0.222 | 35 |
| #4 | C4-302mat-3-100-37 | G | G | C | M | H | P | F | L | P | V | V | S | H | H | F | C | G | R | 0.225 | 33 |
| #5 | C4-302mat-1-1-35 | G | G | C | M | H | P | F | L | P | V | A | S | H | H | F | C | G | G | 0.288 | 34 |
| #11 | C4-302mat-3-1-45 | G | G | C | R | H | P | F | L | P | I | V | S | R | H | F | C | R | G | 0.300 | 40 |
| #21 | C4-302mat-3-1-32 | G | G | C | M | H | P | F | L | P | I | L | S | H | H | F | C | A | G | 0.322 | 50 |
| #23 | C4-302mat-3-1-31 | G | S | C | M | H | P | F | L | P | V | A | S | R | H | F | C | G | R | 0.342 | 51 |
| #7 | C4-302mat-3-100-27 | G | G | C | M | H | P | F | L | P | V | A | S | H | H | F | C | A | G | 0.352 | 36 |
| #2 | C4-302mat-2-100-18 | G | G | C | M | H | P | F | L | P | V | V | S | H | H | F | C | R | G | 0.369 | 31 |
| #8 | C4-302mat-2-1-2 | G | G | C | L | H | P | F | L | P | V | V | S | H | H | F | C | G | D | 0.474 | 37 |
| #22 | C4-302mat-3-1-35 | G | G | C | M | H | P | F | L | Q | T | V | S | H | H | F | C | G | G | 0.668 | 54 |
| #24 | C4-302mat-3-1-5 | G | G | C | M | H | P | F | L | P | V | V | S | R | H | F | C | G | G | 0.674 | 52 |
| #14 | C4-302mat-3-1-10 | G | G | C | K | H | P | Y | L | P | V | A | S | H | H | F | C | R | G | 0.703 | 43 |
| #17 | C4-302mat-2-1-3 | G | G | C | M | H | P | F | L | P | V | T | S | H | H | F | C | G | G | 0.826 | 46 |
| #13 | C4-302mat-3-100-9 | G | G | C | K | H | P | F | L | P | V | V | S | H | H | F | C | G | G | 0.832 | 42 |
| #9 | C4-302mat-2-1-38 | G | G | C | L | H | P | F | L | P | L | V | S | H | H | F | C | G | G | 0.867 | 38 |
| #12 | C4-302mat-3-100-20 | G | G | C | V | H | P | F | L | P | V | V | S | H | H | F | C | R | G | 0.889 | 41 |
| #1 | C4-302mat-1-1-24 | G | G | C | M | H | P | F | L | P | V | V | S | H | H | F | C | G | G | 1.035 | 30 |
| #32 | C4-302mat-1-1-37 | G | G | C | M | H | P | F | L | R | V | V | S | H | H | F | C | G | G | 1.493 | 61 |
| #15 | C4-302mat-2-1-27 | G | G | C | M | H | P | Y | L | P | V | I | S | H | H | F | C | G | G | 1.637 | 44 |
| #27 | C4-302mat-2-100-4 | G | G | C | M | H | P | F | L | Q | V | V | S | H | H | F | C | G | G | 4.181 | 56 |

(6) Evaluation of Inhibitory Activity after Affinity Maturation (IC50 Measurement)

For clone #19 which showed high affinity in the EC50 measurement, IC50 of the MBP fusion protein was measured using the CTLA4:B7-1[Biotinylated] Inhibitor Screening Assay Kit (BPS Bioscience). The results are shown in Table 7. The IC50 value of clone #19, whose affinity was increased by Affinity Maturation, was 1.36 μM.

TABLE 7

| Clone | GlyGly | | Cys | Loop | | | | | | | | | | | | Cys | GlyGly | | IC50 | SEQ |
|---|---|---|---|---|---|---|---|---|---|---|---|---|---|---|---|---|---|---|---|---|
| No. | −2 | −1 | 0 | 1 | 2 | 3 | 4 | 5 | 6 | 7 | 8 | 9 | 10 | 11 | 12 | 0 | +1 | +2 | (μM) | ID NO: |
| #19 | G | G | C | M | H | P | F | L | P | I | V | S | H | H | F | C | E | R | 1.36 | 48 |

### (7) Measurement of Affinity and Inhibitory Activity of Synthetic Peptides

From the results shown in Table 8, it was confirmed that the inhibitory activity was maintained in the synthesized peptide (Clone No. #19) (IC50=11.5 μM).

TABLE 8

| Clone | Gly | Cys | Loop | | | | | | | | | | | | Cys | GlyGly | | IC50 | SEQ |
|---|---|---|---|---|---|---|---|---|---|---|---|---|---|---|---|---|---|---|---|
| No. | − 1 | 0 | 1 | 2 | 3 | 4 | 5 | 6 | 7 | 8 | 9 | 10 | 11 | 12 | 0 | +1 | +2 | (μM) | ID NO: |
| #19 | G | C | M | H | P | F | L | P | I | V | S | H | H | F | C | E | R | 11.50 | 26 |

From the results of affinity measurement by the BLItz system (Table 9), it was confirmed that the high binding activity to CTLA-4 protein was maintained even for the synthetic peptide, and that clone #19 had about 3-fold higher affinity than the original clone (H12SSI-Ctla4-3-02).

TABLE 9

| | | | | | | | | | | | | | | | | | | | | | | SEQ |
|---|---|---|---|---|---|---|---|---|---|---|---|---|---|---|---|---|---|---|---|---|---|---|
| Clone | Biotin- | | Loop | | | | | | | | | | | | | | | ka | kd | | | ID |
| No. | Lys | 0 | 1 | 2 | 3 | 4 | 5 | 6 | 7 | 8 | 9 | 10 | 11 | 12 | 0 | +1 | +2 | (1/Ms) | (1/s) | R² | K_{D} | NO: |
| Original | Biotin-K | C | M | H | P | F | L | L | V | V | S | H | H | F | C | | | 1.15E+05 | 2.20E−02 | 0.9782 | 1.91E−07 | 23 |
| #19 | Biotin-K | C | M | H | P | F | L | P | I | V | S | H | H | F | C | E | R | 2.08E+05 | 1.28E−02 | 0.9969 | 6.18E−08 | 24 |

### (8) Measurement of Affinity of Synthetic Peptide for CTLA-4 Expressed on the Cell Surface

FACS analysis on the cells (CHO cells) expressing CTLA-4 showed that the EC50 of No. #19 was 0.47 μM, confirming that it binds specifically to CTLA-4 with its natural structure expressed on the cell surface (FIG. 5).

### (9) Identification of Amino Acids Essential for CTLA-4 Binding by Canine Scanning

Alanine scanning was performed on the high-affinity clone No. #10 (CLHPFLPIVSHHFCGR) (SEQ ID NO: 27) to identify amino acids essential for binding to CTLA-4 (FIG. 6). The results showed that the amino acid residues critical for binding to CTLA-4 were histidine at position 2, proline at position 3, leucine at position 5, proline at position 6, serine at position 9, histidine at position 11, and phenylalanine at position 12, with the N-terminal amino acid of the loop portion as position 1. In addition, since the I7A mutant showed weak binding activity, isoleucine at position 7 was expected to be the residue that slightly affected binding activity.

### (10) Affinity Maturation by Random Library and Results of Sequence Analysis

The results of sequence of clones that showed CTLA-4 binding activity in ELISA after Affinity Maturation from a random library are shown in Table 10. Table 11 also summarizes the appearance frequency of amino acids in them.

TABLE 10

| | | | | | | Loop | | | | | | |
|---|---|---|---|---|---|---|---|---|---|---|---|---|
| | | ELISA | GlyGly | | Cys | (NNS) | H | P | (NNS) | L | P | I |
| round | Sample Name | S/N(SA) | −2 | −1 | 0 | 1 | 2 | 3 | 4 | 5 | 6 | 7 |
| 5th | C4m#19-5th4w-NN6-67 | 45.4 | G | G | C | L | H | P | F | L | P | I |
| 5th | C4m#19-5th4w-NN6-15 | 39.9 | G | G | C | L | H | P | F | L | P | I |
| 5th | C4m#19-5th4w-NN6-25 | 38.9 | G | G | C | L | H | P | V | L | P | I |
| 5th | C4m#19-5th4w-NN6-21 | 37.5 | G | G | C | W | H | P | W | L | P | I |
| 5th | C4m#19-5th4w-NN6-39 | 35.6 | G | G | C | W | H | P | F | L | P | I |
| 5th | C4m#19-5th4w-NN6-70 | 35.5 | G | G | C | M | H | P | F | L | P | I |
| 5th | C4m#19-5th2w-NNS6-14 | 34.7 | G | G | C | W | H | P | F | L | P | I |
| 5th | C4m#19-5th4w-NN6-47 | 34.2 | G | G | C | P | H | P | W | L | P | I |
| 5th | C4m#19-5th4w-NN6-54 | 33.3 | G | G | C | L | H | P | G | L | P | I |
| 5th | C4m#19-5th4w-NN6-81 | 32.3 | G | G | C | F | H | P | Y | L | P | I |
| 5th | C4m#19-5th4w-NN6-53 | 32.2 | G | G | C | R | H | P | F | L | P | I |
| 5th | C4m#19-5th4w-NN6-34 | 32.2 | G | G | C | F | H | P | F | L | P | I |
| 5th | C4m#19-5th4w-NN6-4 | 31.6 | G | G | C | R | H | P | Y | L | P | I |
| 5th | C4m#19-5th2w-NNS6-21 | 31.1 | G | G | C | W | H | P | F | L | P | I |
| 5th | C4m#19-5th4w-NN6-20 | 30.6 | G | G | C | S | H | P | Q | L | P | I |
| 5th | C4m#19-5th4w-NN6-77 | 30.5 | G | G | C | R | H | P | F | L | P | I |
| 5th | C4m#19-5th4w-NN6-23 | 30.2 | G | G | C | R | H | P | H | L | P | I |
| 5th | C4m#19-5th4w-NN6-44 | 30.1 | G | G | C | R | H | P | F | L | P | I |
| 5th | C4m#19-5th4w-NN6-92-1 | 30.0 | G | G | C | V | H | P | F | L | P | I |
| 5th | C4m#19-5th4w-NN6-91 | 30.0 | G | G | C | M | H | P | Y | L | P | I |
| 5th | C4m#19-5th4w-NN6-19 | 29.7 | G | G | C | R | H | P | Y | L | P | I |
| 5th | C4m#19-5th4w-NN6-65-1 | 29.5 | G | G | C | W | H | P | F | L | P | I |
| 5th | C4m#19-5th4w-NN6-55-4 | 29.5 | G | G | C | V | H | P | S | L | P | I |
| 5th | C4m#19-5th2w-NNS6-22 | 29.3 | G | G | C | M | H | P | F | L | P | I |
| 5th | C4m#19-5th4w-NN6-93 | 28.9 | G | G | C | C | H | P | W | L | P | I |
| 5th | C4m#19-5th4w-NN6-9 | 28.8 | G | G | C | F | H | P | Y | L | P | I |
| 5th | C4m#19-5th4w-NN6-52 | 28.6 | G | G | C | L | H | P | F | L | P | I |
| 5th | C4m#19-5th2w-NNS6-55 | 28.3 | G | G | C | M | H | P | Y | L | P | I |
| 5th | C4m#19-5th4w-NN6-82 | 28.3 | G | G | C | F | H | P | F | L | P | I |
| 5th | C4m#19-5th4w-NN6-50 | 28.1 | G | G | C | K | H | P | V | L | P | I |
| 5th | C4m#19-5th4w-NN6-78 | 27.9 | G | G | C | W | H | P | A | L | P | I |
| 5th | C4m#19-5th4w-NN6-57 | 27.7 | G | G | C | M | H | P | Y | L | P | I |
| 5th | C4m#19-5th4w-NN6-59 | 27.6 | G | G | C | L | H | P | H | L | P | I |
| 5th | C4m#19-5th4w-NN6-24 | 27.6 | G | G | C | W | H | P | W | L | P | I |
| 5th | C4m#19-5th4w-NN6-32 | 27.2 | G | G | C | P | H | P | F | L | P | I |
| 5th | C4m#19-5th4w-NN6-56 | 27.1 | G | G | C | W | H | P | F | L | P | I |
| 5th | C4m#19-5th2w-NNS6-67 | 25.5 | G | G | C | Q | H | P | W | L | P | I |
| 5th | C4m#19-5th4w-NN6-69-1 | 24.9 | G | G | C | S | H | P | F | L | P | I |
| 5th | C4m#19-5th4w-NN6-69-3 | 24.9 | G | G | C | L | H | P | W | L | P | I |
| 5th | C4m#19-5th4w-NN6-79 | 24.9 | G | G | C | W | H | P | V | L | P | I |
| 5th | C4m#19-5th4w-NN6-68 | 24.7 | G | G | C | L | H | P | W | L | P | I |
| 4th | C4m3127-4th-NNS6-28 | 23.2 | G | G | C | L | H | P | F | L | P | I |
| 5th | C4m#19-5th4w-NN6-7 | 23.0 | G | G | C | R | H | P | F | L | P | I |
| 5th | C4m#19-5th4w-NN6-85 | 22.9 | G | G | C | W | H | P | F | L | P | I |
| 5th | C4n#19-5th2w-NNS6-71 | 22.9 | G | G | C | R | H | P | Y | L | P | I |
| 4th | C4m3127-4th-NNS6-90 | 22.9 | G | G | C | K | H | P | V | L | P | I |
| 5th | C4m#19-5th4w-NN6-37 | 22.7 | G | G | C | L | H | P | F | L | P | I |
| 5th | C4m#19-5th2w-NNS6-94 | 22.4 | G | G | C | F | H | P | Y | L | P | I |
| 5th | C4m#19-5th4w-NN6-51 | 22.1 | G | G | C | W | H | P | Y | L | P | I |
| 5th | C4m#19-5th4w-NN6-11 | 22.1 | G | G | C | R | H | P | F | L | P | I |
| 5th | C4m#19-5th4w-NN6-87 | 21.4 | G | G | C | L | H | P | V | L | P | I |
| 4th | C4m3127-4th-NNS6-87 | 21.4 | G | G | C | R | H | P | W | L | P | I |
| 5th | C4m#19-5th4w-NN6-90 | 21.2 | G | G | C | R | H | P | W | L | P | I |
| 5th | C4m#19-5th2w-NNS6-25 | 21.1 | G | G | C | K | H | P | F | L | P | I |
| 4th | C4m3127-4th-NNS6-83 | 20.7 | G | G | C | R | H | P | F | L | P | I |
| 4th | C4m3127-4th-NNS6-17 | 20.4 | G | G | C | R | H | P | V | L | P | I |
| 5th | C4m#19-5th2w-NNS6-12 | 20.4 | G | G | C | W | H | P | Y | L | P | I |
| 4th | C4m3127-4th-NNS6-72 | 20.0 | G | G | C | V | H | P | Y | L | P | I |
| 5th | C4m#19-5th2w-NNS6-90 | 19.7 | G | G | C | R | H | P | F | L | P | I |
| 5th | C4m#19-5th4w-NN6-86 | 19.7 | G | G | C | A | H | P | V | L | P | I |
| 5th | C4m#19-5th4w-NN6-58 | 18.7 | G | G | C | R | H | P | F | L | P | I |
| 5th | C4m#19-5th2w-NNS6-3 | 18.4 | G | G | C | A | H | P | F | L | P | I |
| 5th | C4m#19-5th4w-NN6-2 | 18.4 | G | G | C | W | H | P | H | L | P | I |
| 5th | C4m#19-5th4w-NN6-71 | 17.4 | G | G | C | P | H | P | W | L | P | I |
| 5th | C4m#19-5th4w-NN6-62 | 17.2 | G | G | C | Q | H | P | Y | L | P | I |
| 5th | C4m#19-5th4w-NN6-30 | 16.7 | G | G | C | W | H | P | F | L | P | I |
| 5th | C4m#19-5th4w-NN6-38 | 16.6 | G | G | C | S | H | P | F | L | P | I |
| 5th | C4m#19-5th2w-NNS6-8 | 16.5 | G | G | C | R | H | P | F | L | P | I |
| 4th | C4m3127-4th-NNS6-16 | 16.5 | G | G | C | W | H | P | Y | L | P | I |
| 5th | C4m#19-5th4w-NN6-63 | 16.5 | G | G | C | W | H | P | Y | L | P | I |
| 5th | C4m#19-5th4w-NN6-14 | 16.1 | G | G | C | W | H | P | F | L | P | I |
| 5th | C4m#19-5th4w-NN6-1 | 16.1 | G | G | C | R | H | P | Y | L | P | I |
| 5th | C4m#19-5th4w-NN6-6 | 16.1 | G | G | C | M | H | P | F | L | P | I |
| 5th | C4m#19-5th2w-NNS6-51 | 15.8 | G | G | C | R | H | P | F | L | P | I |
| 5th | C4m#19-5th4w-NN6-45 | 15.8 | G | G | C | R | H | P | W | L | P | I |
| 4th | C4m3127-4th-NNS6-12 | 15.8 | G | G | C | S | H | P | F | L | P | I |
| 5th | C4m#19-5th2w-NNS6-75 | 15.3 | G | G | C | W | H | P | W | L | P | I |
| 5th | C4m#19-5th4w-NN6-74 | 15.1 | G | G | C | F | H | P | F | L | P | I |
| 5th | C4m#19-5th4w-NN6-5-1 | 14.7 | G | G | C | W | H | P | Y | L | P | I |
| 5th | C4m#19-6th4w-NN6-5-3 | 14.7 | G | G | C | W | H | P | F | L | P | I |
| 5th | C4m#19-5th4w-NN6-46-1 | 14.2 | G | G | C | V | H | P | A | L | P | I |
| 5th | C4m#19-5th4w-NN6-64 | 13.7 | G | G | C | W | H | P | V | L | P | I |
| 4th | C4m3127-4th-NNS6-48 | 13.5 | G | G | C | F | H | P | W | L | P | I |
| 4th | C4m3127-4th-NNS6-64 | 12.9 | G | G | C | L | H | P | Y | L | P | I |
| 4th | C4m3127-4th-NNS6-80 | 12.8 | G | G | C | G | H | P | F | L | P | I |
| 4th | C4m3127-4th-NNS6-79 | 12.8 | G | G | C | L | H | P | F | L | P | I |
| 4th | C4m3127-4th-NNS6-2 | 12.8 | G | G | C | W | H | P | S | L | P | I |
| 5th | C4m#19-5th4w-NN6-65 | 12.5 | G | G | C | W | H | P | F | L | P | I |
| 5th | C4m#19-5th4w-NN6-83 | 12.0 | G | G | C | L | H | P | L | L | P | I |
| 4th | C4m3127-4th-NNS6-60 | 11.9 | G | G | C | W | H | P | F | L | P | I |
| 5th | C4m#19-5th2w-NNS6-53 | 11.4 | G | G | C | L | H | P | W | L | P | I |
| 5th | C4m#19-5th2w-NNS6-45 | 11.0 | G | G | C | R | H | P | F | L | P | I |
| 4th | C4m3127-4th-NNS6-3 | 10.9 | G | G | C | S | H | P | F | L | P | I |
| 5th | C4m#19-5th2w-NNS6-6 | 10.3 | G | G | C | W | H | P | Y | L | P | I |
| 5th | C4m#19-5th2w-NNS6-27 | 9.9 | G | G | C | F | H | P | F | L | P | I |
| 5th | C4m#19-5th2w-NNS6-37 | 9.4 | G | G | C | I | H | P | F | L | P | I |
| 5th | C4m#19-5th2w-NNS6-33 | 9.4 | G | G | C | R | H | P | F | L | P | I |
| 5th | C4m#19-5th2w-NNS6-79 | 9.2 | G | G | C | R | H | P | F | L | P | I |
| 4th | C4m3127-4th-NNS6-85 | 9.1 | G | G | C | G | H | P | F | L | P | I |
| 5th | C4m#19-5th2w-NNS6-30 | 9.0 | G | G | C | W | H | P | F | L | P | I |
| 5th | C4m#19-5th4w-NN6-89 | 8.3 | G | G | C | M | H | P | F | L | P | I |
| 5th | C4m#19-5th2w-NNS6-86 | 7.7 | G | G | C | F | H | P | Y | L | P | I |
| 4th | C4m3127-4th-NNS6-30 | 7.5 | G | G | C | W | H | P | C | L | P | I |
| 4th | C4m3127-4th-NNS6-26 | 7.1 | G | G | C | A | H | P | A | L | P | I |
| 5th | C4m#19-5th2w-NNS6-74 | 7.0 | G | G | C | M | H | P | F | L | P | I |
| 4th | C4m3127-4th-NNS6-89 | 6.6 | G | G | C | W | H | P | F | L | P | I |
| 5th | C4m#19-5th2w-NNS6-84 | 6.4 | G | G | C | W | H | P | F | L | P | I |
| 5th | C4m#19-5th2w-NNS6-28 | 6.3 | G | G | C | A | H | P | Y | L | P | I |
| 5th | C4m#19-5th2w-NNS6-4 | 6.3 | G | G | C | F | H | P | W | L | P | I |
| 5th | C4m#19-5th2w-NNS6-46 | 6.1 | G | G | C | F | H | P | F | L | P | I |
| 5th | C4m#19-5th4w-NN6-61 | 5.9 | G | G | C | P | H | P | M | L | P | I |
| 4th | C4m3127-4th-NNS6-94 | 5.9 | G | G | C | R | H | P | V | L | P | I |
| 5th | C4m#19-5th2w-NNS6-26 | 5.8 | G | G | C | L | H | P | F | L | P | I |
| 5th | C4m#19-5th4w-NN6-36 | 5.7 | G | G | C | R | H | P | Y | L | P | I |
| 5th | C4m#19-5th4w-NN6-75 | 5.6 | G | G | C | W | H | P | F | L | P | I |
| 5th | C4m#19-5th2w-NNS6-70 | 5.1 | G | G | C | R | H | P | F | L | P | I |
| 5th | C4m#19-5th2w-NNS6-34 | 5.0 | G | G | C | C | H | P | F | L | P | I |
| 5th | C4m#19-5th4w-NN6-94 | 5.0 | G | G | C | L | H | P | F | L | P | I |
| 4th | C4m3127-4th-NNS6-66 | 4.8 | G | G | C | A | H | P | H | L | P | I |
| 5th | C4m#19-5th2w-NNS6-43 | 4.8 | G | G | C | R | H | P | F | L | P | I |
| 4th | C4m3127-4th-NNS6-7 | 4.7 | G | G | C | R | H | P | F | L | P | I |
| 5th | C4m#19-5th4w-NN6-72 | 4.6 | G | G | C | W | H | P | W | L | P | I |
| 4th | C4m3127-4th-NNS6-70 | 4.6 | G | G | C | S | H | P | Y | L | P | I |
| 5th | C4m#19-5th2w-NNS6-68 | 4.6 | G | G | C | L | H | P | W | L | P | I |
| 5th | C4m#19-5th2w-NNS6-87 | 4.5 | G | G | C | L | H | P | Y | L | P | I |
| 5th | C4m#19-5th2w-NNS6-66 | 4.4 | G | G | C | R | H | P | F | L | P | I |
| 4th | C4m3127-4th-NNS6-20 | 4.4 | G | G | C | L | H | P | F | L | P | I |
| 5th | C4m#19-5th2w-NNS6-61 | 4.3 | G | G | C | C | H | P | W | L | P | I |
| 4th | C4m3127-4th-NNS6-19 | 4.1 | G | G | C | M | H | P | S | L | P | I |
| 5th | C4m#19-5th4w-NN6-40 | 4.0 | G | G | C | F | H | P | F | L | P | I |
| 5th | C4m#19-5th4w-NN6-49 | 4.0 | G | G | C | M | H | P | Y | L | P | I |
| 5th | C4m#19-5th4w-NN6-80 | 3.8 | G | G | C | R | H | P | F | L | P | I |
| 5th | C4m#19-5th2w-NNS6-80 | 3.6 | G | G | C | L | H | P | F | L | P | I |
| 4th | C4m3127-4th-NNS6-37 | 3.5 | G | G | C | W | H | P | V | L | P | I |
| 5th | C4m#19-5th2w-NNS6-93 | 3.4 | G | G | C | F | H | P | F | L | P | I |
| 4th | C4m3127-4th-NNS6-95 | 3.4 | G | G | C | W | H | P | V | L | P | I |
| 5th | C4m#19-5th2w-NNS6-41 | 3.1 | G | G | C | W | H | P | V | L | P | I |

| | | Loop | | | | | | | | |
|---|---|---|---|---|---|---|---|---|---|---|
| | | (NNS) | S | (NNS) | H | F | Cys | (NNS) | (NNS) | SEQ |
| round | Sample Name | 8 | 9 | 10 | 11 | 12 | 0 | +1 | +2 | ID NO: |
| 5th | C4m#19-5th4w-NN6-67 | R | S | V | H | F | C | P | R | 75 |
| 5th | C4m#19-5th4w-NN6-15 | R | S | V | H | F | C | P | F | 76 |
| 5th | C4m#19-5th4w-NN6-25 | R | S | R | H | F | C | P | V | 77 |
| 5th | C4m#19-5th4w-NN6-21 | L | S | P | H | F | C | P | R | 78 |
| 5th | C4m#19-5th4w-NN6-39 | R | S | L | H | F | C | P | R | 79 |
| 5th | C4m#19-5th4w-NN6-70 | R | S | M | H | F | C | P | L | 80 |
| 5th | C4m#19-5th2w-NNS6-14 | R | S | Y | H | F | C | P | W | 81 |
| 5th | C4m#19-5th4w-NN6-47 | F | S | R | H | F | C | P | V | 82 |
| 5th | C4m#19-5th4w-NN6-54 | R | S | L | H | F | C | P | R | 83 |
| 5th | C4m#19-5th4w-NN6-81 | R | S | W | H | F | C | S | R | 84 |
| 5th | C4m#19-5th4w-NN6-53 | Y | S | V | H | F | C | A | C | 85 |
| 5th | C4m#19-5th4w-NN6-34 | R | S | D | H | F | C | S | R | 86 |
| 5th | C4m#19-5th4w-NN6-4 | V | S | R | H | F | C | P | F | 87 |
| 5th | C4m#19-5th2w-NNS6-21 | R | S | S | H | F | C | P | S | 88 |
| 5th | C4m#19-5th4w-NN6-20 | L | S | D | H | F | C | S | A | 89 |
| 5th | C4m#19-5th4w-NN6-77 | V | S | T | H | F | C | S | R | 90 |
| 5th | C4m#19-5th4w-NN6-23 | W | S | W | H | F | C | G | R | 91 |
| 5th | C4m#19-5th4w-NN6-44 | V | S | W | H | F | C | G | R | 92 |
| 5th | C4m#19-5th4w-NN6-92-1 | R | S | V | H | F | C | A | L | 93 |
| 5th | C4m#19-5th4w-NN6-91 | I | S | P | H | F | C | R | G | 94 |
| 5th | C4m#19-5th4w-NN6-19 | W | S | I | H | F | C | Q | K | 95 |
| 5th | C4m#19-5th4w-NN6-65-1 | S | S | P | H | F | C | G | R | 96 |
| 5th | C4m#19-5th4w-NN6-55-4 | L | S | V | H | F | C | P | R | 97 |
| 5th | C4m#19-5th2w-NNS6-22 | Q | S | V | H | F | C | S | R | 98 |
| 5th | C4m#19-5th4w-NN6-93 | V | S | T | H | F | C | G | L | 99 |
| 5th | C4m#19-5th4w-NN6-9 | R | S | W | H | F | C | G | R | 100 |
| 5th | C4m#19-5th4w-NN6-52 | R | S | V | H | F | C | G | C | 101 |
| 5th | C4m#19-5th2w-NNS6-55 | K | S | V | H | F | C | P | L | 102 |
| 5th | C4m#19-5th4w-NN6-82 | R | 5 | L | H | F | C | C | R | 103 |
| 5th | C4m#19-5th4w-NN6-50 | W | S | W | H | F | C | R | I | 104 |
| 5th | C4m#19-5th4w-NN6-78 | R | S | W | H | F | C | P | R | 105 |
| 5th | C4m#19-5th4w-NN6-57 | V | S | V | H | F | C | P | C | 106 |
| 5th | C4m#19-5th4w-NN6-59 | W | S | L | H | F | C | P | L | 107 |
| 5th | C4m#19-5th4w-NN6-24 | Y | S | H | H | F | C | P | I | 108 |
| 5th | C4m#19-5th4w-NN6-32 | W | S | V | H | F | C | P | F | 109 |
| 5th | C4m#19-5th4w-NN6-56 | Q | S | L | H | F | C | G | V | 110 |
| 5th | C4m#19-5th2w-NNS6-67 | S | S | W | H | F | C | P | R | 111 |
| 5th | C4m#19-5th4w-NN6-69-1 | L | S | V | H | F | C | P | F | 112 |
| 5th | C4m#19-5th4w-NN6-69-3 | V | S | V | H | F | C | A | R | 113 |
| 5th | C4m#19-5th4w-NN6-79 | R | S | C | H | F | C | A | V | 114 |
| 5th | C4m#19-5th4w-NN6-68 | R | S | R | H | F | C | S | R | 115 |
| 4th | C4m3127-4th-NNS6-28 | F | S | S | H | F | C | A | L | 116 |
| 5th | C4m#19-5th4w-NN6-7 | W | S | W | H | F | C | P | F | 117 |
| 5th | C4m#19-5th4w-NN6-85 | F | S | V | H | F | C | G | C | 118 |
| 5th | C4n#19-5th2w-NNS6-71 | A | S | P | H | F | C | P | G | 119 |
| 4th | C4m3127-4th-NNS6-90 | V | S | L | H | F | C | P | W | 120 |
| 5th | C4m#19-5th4w-NN6-37 | S | S | W | H | F | C | G | R | 121 |
| 5th | C4m#19-5th2w-NNS6-94 | S | S | V | H | F | C | G | R | 122 |
| 5th | C4m#19-5th4w-NN6-51 | V | S | W | H | F | C | S | R | 123 |
| 5th | C4m#19-5th4w-NN6-11 | W | S | H | H | F | C | V | C | 124 |
| 5th | C4m#19-5th4w-NN6-87 | R | S | F | H | F | C | W | R | 125 |
| 4th | C4m3127-4th-NNS6-87 | V | S | W | H | F | C | S | R | 126 |
| 5th | C4m#19-5th4w-NN6-90 | V | S | Y | H | F | C | S | S | 127 |
| 5th | C4m#19-5th2w-NNS6-25 | S | S | H | H | F | C | P | Y | 128 |
| 4th | C4m3127-4th-NNS6-83 | F | S | K | H | F | C | E | L | 129 |
| 4th | C4m3127-4th-NNS6-17 | V | S | L | H | F | C | P | G | 130 |
| 5th | C4m#19-5th2w-NNS6-12 | R | S | V | H | F | C | G | R | 131 |
| 4th | C4m3127-4th-NNS6-72 | S | S | I | H | F | C | S | M | 132 |
| 5th | C4m#19-5th2w-NNS6-90 | V | S | F | H | F | C | P | F | 133 |
| 5th | C4m#19-5th4w-NN6-86 | R | S | L | H | F | C | P | H | 134 |
| 5th | C4m#19-5th4w-NN6-58 | S | S | I | H | F | C | C | I | 135 |
| 5th | C4m#19-5th2w-NNS6-3 | F | S | P | H | F | C | P | F | 136 |
| 5th | C4m#19-5th4w-NN6-2 | V | S | S | H | F | C | L | F | 137 |
| 5th | C4m#19-5th4w-NN6-71 | A | S | V | H | F | C | P | F | 138 |
| 5th | C4m#19-5th4w-NN6-62 | W | S | F | H | F | C | P | F | 139 |
| 5th | C4m#19-5th4w-NN6-30 | M | S | F | H | F | C | P | Y | 140 |
| 5th | C4m#19-5th4w-NN6-38 | F | S | L | H | F | C | P | V | 141 |
| 5th | C4m#19-5th2w-NNS6-8 | F | S | L | H | F | C | A | R | 142 |
| 4th | C4m3127-4th-NNS6-16 | F | S | V | H | F | C | P | R | 143 |
| 5th | C4m#19-5th4w-NN6-63 | L | S | R | H | F | C | Q | A | 144 |
| 5th | C4m#19-5th4w-NN6-14 | R | S | V | H | F | C | C | R | 145 |
| 5th | C4m#19-5th4w-NN6-1 | F | S | A | H | F | C | R | S | 146 |
| 5th | C4m#19-5th4w-NN6-6 | K | S | S | H | F | C | R | K | 147 |
| 5th | C4m#19-5th2w-NNS6-51 | V | S | L | H | F | C | P | F | 148 |
| 5th | C4m#19-5th4w-NN6-45 | W | S | V | H | F | C | G | V | 149 |
| 4th | C4m3127-4th-NNS6-12 | R | S | R | H | F | C | P | F | 150 |
| 5th | C4m#19-5th2w-NNS6-75 | K | S | P | H | F | C | A | L | 151 |
| 5th | C4m#19-5th4w-NN6-74 | Y | S | W | H | F | C | A | R | 152 |
| 5th | C4m#19-5th4w-NN6-5-1 | Q | S | L | H | F | C | P | F | 153 |
| 5th | C4m#19-6th4w-NN6-5-3 | L | S | L | H | F | C | G | W | 154 |
| 5th | C4m#19-5th4w-NN6-46-1 | W | S | F | H | F | C | P | S | 155 |
| 5th | C4m#19-5th4w-NN6-64 | R | S | V | H | F | C | C | R | 156 |
| 4th | C4m3127-4th-NNS6-48 | A | S | W | H | F | C | G | R | 157 |
| 4th | C4m3127-4th-NNS6-64 | V | S | S | H | F | C | P | F | 158 |
| 4th | C4m3127-4th-NNS6-80 | F | S | L | H | F | C | P | R | 159 |
| 4th | C4m3127-4th-NNS6-79 | R | S | V | H | F | C | L | R | 160 |
| 4th | C4m3127-4th-NNS6-2 | Y | S | P | H | F | C | P | L | 161 |
| 5th | C4m#19-5th4w-NN6-65 | L | S | L | H | F | C | S | R | 162 |
| 5th | C4m#19-5th4w-NN6-83 | L | S | V | H | F | C | P | S | 163 |
| 4th | C4m3127-4th-NNS6-60 | S | S | W | H | F | C | G | L | 164 |
| 5th | C4m#19-5th2w-NNS6-53 | R | S | F | H | F | C | G | A | 165 |
| 5th | C4m#19-5th2w-NNS6-45 | W | S | V | H | F | C | W | R | 166 |
| 4th | C4m3127-4th-NNS6-3 | W | S | V | H | F | C | G | Q | 167 |
| 5th | C4m#19-5th2w-NNS6-6 | L | S | L | H | F | C | Q | R | 168 |
| 5th | C4m#19-5th2w-NNS6-27 | R | S | C | H | F | C | A | R | 169 |
| 5th | C4m#19-5th2w-NNS6-37 | W | S | I | H | F | C | S | R | 170 |
| 5th | C4m#19-5th2w-NNS6-33 | W | S | F | H | F | C | W | G | 171 |
| 5th | C4m#19-5th2w-NNS6-79 | V | S | R | H | F | C | V | A | 172 |
| 4th | C4m3127-4th-NNS6-85 | F | S | S | H | F | C | P | L | 173 |
| 5th | C4m#19-5th2w-NNS6-30 | L | S | W | H | F | C | G | R | 174 |
| 5th | C4m#19-5th4w-NN6-89 | I | S | Q | H | F | C | A | F | 175 |
| 5th | C4m#19-5th2w-NNS6-86 | W | S | L | H | F | C | R | S | 176 |
| 4th | C4m3127-4th-NNS6-30 | L | S | P | H | F | C | P | R | 177 |
| 4th | C4m3127-4th-NNS6-26 | W | S | C | H | F | C | P | F | 178 |
| 5th | C4m#19-5th2w-NNS6-74 | R | S | W | H | F | C | S | R | 179 |
| 4th | C4m3127-4th-NNS6-89 | F | S | A | H | F | C | M | R | 180 |
| 5th | C4m#19-5th2w-NNS6-84 | V | S | F | H | F | C | P | F | 181 |
| 5th | C4m#19-5th2w-NNS6-28 | R | S | V | H | F | C | P | C | 182 |
| 5th | C4m#19-5th2w-NNS6-4 | S | S | L | H | F | C | P | F | 183 |
| 5th | C4m#19-5th2w-NNS6-46 | F | S | Y | H | F | C | G | L | 184 |
| 5th | C4m#19-5th4w-NN6-61 | F | S | V | H | F | C | P | Y | 185 |
| 4th | C4m3127-4th-NNS6-94 | W | S | S | H | F | C | P | W | 186 |
| 5th | C4m#19-5th2w-NNS6-26 | V | S | I | H | F | C | S | A | 187 |
| 5th | C4m#19-5th4w-NN6-36 | W | S | A | H | F | C | C | R | 188 |
| 5th | C4m#19-5th4w-NN6-75 | K | S | V | H | F | C | C | A | 189 |
| 5th | C4m#19-5th2w-NNS6-70 | V | S | W | H | F | C | A | A | 190 |
| 5th | C4m#19-5th2w-NNS6-34 | Y | S | V | H | F | C | A | S | 191 |
| 5th | C4m#19-5th4w-NN6-94 | Y | S | L | H | F | C | P | C | 192 |
| 4th | C4m3127-4th-NNS6-66 | R | S | V | H | F | C | S | F | 193 |
| 5th | C4m#19-5th2w-NNS6-43 | W | S | S | H | F | C | A | A | 194 |
| 4th | C4m3127-4th-NNS6-7 | R | S | P | H | F | C | G | S | 195 |
| 5th | C4m#19-5th4w-NN6-72 | V | S | V | H | F | C | G | C | 196 |
| 4th | C4m3127-4th-NNS6-70 | H | S | R | H | F | C | L | S | 197 |
| 5th | C4m#19-5th2w-NNS6-68 | V | S | C | H | F | C | P | R | 198 |
| 5th | C4m#19-5th2w-NNS6-87 | F | S | V | H | F | C | A | L | 199 |
| 5th | C4m#19-5th2w-NNS6-66 | F | S | V | H | F | C | P | F | 200 |
| 4th | C4m3127-4th-NNS6-20 | L | S | L | H | F | C | P | F | 201 |
| 5th | C4m#19-5th2w-NNS6-61 | R | S | V | H | F | C | P | F | 202 |
| 4th | C4m3127-4th-NNS6-19 | W | S | W | H | F | C | P | H | 203 |
| 5th | C4m#19-5th4w-NN6-40 | R | S | H | H | F | C | M | L | 204 |
| 5th | C4m#19-5th4w-NN6-49 | R | S | F | H | F | C | S | C | 205 |
| 5th | C4m#19-5th4w-NN6-80 | F | S | W | H | F | C | A | V | 206 |
| 5th | C4m#19-5th2w-NNS6-80 | V | S | W | H | F | C | P | C | 207 |
| 4th | C4m3127-4th-NNS6-37 | R | S | I | H | F | C | G | C | 208 |
| 5th | C4m#19-5th2w-NNS6-93 | Q | S | R | H | F | C | C | R | 209 |
| 4th | C4m3127-4th-NNS6-95 | R | S | G | H | F | C | S | R | 210 |
| 5th | C4m#19-5th2w-NNS6-41 | R | S | D | H | F | C | C | R | 211 |

TABLE 11

| | | | | | | | | | | | | | | | | | Usage | |
|---|---|---|---|---|---|---|---|---|---|---|---|---|---|---|---|---|---|---|
| | | | | | | | | | | | | | | | | | frequency/ | |
| | Apparance frequency of amino acids | | | | | | | | | | | | | | | Usage | codon | Codon |
| #19 | M | H | P | F | L | P | I | V | S | H | H | F | C | E | R | frequency | numbers | numbers |
| W | 34 | | | 18 | | | | 21 | | 22 | | | | 3 | 5 | 103 | 103.00 | 1 |
| F | 14 | | | 68 | | | | 17 | | 9 | | | | 0 | 22 | 130 | 65.00 | 2 |
| Y | 0 | | | 27 | | | | 6 | | 3 | | | | 0 | 3 | 39 | 19.50 | 2 |
| V | 4 | | | 13 | | | | 24 | | 33 | | | | 2 | 7 | 83 | 20.75 | 4 |
| L | 21 | | | 1 | | | | 12 | | 21 | | | | 3 | 14 | 72 | 12.00 | 6 |
| I | 1 | | | 0 | | | | 2 | | 7 | | | | 0 | 3 | 13 | 4.33 | 3 |
| M | 10 | | | 1 | | | | 1 | | 1 | | | | 2 | 1 | 16 | 16.00 | 1 |
| P | 4 | | | 0 | | | | 0 | | 9 | | | | 57 | 0 | 70 | 17.50 | 4 |
| A | 5 | | | 3 | | | | 3 | | 3 | | | | 16 | 9 | 39 | 9.75 | 4 |
| G | 2 | | | 1 | | | | 0 | | 1 | | | | 22 | 4 | 30 | 7.50 | 4 |
| S | 6 | | | 3 | | | | 9 | | 8 | | | | 18 | 9 | 53 | 8.83 | 6 |
| T | 0 | | | 0 | | | | 0 | | 2 | | | | 0 | 0 | 2 | 0.50 | 4 |
| N | 0 | | | 0 | | | | 0 | | 0 | | | | 0 | 0 | 0 | 0.00 | 2 |
| Q | 2 | | | 1 | | | | 4 | | 1 | | | | 4 | 1 | 13 | 6.50 | 2 |
| D | 0 | | | 0 | | | | 0 | | 3 | | | | 0 | 0 | 3 | 1.50 | 2 |
| E | 0 | | | 0 | | | | 0 | | 0 | | | | 1 | 0 | 1 | 0.50 | 2 |
| K | 4 | | | 0 | | | | 4 | | 1 | | | | 0 | 3 | 12 | 6.00 | 2 |
| R | 31 | | | 0 | | | | 37 | | 9 | | | | 5 | 47 | 129 | 21.50 | 6 |
| H | 0 | | | 4 | | | | 1 | | 4 | | | | 0 | 2 | 11 | 5.50 | 2 |
| C | 3 | | | 1 | | | | 0 | | 4 | | | | 8 | 11 | 27 | 13.50 | 2 |

As a result, all amino acids except asparagine were used at positions 1, 4, 8, 10, +1 and +2 (corresponding to X₁, X₄, X₈, X₁₀, X₊₁ and X₊₂), and mutations at these positions were expected to have little effect on the presence or absence of binding activity. It was also observed that hydrophobic amino acids (especially aromatic amino acids) and amino acids with long side chains tend to appear more frequently, while hydrophilic amino acids with short side chains tend to appear less frequently. This was expected to be caused by the fact that hydrophobic bonds have been added due to the mutation to hydrophobic amino acids with long side chains, and the molecular structure of the peptide has been stabilized more tightly.

### INDUSTRIAL APPLICABILITY

According to the present invention, a cyclic peptide which inhibits CTLA-4 function is provided. The cyclic peptide of the present invention is useful as an immune checkpoint inhibitor and expected to be applied for a prophylactic or therapeutic drug against diseases (e.g., cancer) which can be treated or prevented by inhibiting CTLA-4 function.

This application is based on a patent application No. 2019-185266 filed in Japan (filing date: Oct. 8, 2019), the contents of which are incorporated in full herein.

1. A cyclic peptide which comprises the amino acid sequence represented by formula (I):
X₁-His-Pro-X₄-Leu-X₆-X₇-X₈-Ser-X₁₀-His-Phe (I)
in the cycle and has an activity to specifically bind to human CTLA-4, wherein X₁, X₄, X₆, X₇, X₈ and X₁₀ are each independently any amino acid.

2. The cyclic peptide according to claim 1, wherein the cyclic peptide comprises the amino acid sequence represented by formula (II):
X₀-X₁-His-Pro-X₄-Leu-X₆-X₇-X₈-Ser-X₁₀-His-Phe-X₀₀ (II)
wherein X₁, X₄, X₆, X₇, X₈ and X₁₀ are synonymous with those defined in formula (I), and X₀ and X₀₀ are each independently any amino acid, and wherein the peptide is cyclized by an intramolecular bond between X₀ and X₀₀.

3. The cyclic peptide according to claim 2, wherein the cyclic peptide comprises the amino acid sequence represented by formula (III):
(Y)m-X₀-X₁-His-Pro-X₄-Leu-X₆-X₇-X₈-Ser-X₁₀-His-Phe-X₀₀-(Z)n (III)
wherein
X₀, X₁, X₄, X₆, X₇, X₈, X₁₀ and X₀₀ are synonymous with those defined in formula
(Y)m is an amino acid sequence having a length of m amino acid(s),
(Z)n is an amino acid sequence having a length of n amino acid(s),
m is any integer selected from the group consisting of 0, 1 and 2, and
n is any integer selected from the group consisting of 0, 1 and 2.

4. The cyclic peptide according to claim 2, wherein X₀ and X₀₀ are each independently an amino acid having a side chain containing a thiol group.

5. The cyclic peptide according to claim 4, wherein
X₀ is Cys, and
X₀₀ is Cys.

6. The cyclic peptide according to claim 4, wherein the peptide is cyclized by an intramolecular disulfide bond between a thiol group in the side chain of X₀ and a thiol group in the side chain of X₀₀.

7. The cyclic peptide according to claim 1, wherein
X₆ is Leu, Pro, Gln, Lys or Arg, and
X₇ is Val, Leu, Ile or Thr.

8. The cyclic peptide according to claim 7, wherein
X₆ is Pro, and
X₇ is Ile.

9. The cyclic peptide according to claim 3, wherein
n is 2,
(Z)n consists of an amino acid sequence represented by X₊₁-X₊₂, and
X₊₁ and X₊₂ are each independently any amino acid.

10. The cyclic peptide according to claim 3, wherein
m is 2,
(Y)m consists of an amino acid sequence represented by X₋₂-X₋₁, and
X₋₂ and X₋₁ are each independently any amino acid.

11. The cyclic peptide according to claim 10, wherein
X₋₂ is Gly, and
X₋₁ is Gly, Ser or Asp.

12. The cyclic peptide according to claim 1, wherein the amino acid sequence represented by formula (I) consists of
(1a) an amino acid sequence from the position 4 to position 15 in the amino acid sequence represented by any of SEQ ID NOs: 30-211, or
(2a) an amino acid sequence having a substitution at least at one amino acid corresponding to one selected from X₁, X₄, X₆, X₇, X₈ and X₁₀ in an amino acid sequence from the position 4 to position 15 in the amino acid sequence represented by any of SEQ ID NOs: 30-211.

13. The cyclic peptide according to claim 2, wherein the amino acid sequence represented by formula (II) consists of
(1b) an amino acid sequence from position 3 to position 16 in the amino acid sequence represented by any of SEQ ID NOs: 30-211, or
(2b) an amino acid sequence having a substitution at least at one amino acid corresponding to one selected from X₁, X₄, X₆, X₇, X₈ and X₁₀ in an amino acid sequence from position 3 to position 16 in the amino acid sequence represented by any of SEQ ID NOs: 30-211.

14. The cyclic peptide according to claim 3, wherein the amino acid sequence represented by formula (III) consists of
(1c) the amino acid sequence represented by any of SEQ ID NOs: 30-211, or
(2c) an amino acid sequence having a substitution at least at one amino acid corresponding to one selected from X₋₂, X₋₁, X₁, X₄, X₆, X₇, X₈, X₁₀, X₊₁ and X₊₂ in the amino acid sequence represented by any of SEQ ID NOs: 30-211.

15. The cyclic peptide according to claim 1, wherein the cyclic peptide has an activity to inhibit the interaction between human CTLA-4 and human CD80.

16. A pharmaceutical composition which comprises the cyclic peptide according to claim 1, and a pharmaceutically acceptable carrier or excipient.

17.-20. (canceled)

21. A method for preventing or treating a tumor in a subject, which comprises administering an effective amount of the cyclic peptide according to claim 1 to the subject.

22.-23. (canceled)
